SEQUENCE LISTING (165 sequences)

SEQ ID NO: 1 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 2 - protein, 357 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVNEELPSINTYFPQNILESHFNRISLLEK

SEQ ID NO: 3 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 5 - protein, 227 aa, Homo sapiens
MKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 6 - protein, 660 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCDR

SEQ ID NO: 7 - protein, 862 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRHVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 8 - protein, 52 aa, Artificial Sequence
  Synthetic
EPPTQKPKKIVNAKKDVVNTKMFEELKSRLDTLAQEVALLKEQQALQTVCLK

SEQ ID NO: 9 - protein, 54 aa, Artificial Sequence
  Synthetic
EPPTQKPKKIVNAKKDVVNTKMFEELKARLDTLSQEVALLKEQQALQTVSLKGS

SEQ ID NO: 10 - protein, 49 aa, Artificial Sequence
  Synthetic
EPPTQKPKKIVNAKKDVVNTKMFEELKSRLDTLAQEVALLKEQQALQTV

SEQ ID NO: 11 - protein, 47 aa, Artificial Sequence
  Synthetic
KPKKIVNAKKDVVNTKMFEELKSRLDTLSQEVALLKEQQALQTVSLK

SEQ ID NO: 12 - protein, 43 aa, Artificial Sequence
  Synthetic
IVNAKKDVVNTKMFEELKSRLDTLSQEVALLKEQQALQTVSLK

SEQ ID NO: 13 - protein, 37 aa, Artificial Sequence
  Synthetic
DVVNTKMFEELKSRLDTLSQEVALLKEQQALQTVSLK

SEQ ID NO: 14 - protein, 33 aa, Artificial Sequence
  Synthetic
TKMFEELKSRLDTLSQEVALLKEQQALQTVSLK

SEQ ID NO: 15 - protein, 29 aa, Artificial Sequence
  Synthetic
EELKSRLDTLSQEVALLKEQQALQTVSLK

SEQ ID NO: 16 - protein, 25 aa, Artificial Sequence
  Synthetic
SRLDTLSQEVALLKEQQALQTVSLK

SEQ ID NO: 17 - protein, 43 aa, Artificial Sequence
  Synthetic
KPKKIVNAKKDVVNTKMFEELKSRLDTLSQEVALLKEQQALQT

SEQ ID NO: 18 - protein, 41 aa, Artificial Sequence
  Synthetic
KPKKIVNAKKDVVNTKMFEELKSRLDTLSQEVALLKEQQAL

SEQ ID NO: 19 - protein, 38 aa, Artificial Sequence
  Synthetic
KPKKIVNAKKDVVNTKMFEELKSRLDTLSQEVALLKEQ

SEQ ID NO: 20 - protein, 34 aa, Artificial Sequence
  Synthetic
KPKKIVNAKKDVVNTKMFEELKSRLDTLSQEVAL

SEQ ID NO: 21 - protein, 31 aa, Artificial Sequence
  Synthetic
KPKKIVNAKKDVVNTKMFEELKSRLDTLSQE

SEQ ID NO: 22 - protein, 40 aa, Artificial Sequence
  Synthetic
IVNAKKDVVNTKMFEELKSRLDTLAQEVALLKEQQALQTV

SEQ ID NO: 23 - protein, 33 aa, Artificial Sequence
  Synthetic
VVNTKMFEELKSRLDTLAQEVALLKEQQALQTV

SEQ ID NO: 24 - protein, 53 aa, Artificial Sequence
  Synthetic
EPPTQKPKKIVNAKKDVVNTKMFEELKSRLDTLAQEVALLKEQQALQTVSLKG

SEQ ID NO: 25 - protein, 52 aa, Artificial Sequence
  Synthetic
EPPTQKPKKIVNAKKDVVNTKMFEELKSRLDTLAQEVALLKEQQALQTVSLK

SEQ ID NO: 26 - protein, 51 aa, Artificial Sequence
  Synthetic
EPPTQKPKKIVNAKKDVVNTKMFEELKSRLDTLAQEVALLKEQQALQTVSL

SEQ ID NO: 27 - protein, 50 aa, Artificial Sequence
  Synthetic
EPPTQKPKKIVNAKKDVVNTKMFEELKSRLDTLAQEVALLKEQQALQTVS

SEQ ID NO: 28 - protein, 49 aa, Artificial Sequence; identical to SEQ ID NO: 10
  Synthetic

SEQ ID NO: 29 - protein, 48 aa, Artificial Sequence
  Synthetic
EPPTQKPKKIVNAKKDVVNTKMFEELKSRLDTLAQEVALLKEQQALQT

SEQ ID NO: 30 - protein, 52 aa, Artificial Sequence
  Synthetic
PPTQKPKKIVNAKKDVVNTKMFEELKSRLDTLAQEVALLKEQQALQTVSLKG

SEQ ID NO: 31 - protein, 48 aa, Artificial Sequence
  Synthetic
PPTQKPKKIVNAKKDVVNTKMFEELKSRLDTLAQEVALLKEQQALQTV

SEQ ID NO: 32 - protein, 51 aa, Artificial Sequence
  Synthetic
PTQKPKKIVNAKKDVVNTKMFEELKSRLDTLAQEVALLKEQQALQTVSLKG

SEQ ID NO: 33 - protein, 50 aa, Artificial Sequence
  Synthetic
TQKPKKIVNAKKDVVNTKMFEELKSRLDTLAQEVALLKEQQALQTVSLKG

SEQ ID NO: 34 - protein, 49 aa, Artificial Sequence
  Synthetic
QKPKKIVNAKKDVVNTKMFEELKSRLDTLAQEVALLKEQQALQTVSLKG

SEQ ID NO: 35 - protein, 48 aa, Artificial Sequence
  Synthetic
KPKKIVNAKKDVVNTKMFEELKSRLDTLAQEVALLKEQQALQTVSLKG

SEQ ID NO: 36 - protein, 47 aa, Artificial Sequence
  Synthetic
PKKIVNAKKDVVNTKMFEELKSRLDTLAQEVALLKEQQALQTVSLKG

SEQ ID NO: 37 - protein, 46 aa, Artificial Sequence
  Synthetic
KKIVNAKKDVVNTKMFEELKSRLDTLAQEVALLKEQQALQTVSLKG

SEQ ID NO: 38 - protein, 45 aa, Artificial Sequence
  Synthetic
KIVNAKKDVVNTKMFEELKSRLDTLAQEVALLKEQQALQTVSLKG

SEQ ID NO: 39 - protein, 44 aa, Artificial Sequence
  Synthetic
IVNAKKDVVNTKMFEELKSRLDTLAQEVALLKEQQALQTVSLKG

SEQ ID NO: 40 - protein, 43 aa, Artificial Sequence
  Synthetic
VNAKKDVVNTKMFEELKSRLDTLAQEVALLKEQQALQTVSLKG

SEQ ID NO: 41 - protein, 42 aa, Artificial Sequence
  Synthetic
NAKKDVVNTKMFEELKSRLDTLAQEVALLKEQQALQTVSLKG

SEQ ID NO: 42 - protein, 41 aa, Artificial Sequence
  Synthetic
AKKDVVNTKMFEELKSRLDTLAQEVALLKEQQALQTVSLKG

SEQ ID NO: 43 - protein, 40 aa, Artificial Sequence
  Synthetic
KKDVVNTKMFEELKSRLDTLAQEVALLKEQQALQTVSLKG

SEQ ID NO: 44 - protein, 39 aa, Artificial Sequence
  Synthetic
KDVVNTKMFEELKSRLDTLAQEVALLKEQQALQTVSLKG

SEQ ID NO: 45 - protein, 37 aa, Artificial Sequence
  Synthetic
VVNTKMFEELKSRLDTLAQEVALLKEQQALQTVSLKG

SEQ ID NO: 46 - protein, 36 aa, Artificial Sequence
  Synthetic
VNTKMFEELKSRLDTLAQEVALLKEQQALQTVSLKG

SEQ ID NO: 47 - protein, 35 aa, Artificial Sequence
  Synthetic
VNTKMFEELKSRLDTLAQEVALLKEQQALQTVSLK

SEQ ID NO: 48 - protein, 34 aa, Artificial Sequence
  Synthetic
NTKMFEELKSRLDTLAQEVALLKEQQALQTVSLK

SEQ ID NO: 49 - protein, 33 aa, Artificial Sequence
  Synthetic
TKMFEELKSRLDTLAQEVALLKEQQALQTVSLK

SEQ ID NO: 50 - protein, 32 aa, Artificial Sequence
  Synthetic
KMFEELKSRLDTLAQEVALLKEQQALQTVSLK

SEQ ID NO: 51 - protein, 31 aa, Artificial Sequence
  Synthetic
MFEELKSRLDTLAQEVALLKEQQALQTVSLK

SEQ ID NO: 52 - protein, 33 aa, Artificial Sequence; identical to SEQ ID NO: 23
  Synthetic

SEQ ID NO: 53 - protein, 32 aa, Artificial Sequence
  Synthetic
VVNTKMFEELKSRLDTLAQEVALLKEQQALQT

SEQ ID NO: 54 - protein, 30 aa, Artificial Sequence
  Synthetic
VNTKMFEELKSRLDTLAQEVALLKEQQALQ

SEQ ID NO: 55 - protein, 35 aa, Artificial Sequence
  Synthetic
NTKMFEELKSRLDTLAQEVALLKEQQALQTVSLKG

SEQ ID NO: 56 - protein, 34 aa, Artificial Sequence
  Synthetic
TKMFEELKSRLDTLAQEVALLKEQQALQTVSLKG

SEQ ID NO: 57 - protein, 33 aa, Artificial Sequence
  Synthetic
KMFEELKSRLDTLAQEVALLKEQQALQTVSLKG

SEQ ID NO: 58 - protein, 32 aa, Artificial Sequence
  Synthetic
MFEELKSRLDTLAQEVALLKEQQALQTVSLKG

SEQ ID NO: 59 - protein, 52 aa, Artificial Sequence
  Synthetic
EGPTQKPKKIVNAKKDVVNTKMFEELKSRLDTLAQEVALLKEQQALQTVSLK

SEQ ID NO: 60 - protein, 49 aa, Artificial Sequence
  Synthetic
EGPTQKPKKIVNAKKDVVNTKMFEELKSRLDTLAQEVALLKEQQALQTV

SEQ ID NO: 61 - protein, 48 aa, Artificial Sequence
  Synthetic
EGPTQKPKKIVNAKKDVVNTKMFEELKSRLDTLAQEVALLKEQQALQT

SEQ ID NO: 62 - protein, 47 aa, Artificial Sequence
  Synthetic
EGPTQKPKKIVNAKKDVVNTKMFEELKSRLDTLAQEVALLKEQQALQ

SEQ ID NO: 63 - protein, 43 aa, Artificial Sequence
  Synthetic
IVNAKKDVVNTKMFEELKSRLDTLAQEVALLKEQQALQTVSLK

SEQ ID NO: 64 - protein, 40 aa, Artificial Sequence; identical to SEQ ID NO: 22
  Synthetic

SEQ ID NO: 65 - protein, 39 aa, Artificial Sequence
  Synthetic
IVNAKKDVVNTKMFEELKSRLDTLAQEVALLKEQQALQT

SEQ ID NO: 66 - protein, 38 aa, Artificial Sequence
  Synthetic
IVNAKKDVVNTKMFEELKSRLDTLAQEVALLKEQQALQ

SEQ ID NO: 67 - protein, 35 aa, Artificial Sequence; identical to SEQ ID NO: 47
  Synthetic

SEQ ID NO: 68 - protein, 32 aa, Artificial Sequence
  Synthetic
VNTKMFEELKSRLDTLAQEVALLKEQQALQTV

SEQ ID NO: 69 - protein, 31 aa, Artificial Sequence
  Synthetic
VNTKMFEELKSRLDTLAQEVALLKEQQALQT

SEQ ID NO: 70 - protein, 30 aa, Artificial Sequence; identical to SEQ ID NO: 54
  Synthetic

SEQ ID NO: 71 - protein, 40 aa, Artificial Sequence
  Synthetic
MIVNAKKDVVNTKMFEELKSRLDTLAQEVALLKEQQALQT

SEQ ID NO: 72 - protein, 32 aa, Artificial Sequence
  Synthetic
MVNTKMFEELKSRLDTLAQEVALLKEQQALQT

SEQ ID NO: 73 - protein, 53 aa, Artificial Sequence
  Synthetic
EPPTQKPKKIVNAKKDVVNTKMFEELKARLDTLSQEVALLKEQQALQTVSLKG

SEQ ID NO: 74 - protein, 52 aa, Artificial Sequence
  Synthetic
EPPTQKPKKIVNAKKDVVNTKMFEELKARLDTLSQEVALLKEQQALQTVSLK

SEQ ID NO: 75 - protein, 51 aa, Artificial Sequence
  Synthetic
EPPTQKPKKIVNAKKDVVNTKMFEELKARLDTLSQEVALLKEQQALQTVSL

SEQ ID NO: 76 - protein, 50 aa, Artificial Sequence
  Synthetic
EPPTQKPKKIVNAKKDVVNTKMFEELKARLDTLSQEVALLKEQQALQTVS

SEQ ID NO: 77 - protein, 49 aa, Artificial Sequence
  Synthetic
EPPTQKPKKIVNAKKDVVNTKMFEELKARLDTLSQEVALLKEQQALQTV

SEQ ID NO: 78 - protein, 52 aa, Artificial Sequence
  Synthetic
PPTQKPKKIVNAKKDVVNTKMFEELKARLDTLSQEVALLKEQQALQTVSLKG

SEQ ID NO: 79 - protein, 51 aa, Artificial Sequence
  Synthetic
PTQKPKKIVNAKKDVVNTKMFEELKARLDTLSQEVALLKEQQALQTVSLKG

SEQ ID NO: 80 - protein, 50 aa, Artificial Sequence
  Synthetic
TQKPKKIVNAKKDVVNTKMFEELKARLDTLSQEVALLKEQQALQTVSLKG

SEQ ID NO: 81 - protein, 49 aa, Artificial Sequence
  Synthetic
QKPKKIVNAKKDVVNTKMFEELKARLDTLSQEVALLKEQQALQTVSLKG

SEQ ID NO: 82 - protein, 48 aa, Artificial Sequence
  Synthetic
KPKKIVNAKKDVVNTKMFEELKARLDTLSQEVALLKEQQALQTVSLKG

SEQ ID NO: 83 - protein, 47 aa, Artificial Sequence
  Synthetic
PKKIVNAKKDVVNTKMFEELKARLDTLSQEVALLKEQQALQTVSLKG

SEQ ID NO: 84 - protein, 46 aa, Artificial Sequence
  Synthetic
KKIVNAKKDVVNTKMFEELKARLDTLSQEVALLKEQQALQTVSLKG

SEQ ID NO: 85 - protein, 45 aa, Artificial Sequence
  Synthetic
KIVNAKKDVVNTKMFEELKARLDTLSQEVALLKEQQALQTVSLKG

SEQ ID NO: 86 - protein, 44 aa, Artificial Sequence
  Synthetic
IVNAKKDVVNTKMFEELKARLDTLSQEVALLKEQQALQTVSLKG

SEQ ID NO: 87 - protein, 43 aa, Artificial Sequence
  Synthetic
VNAKKDVVNTKMFEELKARLDTLSQEVALLKEQQALQTVSLKG

SEQ ID NO: 88 - protein, 42 aa, Artificial Sequence
  Synthetic
NAKKDVVNTKMFEELKARLDTLSQEVALLKEQQALQTVSLKG

SEQ ID NO: 89 - protein, 41 aa, Artificial Sequence
  Synthetic
AKKDVVNTKMFEELKARLDTLSQEVALLKEQQALQTVSLKG

SEQ ID NO: 90 - protein, 40 aa, Artificial Sequence
  Synthetic
KKDVVNTKMFEELKARLDTLSQEVALLKEQQALQTVSLKG

SEQ ID NO: 91 - protein, 39 aa, Artificial Sequence
  Synthetic
KDVVNTKMFEELKARLDTLSQEVALLKEQQALQTVSLKG

SEQ ID NO: 92 - protein, 37 aa, Artificial Sequence
  Synthetic
VVNTKMFEELKARLDTLSQEVALLKEQQALQTVSLKG

SEQ ID NO: 93 - protein, 36 aa, Artificial Sequence
  Synthetic
VNTKMFEELKARLDTLSQEVALLKEQQALQTVSLKG

SEQ ID NO: 94 - protein, 35 aa, Artificial Sequence
  Synthetic
VNTKMFEELKARLDTLSQEVALLKEQQALQTVSLK

SEQ ID NO: 95 - protein, 34 aa, Artificial Sequence
  Synthetic
NTKMFEELKARLDTLSQEVALLKEQQALQTVSLK

SEQ ID NO: 96 - protein, 33 aa, Artificial Sequence
  Synthetic
TKMFEELKARLDTLSQEVALLKEQQALQTVSLK

SEQ ID NO: 97 - protein, 31 aa, Artificial Sequence
  Synthetic
MFEELKARLDTLSQEVALLKEQQALQTVSLK

SEQ ID NO: 98 - protein, 71 aa, Artificial Sequence
  Synthetic
MGSHHHHHGSIQGRSPGTEPPTQKPKKIVNAKKDVVNTKMFEELKSRLDTLAQEVALLKEQQALQTVSLKG

SEQ ID NO: 99 - protein, 52 aa, Artificial Sequence; identical to SEQ ID NO: 8
  Synthetic

SEQ ID NO: 100 - protein, 52 aa, Artificial Sequence
  Synthetic
ESPTPKAKKAANAKKDLVSSKMFEELKNRMDVLAQEVALLKEKQALQTVCLK

SEQ ID NO: 101 - protein, 52 aa, Artificial Sequence
  Synthetic
QQNGKGRQKPAASKKDGVSLKMIEDLKAMIDNISQEVALLKEKQALQTVCLK

SEQ ID NO: 102 - protein, 52 aa, Artificial Sequence
  Synthetic
ETPTPKAKKAANAKKDAVSPKMLEELKTQLDSLAQEVALLKEQQALQTVCLK

SEQ ID NO: 103 - protein, 49 aa, Artificial Sequence
  Synthetic
QQTSSKKKGGKKDAENNAAIEELKKQIDNIVLELNLLKEQQALQSVCLK

SEQ ID NO: 104 - protein, 49 aa, Artificial Sequence
  Synthetic
QQNGKKNKQNNKDVVSMKMYEDLKKKVQNIEEDVIHLKEQQALQTICLK

SEQ ID NO: 105 - protein, 48 aa, Artificial Sequence
  Synthetic
EQSLTKRKNGKKESNSAAIEELKKQIDQIIQDLNLLKEQQALQTVCLK

SEQ ID NO: 106 - protein, 52 aa, Artificial Sequence
  Synthetic
QTSCHASKFKARKHSKRRVKEKDGDLKTQVEKLWREVNALKEMQALQTVCLR

SEQ ID NO: 107 - protein, 38 aa, Artificial Sequence
  Synthetic
KPSKSGKGKDDLRNEIDKLWREVNSLKEMQALQTVCLK

SEQ ID NO: 108 - protein, 52 aa, Artificial Sequence
  Synthetic
XXXXXXXXXXXXXXXXXXXXXXXXXLXXXXXXLXXEVXXLKEXQALQTVCLX

SEQ ID NO: 109 - protein, 137 aa, Artificial Sequence
  Synthetic
ALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 110 - protein, 126 aa, Artificial Sequence
  Synthetic
NKLHAGSMGKKSGKKFFVTNHERMPFSKVKALCSELRGTVAIPRNAEENKAIQEVAKTSAFLGITDEVTEGQFMYVTGGRLTYSNWKKDEPNDHGSGEDCVTIVDNGLWNDISCQASHTAVCSFPA

SEQ ID NO: 111 - protein, 127 aa, Artificial Sequence
  Synthetic
KKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCAF

SEQ ID NO: 112 - protein, 123 aa, Artificial Sequence
  Synthetic
KVYWFCYGMKCYYFVMDRKTWSGCKQTCQSSSLSLLKIDDEDELKFLQLLVVPSDSCWVGLSYDNKKDWAWIDNRPSKLALNTRKYNIRDRGGCMLLSKTRLDNGNCDQVFICICGKRLDKFP

SEQ ID NO: 113 - protein, 128 aa, Artificial Sequence
  Synthetic
CPVNWVEHERSCYWFSRSGKAWADADNYCRLEDAHLVVVTSWEEQLFVQHHIGPVNTWMGLHDQNGPWKWVDGTDYETGFKNWRPEQPDDWYGHGLGGGEDCAHFTDDGRWNDDVCQRPYRWVCSTEL

SEQ ID NO: 114 - protein, 147 aa, Artificial Sequence
  Synthetic
GIPKCPEDWGASSRTSLCFKLYAKGKHEKKTWFESRDFCRALGGDLASINNKEEQQTIWRLITASGSYHKLFWLGLTYGSPSEGFTWSDGSPVSYENWAYGEPNNYQNVEYCGELKGDPTMSWNDINCEHLNNWICQIQKGQTPKPD

SEQ ID NO: 115 - protein, 129 aa, Artificial Sequence
  Synthetic
DCLSGWSSYEGHCYKAFSKYKTWEDAERVCTEQAKGAHLVSIESSGEADFVAQLVTQNMKRLDFYIWIGLRVQGKVKQCNSEWSDGSSVSYENWIEAESKTCLGLEKETDFRKWVNIYCGQQNPFVCEA

SEQ ID NO: 116 - protein, 122 aa, Artificial Sequence
  Synthetic
DCPSDWSSYEGHCYKPFSEPKNWADAENFCTQQHAGGHLVSFQSSEEADFVVKLAFQTFHSIFWMGLSNVWNQCNWQWSNAAMLRYKAWAEESYCVYFKSTNNKWRSRACRMMAQFVCEFQA

SEQ ID NO: 117 - protein, 135 aa, Artificial Sequence
  Synthetic
ARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFGKWKDVPCEDKFSFVCKFKN

SEQ ID NO: 118 - protein, 123 aa, Artificial Sequence
  Synthetic
DYEILFSDETMNYADAGTYCGSRGMALVSSAMRDSTMVKAILAFTEVKGHDYWVGADNLQDGAYNFNWNDGVSLPTDSDLWSPNEPSNPQSWQLCVQIWSKYNLLDDVGCGGARRVICEKELD

SEQ ID NO: 119 - protein, 202 aa, Homo sapiens
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 120 - protein, 202 aa, Mus musculus
MGFWGTYLLFCLFSFLSQLTAESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 121 - protein, 201 aa, Gallus gallus
MALRGACLLLCLVSLAHISVQQNGKGRQKPAASKKDGVSLKMIEDLKAMIDNISQEVALLKEKQALQTVCLKGTKIHLKCFLAFSESKTYHEASEHCISQGGTLGTPQGGEENDALYDYMRKSIGNEAEIWLGLNDMVAEGKWVDMTGSPIRYKNWETEITTQPDGGKLENCAALSGVAVGKWFDKRCKEQLPYVCQFMIV

SEQ ID NO: 122 - protein, 202 aa, Bos taurus
MELWGPCVLLCLFSLLTQVTAETPTPKAKKAANAKKDAVSPKMLEELKTQLDSLAQEVALLKEQQALQTVCLKGTKVHMKCFLAFVQAKTFHEASEDCISRGGTLGTPQTGSENDALYEYLRQSVGSEAEVWLGFNDMASEGSWVDMTGGHIAYKNWETEITAQPDGGKVENCATLSGAANGKWFDKRCRDKLPYVCQFAIV

SEQ ID NO: 123 - protein, 198 aa, Salmo salar
MRVSGVRLLFCLLLLGQSTFQQTSSKKKGGKKDAENNAAIEELKKQIDNIVLELNLLKEQQALQSVCLKGIKIIGKCFLADTAKKIYHTAYDDCIAKGGTISTPLTGDENDQLVDYVRRSIGPEEHIWLGINDMVTEGEWLDQAGTNLRFKNWETDITNQPDGGRTHNCAILSTTANGKWFDESCRVEKASVCEFNIV

SEQ ID NO: 124 - protein, 198 aa, Silurana tropicalis
MEYRRACILLCLFCFVQVTLQQNGKKNKQNNKDVVSMKMYEDLKKKVQNIEEDVIHLKEQQALQTICLKGMKIYNKCFLAFNELKTYHQASDVCFAQGGTLSTPETGDENDSLYDYVRKSIGSSAEIWIGINDMATEGTWLDLTGSPISFKHWETEITTQPDGGKQENCAALSASAIGRWFDKNCKTELPFVCQFSIV

SEQ ID NO: 125 - protein, 223 aa, Danio rerio
MRDDSDKVPSLLTDYILKGCTYAEEKMDLKAVKFLLCVICLVKSSPEQSLTKRKNGKKESNSAAIEELKKQIDQIIQDLNLLKEQQALQTVCLKGFKIPGKCFLVDTVKKDFHSANDDCIAKGGILSTPMSGHENDQLQEYVQQTVGPETHIWLGVNDMIKEGEWIDLTGSPIRFKNWESEITHQPDGGRTHNCAVLSSTANGKWFDEDCRGEKASVCQFNIV

SEQ ID NO: 126 - protein, 197 aa, Bos taurus
MAKNGLVIYILVITLLLDQTSCHASKFKARKHSKRRVKEKDGDLKTQVEKLWREVNALKEMQALQTVCLRGTKFHKKCYLAAEGLKHFHEANEDCISKGGTLVVPRSADEINALRDYGKRSLPGVNDFWLGINDMVAEGKFVDINGLAISFLNWDQAQPNGGKRENCALFSQSAQGKWSDEACHSSKRYICEFTIPQ

SEQ ID NO: 127 - protein, 166 aa, Carcharhinus springeri
SKPSKSGKGKDDLRNEIDKLWREVNSLKEMQALQTVCLKGTKIHKKCYLASRGSKSYHAANEDCIAQGGTLSIPRSSDEGNSLRSYAKKSLVGARDFWIGVNDMTTEGKFVDVNGLPITYFNWDRSKPVGGTRENCVAASTSGQGKWSDDVCRSEKRYICEYLIPV

SEQ ID NO: 128 - protein, 204 aa, Artificial Sequence
  Synthetic
MELWGAXXLLCLFSXLXQVTAXXXXXKAKKXXXXXKKDXVSXKMXEELKXQIDXLAQEVXLLKEQQALQTVCLKGTKIHXKCFLAFTQXKTFHEASEDCISQGGTLSTPQXGDENDALXXYXRXSVGNEAXIWLGXNDMAAEGXWVDMTGSXIXYKNWETEITXQPDGGKXENCAALSXXANGKWFDKXCRDELPYVCQFXIVX

SEQ ID NO: 129 - DNA, 240 bp, Artificial Sequence
  Synthetic
gaggccgagatctggctgggcctgaacgacatgnnknnknnknnknnknnknnktgggtggatatgactggcgcccgcatcgcctacaagaactgggaaactgagatcaccgcccaacctgatggcggcgcaaccgagaactgcgcggtcctgtctggcgccgccaacggcaagtggttcgacaagcgctgcagggatcaattgccctacatctgccagttcgggatcgtggcggccgca

SEQ ID NO: 130 - protein, 80 aa, Artificial Sequence
  Synthetic
EAEIWLGLNDMXXXXXXXWVDMTGARIAYKNWETEITAQPDGGATENCAVLSGAANGKWFDKRCRDQLPYICQFGIVAAA

SEQ ID NO: 131 - protein, 137 aa, Artificial Sequence; identical to SEQ ID NO: 109
  Synthetic

SEQ ID NO: 132 - DNA, 414 bp, Artificial Sequence
  Synthetic
caggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgactggcgcgcgtatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtg

SEQ ID NO: 133 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSALTNTWVDMTGARIAYKNWEPPGPHHPMGGFGVFGENCAVLSGAANGKWFDKRCRDQLPYICQFGIV

SEQ ID NO: 134 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSALTNTWVDMTGARIAYKNWEPPPPHHPMGGFGVFGENCAVLSGAANGKWFDKRCRDQLPYICQFGIV

SEQ ID NO: 135 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSALTNTWVDMTGARIAYKNWERPALVQPRGGFGVFGENCAVLSGAANGKWFDKRCRDQLPYICQFGIV

SEQ ID NO: 136 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSALTNTWVDMTGARIAYKNWERPPLYQPGGGWGVFGENCAVLSGAANGKWFDKRCRDQLPYICQFGIV

SEQ ID NO: 137 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSALTNTWVDMTGARIAYKNWERTPPWQPEGGFGYFGENCAVLSGAANGKWFDKRCRDQLPYICQFGIV

SEQ ID NO: 138 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSLRTDTWVDMTGARIAYKNWETEITAQPDGGFGVFGENCAVLSGAANGKWFDKRCRDQLPYICQFGIV

SEQ ID NO: 139 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSLRTNTWVDMTGARIAYKNWETEITAQPDGGFGVFGENCAVLSGAANGKWFDKRCRDQLPYICQFGIV

SEQ ID NO: 140 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSALTNTWVDMTGARIAYKNWERPPLYQPGGGFGVFGENCAVLSGAANGKWFDKRCRDQLPYICQFGIV

SEQ ID NO: 141 - protein, 140 aa, Artificial Sequence
  Synthetic
ALQTVCLKGTKVHMKCFLAFTQTKTFHEASEDCISRGGTLSTPQTGSENDALYEYLRQSVGNEAEIWLGLNGSALTNTWVDMTGARIAYKNWATEITAQPDGGFGVFGENCAVLSGAANGKWFDKRCRDQLPYICQFGIV

SEQ ID NO: 142 - protein, 181 aa, Artificial Sequence
  Synthetic
EPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 143 - DNA, 546 bp, Artificial Sequence
  Synthetic
gagccaccaacccagaagcccaagaagattgtaaatgccaagaaagatgttgtgaacacaaagatgtttgaggagctcaagagccgtctggacaccctggcccaggaggtggccctgctgaaggagcagcaggccctgcagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggcgcccgcatcgcctacaagaactgggagactgagatcaccgcgcaacccgatggcggcaagaccgagaactgcgcggtcctgtcaggcgcggccaacggcaagtggttcgacaagcgctgccgcgatcagctgccctacatctgccagttcgggatcgtgtag

SEQ ID NO: 144 - DNA, 546 bp, Artificial Sequence
  Synthetic
gagtcacccactcccaaggccaagaaggctgcaaatgccaagaaagatttggtgagctcaaagatgttcgaggagctcaagaacaggatggatgtcctggcccaggaggtggccctgctgaaggagaagcaggccttacagactgtgtgcctgaagggcaccaaggtgaacttgaagtgcctcctggccttcacccaaccgaagaccttccatgaggcgagcgaggactgcatctcgcaagggggcacgctgggcaccccgcagtcagagctagagaacgaggcgctgttcgagtacgcgcgccacagcgtgggcaacgatgcgaacatctggctgggcctcaacgacatggccgcggaaggcgcctgggtggacatgaccggcggcctcctggcctacaagaactgggagacggagatcacgacgcaacccgacggcggcaaagccgagaactgcgccgccctgtctggcgcagccaacggcaagtggttcgacaagcgatgccgcgatcagttgccctacatctgccagtttgccattgtgtag

SEQ ID NO: 145 - protein, 181 aa, Artificial Sequence
  Synthetic
ESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 146 - skipped
  Synthetic

SEQ ID NO: 147 - skipped
  Synthetic

SEQ ID NO: 148 - skipped
  Synthetic

SEQ ID NO: 149 - skipped
  Synthetic

SEQ ID NO: 150 - DNA, 4779 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctaagtgacgatatcctgacctaaggtacctaagtgacgatatcctgacctaactgcagggatcaattgccctacatctgccagttcgggatcgtggcggccgcaggtgcgccggtgccgtatccggatccgctggaaccgcgtgccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 151 - DNA, 5747 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgggatctcgatcccgcgaaattaatacgactcactataggggaattgtgagcggataacaattcccctctagaaataattttgtttaactttaagaaggagatatacatatgaaataccttcttccgactgctgctgctggtcttttactgctggctgctcagccggctatggctgctggtggtggttctgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 152 - DNA, 10975 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 153 - DNA, 5774 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgggatctcgatcccgcgaaattaatacgactcactataggggaattgtgagcggataacaattcccctctagaaataattttgtttaactttaagaaggagatatacatatgaaataccttcttccgactgctgctgctggtcttttactgctggctgctcagccggctatggctgctggtggtggttctgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgcccaccaccaccaccaccactaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 154 - DNA, 4649 bp, Artificial Sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattcaccatgaaaaagacagctatcgcgattgcagtggcactggctggtttcgctaccgttgcgcaagcttctgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 155 - DNA, 10972 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacgtgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 156 - DNA, 10972 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccaggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 157 - DNA, 10969 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagtgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 158 - DNA, 10975 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtcagcctgaaggggaccaaggtgcacatgaaaagctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 159 - DNA, 10927 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 160 - DNA, 4641 bp, Artificial Sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattcaccatgaaaaagacagctatcgcgattgcagtggcactggctggtttcgctaccgttgcgcaagcttctgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccgcagttcgaaaaataactcgagataaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 161 - DNA, 11011 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccccagttcgagaagtgactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 162 - DNA, 5783 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgggatctcgatcccgcgaaattaatacgactcactataggggaattgtgagcggataacaattcccctctagaaataattttgtttaactttaagaaggagatatacatatgaaataccttcttccgactgctgctgctggtcttttactgctggctgctcagccggctatggctgctggtggtggttctgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccgcagttcgaaaaataactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 163 - DNA, 4792 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccttacagactgtgtgcctgaagggcaccaaggtgaacttgaagtgcctcctggccttcacccaaccgaagaccttccatgaggcgagcgaggactgcatctcgcaagggggcacgctgggtaccccgcagtcagagctggagaacgaggcgctgttcgaatacgcgcgccacagcgtgggcaacgatgcgaacatctggctgggcctcaacgacatggccgcggaaggcgcctgggtcgactaagtgatatcctgacctaactgcagagatcagttgccctacatctgccagtttgccattgtggcggccgcaggtgcgccggtgccgtatccggatccgctggaaccgcgtgccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 164 - DNA, 4101 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctaagtgacgatatcctgacctaaggtacctaagtgacgatatcctgacctaactgcagggatcaattgccctacatctgccagttcgggatcgtggcggccgcaggtgcgccggtgccgtatccggatccgctggaaccgcgtgccgcacaggctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 165 - DNA, 4114 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccttacagactgtgtgcctgaagggcaccaaggtgaacttgaagtgcctcctggccttcacccaaccgaagaccttccatgaggcgagcgaggactgcatctcgcaagggggcacgctgggtaccccgcagtcagagctggagaacgaggcgctgttcgaatacgcgcgccacagcgtgggcaacgatgcgaacatctggctgggcctcaacgacatggccgcggaaggcgcctgggtcgactaagtgatatcctgacctaactgcagagatcagttgccctacatctgccagtttgccattgtggcggccgcaggtgcgccggtgccgtatccggatccgctggaaccgcgtgccgcacaggctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

(19) United States
(12) Patent Application Publication
(10) Pub. No.: US 2011/0086806 A1
(43) Pub. Date: Apr. 14, 2011

(54) Polypeptides that Bind IL-23R

(75) Inventors: Anke Kretz-Rommel, San Diego, CA (US); Martha Wild, San Diego, CA (US); Katherine S. Bowdish, Del Mar, CA (US); Elise Chen, Del Mar, CA (US); Daniela Oltean, San Marcos, CA (US); Maria Gonzalez, Cardiff, CA (US); Mili Kapoor, San Diego, CA (US)

(73) Assignee: Anaphore, Inc.

(21) Appl. No.: 12/703,757

(22) Filed: Feb. 10, 2010

Related U.S. Application Data
(63) Continuation-in-part of application No. 12/703,752, filed on Feb. 10, 2010.
Application No. 12/703,752 is a continuation-in-part of application No. 12/577,067, filed on Oct. 9, 2009.

(30) Foreign Application Priority Data
Oct. 9, 2009 (US) PCT/US09/60271

(51) Int. Cl. A61K 38/17; C07H 21/04; C07K 14/47; C07K 19/00; A61P 35/00; A61P 29/00; C40B 30/04; C12N 15/63; C12N 5/10

(52) U.S. Cl. 514/19.3; 536/23.1; 530/350; 530/402; 506/9; 435/320.1; 435/325; 514/20.6

(57) ABSTRACT

Polypeptides that bind to IL-23R including polypeptides having a multimerizing, e.g. trimerizing, domain and a polypeptide sequence that binds IL-23R. The multimerizing domain may be derived from human tetranectin. IL-23R binding polypeptides inhibit activation of IL-23R by native IL-23 and can be used as therapeutics agents for a variety of immune related disorders and cancers. Methods for selecting polypeptides and preparing multimeric complexes are described.

### CROSS-REFERENCE TO RELATED APPLICATION

This application is a continuation-in-part of U.S. patent application Ser. No. 12/577,067, filed Oct. 9, 2009, a continuation-in-art of International Application PCTUS09/60271, filed Oct. 9, 2009, and a CIP of U.S. application Ser. No. 12/703,752, filed Feb. 10, 2010, each of which is incorporated by reference herein in its entirety.

### SEQUENCE LISTING STATEMENT

The sequence listing is filed in this application in electronic format only and is incorporated by reference herein. The sequence listing text file “10-090_Substitute_SeqList.txt” was created on Mar. 2, 2010, and is 390 kilobytes in size.

### FIELD OF THE INVENTION

The invention relates broadly to the treatment of inflammatory and autoimmune diseases as well as cancer. In particular, the invention relates to polypeptides that bind to the IL-23R subunit of the IL-23R heterodimeric receptor and that block interaction of IL-23 with its receptor.

### BACKGROUND OF THE INVENTION

IL-23 is an essential cytokine for generation and survival of Th17 cells. There is mounting evidence from preclinical models and clinical experience that Th17 cells play a critical role in pathology of many autoimmune diseases, including rheumatoid arthritis, inflammatory bowel disease, psoriasis, systemic lupus erythematosus (SLE) and multiple sclerosis. IL-23R is a key target on Th17 cells. The IL-23 heterodimeric receptor is composed of two subunits: IL-23R and IL-12Rβ1, with IL-23R being the subunit unique to the IL-23 pathway. IL-12Rβ1 is shared with the IL-12 receptor and hence the IL-12 pathway. Similarly, the IL-23 cytokine is composed of two subunits: p19 and p40, with the p19 subunit being unique to IL-23, and p40 shared with IL-12. Binding of IL-23 to the heterodimeric IL-23 receptor mediates activation of certain T cell subsets, NK cells and myeloid cells.

Importantly, genetic variation in IL-23R has been associated with susceptibility to psoriasis and Crohn's disease and also has been implicated in susceptibility to ankylosing spondylitis, Vogt-Koyanagi-Harada disease, Systemic Sclerosis, Behcet's disease (BD), Primary Sjögren's Syndrome, Goodpasture disease. Also, importance of IL-23 in Graft Versus Host disease and chronic ulcers has been suggested, and IL-23 has been implicated in tumorigenesis.

Blockade of the IL-23 pathway is efficacious in many preclinical models of autoimmune disease. However, the nature of shared ligand and receptor subunits between IL-23 and IL-12 pathways has led to more complex biology than previously appreciated, and separation of IL-23 blockade from IL-12 blockade appears to have important therapeutic implications regarding both efficacy and safety. Blockade of one or the other, or both, can be done at the level of the cytokine subunits or the receptor subunits.

While antibodies targeting the IL-23/IL-12 cytokines are approved (e.g., p40-targeted Ustekinumab) or in clinical development (Abbott Laboratories), along with Schering Plough's IL-23 specific anti-p19 antibody in early clinical development, there is a need for IL-23 specific blockade with superior efficacy and better safety profile for the following reasons:
The distribution of IL-23 heterodimeric receptor is relatively limited with IL-23 heterodimeric receptor expressing cells primarily found in inflamed/diseased tissue. In contrast, IL-23 can be detected systemically and is more abundant.
Targeting the receptor over the p19 subunit of IL-23 has been shown to be advantageous in situations where the cytokine is cell bound and/or not abundant as demonstrated in autoimmune tissues such as synovium from rheumatoid arthritis patients.
Targeting receptors will more efficiently block in patients with receptor variants that might be more susceptible to IL-23 signaling (i.e. low threshold variants where very little ligand is required for signaling).

Also, while originally developed to block IL-12, there is preclinical and clinical evidence that Ustekinumab's efficacy is mediated through IL-23 blockade, and that blocking the IL-12 pathway could be detrimental based on the following observations:
In psoriasis trials with Ustekinumab, p19, the IL-23-specific cytokine subunit (but not p35, the IL-12-specific cytokine subunit) was down-regulated in plaques.
While p19 and p40 knock-out mice are resistant to induction of experimental autoimmune disease, knock-out of the IL-12 specific subunit p35 exacerbated a number of experimental autoimmune diseases.
In addition to the potential for superior efficacy, selectively blocking IL-23 over both IL-12 and IL-23 has considerable advantages with regard to safety related to susceptibility to infections, as blocking both cytokines has been shown to increase susceptibility to Toxoplasma gondii, Cryptococcus neoformans, and M. tuberculosis , and likely other pathogens.
Safety advantages may also relate to the potential for tumorigenicity. Preclinical data suggest that inhibiting IL-12 enhances tumor growth while inhibiting IL-23 might reduce tumor growth. In contrast to IL-12p40, IL-23 is over-expressed in human tumors. Furthermore, murine validation studies demonstrate that IL-23 knockout mice, or anti-IL-23 treated mice, resist tumor formation, while elevated IL-23 levels can increase tumor formation.

Accordingly, there is a need in the art for molecules that selectively block the IL-23 heterodimeric receptor by blocking IL-23R, compositions comprising those molecules, methods for screening for such molecules, and methods for using such molecules in the therapeutic treatment of a wide variety of inflammatory and autoimmune conditions and cancer. Such molecules should demonstrate good target retention due to avidity effects, and should localize therapy to sites of inflammation associated with the disorder without significantly compromising systemic immunity.

### SUMMARY OF THE INVENTION

In one aspect, the invention is directed to a polypeptide having a trimerizing domain and at least one polypeptide sequence that binds to human IL-23R without activating IL-23 heterodimeric receptor. In other aspects, the polypeptide of the invention does not bind to at least one of human IL-12Rβ1 or human IL-12Rβ2, and the polypeptide competes with native human IL-23 for binding to human IL-23R. The trimerizing domain may include a polypeptide of a human tetranectin trimerizing domain (SEQ ID NO: 99) having up to five amino acid substitutions at positions 26, 30, 33, 36, 37, 40, 41, 42, 45, 46, 47, 48, 49, 50 and 51. These polypeptides can form a trimeric complex. The polypeptides may trimerize to form a trimeric complex.

Even further, the polypeptide of the invention includes at least one polypeptide that binds IL-23R and is linked to one of the N-terminus and the C-terminus of the trimerizing domain, and also includes a modulator of inflammation positioned at the other of the N-terminus and the C-terminus. The polypeptide of the invention may also have a polypeptide that binds IL-23 linked to each of the N-terminus and the C-terminus, wherein the polypeptide at the N-terminus is the same or different than the polypeptide at the C-terminus. The polypeptide may also have a therapeutic agent covalently attached to the polypeptide

Still further, the polypeptide of the invention includes a C-Type Lectin Like Domain (CLTD) and wherein one of loops 1, 2, 3 or 4 of loop segment A or loop segment B of the CTLD comprises a polypeptide sequence that binds IL-23. In various aspects the polypeptide sequence of the CTLD is selected from the group consisting of SEQ ID NO:133, 134, 135, 167, 137, 138, 139, 140, and 141.

The invention is also directed to a method of preventing activation of IL-23R by IL-23 in cells that express IL-23R. The method includes contacting the cell with the trimeric complex of the invention. In another aspect, the invention includes a pharmaceutical composition including the trimeric complex and at least one pharmaceutically acceptable excipient. The composition can be administered to treat an immune disorder or cancer. The composition may also include a modulator of inflation, a chemotherapeutic agent or a cytotoxic agent.

Still further, the invention is directed to method for preparing the polypeptide of the invention. The method includes selecting a first polypeptide that binds to IL-23R and fusing the first polypeptide with one of the N-terminus or the C-terminus of a multimerizing domain. The method may also include selecting a second polypeptide sequence that is a modulator of inflammation; and fusing the second polypeptide with the other of the N-terminus or the C-terminus of the multimerizing domain. The first polypeptide may be selected so that it does not bind to at least one of IL-12Rβ1 or IL-12Rβ2. The polypeptides can be used to prepare a trimeric complex that prevents activation of IL-23R in a cell expressing IL-23R.

Still further, the invention is directed to a polypeptide that competes with native human IL-23 for binding to native IL-23R, wherein the polypeptide does not activate human IL-23R and does not bind to at least one of IL-12Rβ1 or IL-12Rβ2. The polypeptide may be a CTLD that has been modified in one of loops 1, 2, 3 or 4 of loop segment A or in loop segment B for binding to IL-23R, and may be selected from one of SEQ ID NO:133, 134, 135, 136, 137, 138, 139, 140, and 141.

### DESCRIPTION OF THE FIGURES

FIGS. 1A and 1B show the polypeptide sequence of human IL-23 (SEQ ID NO: 1), human IL-23R (SEQ ID NO: 5), human IL-12Rβ1 (SEQ ID NO: 6), human IL-12Rβ2 (SEQ ID NO: 7), human IL-12A (SEQ ID NO: 3), and human IL-12B (SEQ ID NO: 2).

FIGS. 2A, B, C and D show examples of tetranectin trimerizing module variants for use with exemplary polypeptides of the invention.

FIG. 3 shows alignment of the amino acid sequences of the trimerising structural element of the tetranectin protein family. Amino acid sequences (one letter code) corresponding to residue V17 to K52 comprising exon 2 and the first three residues of exon 3 of human tetranectin (SEQ ID NO: 99); murine tetranectin (SEQ ID NO: 100) (Sorensen et al., Gene, 152: 243-245, 1995); tetranectin homologous protein isolated from reefshark cartilage (SEQ ID NO: 107) (Neame and Boynton, 1992, 1996); and tetranectin homologous protein isolated from bovine cartilage (SEQ ID NO: 106) (Neame and Boynton, database accession number PATCHX:u22298) are underlined. Residues at a and d positions in the heptad repeats are listed in boldface. The listed consensus sequence (SEQ ID NO: 108) of the tetranectin protein family trimerizing structural element comprise the residues present at a and d positions in the heptad repeats shown in the figure in addition to the other conserved residues of the region. “*” denotes an aliphatic hydrophobic residue.

FIG. 4 shows an alignment of the amino acid sequences of ten CTLDs of known 3D-structure. The sequence locations of main secondary structure elements are indicated above each sequence, labeled in sequential numerical order as “αN”, denoting a α-helix number N, and “βM”, denoting β-strand number M. The four cysteine residues involved in the formation of the two conserved disulfide bridges of CTLDs are indicated and enumerated in the Figure as “CI”, “CII”, “CIII” and “CIV” respectively. The two conserved disulfide bridges are CI-CIV and CII-CIII, respectively. The various loops 1-4 and LSB (loop 5) in the human tetranectin sequence are indicated by underlining. The ten C-type lectins are hTN: human tetranectin (SEQ ID NO: 109), MBP: mannose binding protein (SEQ ID NO: 110); SP-D: surfactant protein D (SEQ ID NO: 111); LY49A: NK receptor LY49A (SEQ ID NO: 112); H1-ASR: H1 subunit of the asialoglycoprotein receptor (SEQ ID NO: 113); MMR-4: macrophage mannose receptor domain 4 (SEQ ID NO: 114); IX-A (SEQ ID NO: 115) and IX-B (SEQ ID NO: 116): coagulation factors IX/X-binding protein domain A and B, respectively; Lit: lithostatine (SEQ ID NO: 117); TU14: tunicate C-type lectin (SEQ ID NO: 118). All of these CTLDs are from human proteins except TU14.

FIG. 5 depicts an alignment of the amino acid sequences of tetranectins isolated from human (Swissprot P05452) (SEQ ID NO: 119), mouse (Swissprot P43025) (SEQ ID NO: 120), chicken (Swissprot Q9DDD4) (SEQ ID NO: 121), bovine (Swissprot Q2KIS7) (SEQ ID NO: 122), Atlantic salmon (Swissprot B5XCV4) (SEQ ID NO: 123), frog (Swissprot Q510R9) (SEQ ID NO: 124), zebrafish (GenBank XP 701303) (SEQ ID NO: 125), and related CTLD homologues isolated from cartilage of cattle (Swissprot u22298) (SEQ ID NO: 126) and reef shark (Swissprot p26258) (SEQ ID NO: 127).

FIG. 6 shows the PCR strategy for creating randomized loops in a CTLD.

FIG. 7 shows the DNA and amino acid sequence of the human tetranectin CTLD modified to contain restriction sites for cloning, indicating the Ca2+ binding sites. Restriction sites are underscored with solid lines. Loops are underlined with dashed lines. Calcium coordinating residues are in bold italics and include Site 1: D116, E120, G147, E150, N151; Site 2: Q143, D145, E150, D165. The CTLD domain starts at amino acid A45 in bold (i.e. ALQTVCL . . . ). Changes to the native tetranectin (TNCTLD) base sequence are shown in lower case. The restriction sites were created using silent mutations that did not alter the native amino acid sequence.

FIG. 8 shows a number of sequences of polypeptides of the invention that bind to IL-23R. The sequences were produced according to the method of the invention by selecting polypeptides from a library of polypeptides having the scaffold structure of a human tetranectin CTLD that have been modified in one more loop regions. The CTLD scaffold of these sequences starts at A45 of human tetranectin (SEQ ID NO: 119). The portions of the sequence showing the loop regions that have been randomized are underlined.

FIG. 9 depicts an alignment of the nucleotide and amino acid sequences of the coding regions of the mature forms of human (SEQ ID NOS: 143 [nucleotide sequence] and 142 [amino acid sequence]) and murine tetranectin (SEQ ID NOS: 144 [nucleotide sequence] and 145 [amino acid sequence]) starting at their trimerizing domains, with an indication of known secondary structural elements.

FIG. 10 shows the results of a competition ELISA. Binding of human IL-23 to human IL-23R in the presence or absence of the polypeptides of the invention was evaluated.

FIG. 11 shows the results of an experiment comparing IL-23-induced IL-17 production in the presence of ATRIMER™ complex 4G8 of the invention, native human IL-23, and Ustekinumab.

FIG. 12 shows the results of an experiment comparing IL-23-induced IL-17 production in the presence of ATRIMER™ complex 1A4 of the invention and Ustekinumab.

FIG. 13 shows the results of an experiment comparing IL-12-induced IFNγ production in the presence of the ATRIMER™ complex 4G8 of the invention, native human IL-23, and Ustekinumab.

FIG. 14 shows the results of an experiment comparing Stat-3 phosphorylation in NKL cell in response to IL-23 and the polypeptides of the invention.

FIG. 15 is a table showing experimental results associated with several ATRIMER™ polypeptide complexes of the invention.

FIG. 16 depicts the three dimensional structure (ribbon format) for human tetranectin, depicting the secondary structural features of the protein. The structure was solved in the Ca²⁺-bound form.

FIG. 17A depicts the three dimensional overlay structures of the CTLDs for human tetranectin (HTN) and several tetranectin homologues, including human mannose binding protein (MBP), rat mannose binding protein-C (MBP-C), human surfactant protein D, rat mannose binding protein-A (MBP-A), and rat surfactant protein A. The CTLD overlay structures were generated using Swiss PDB Viewer DeepView v. 4.0.1 for MacIntosh using the three-dimensional structure of human tetranectin as a template. FIG. 17B shows the corresponding amino acid sequences of the CTLDS for human tetranectin and the tetranectin homologues depicted in FIG. 17A. In FIG. 17B, 1HUP=human mannose binding protein, 1BV4A=rat mannose binding protein, 2GGUA=human surfactant protein D, 1KXOA=rat mannose binding protein A, 1R13=rat surfactant protein A.

FIG. 18A depicts the three dimensional overlay structures of the CTLDs for human tetranectin (HTN) and several tetranectin homologues, including human pancreatitis-associated protein, human dendritic cell-specific ICAM-3-grabbing non-integrin 2 (DC-SIGNR), rat aggrecan, mouse scavenger receptor, and human scavenger receptor. The CTLD overlay structures were generated using Swiss PDB Viewer DeepView v. 4.0.1 for MacIntosh using the three-dimensional structure of human tetranectin as a template. FIG. 18B shows the corresponding amino acid sequences of the CTLDS for human tetranectin and the tetranectin homologues depicted in FIG. 18A. In FIG. 18B, 1TDQB=rat aggrecan, 1UV0A=human pancreatitis-associated protein, 2OX8A=human scavenger receptor, 2OX9A=mouse scavenger receptor, and 1SL6A=human DC-SIGNR)

### DETAILED DESCRIPTION OF THE INVENTION

In various aspects, the invention is directed to polypeptides that bind IL-23R and that include polypeptide sequences of a multimerizing domain and one or more polypeptide sequences that bind to IL-23R. In one aspect the polypeptides of the invention function as IL-23R antagonists. Two, three, or more of the polypeptides can multimerize to form a multimeric complex including the polypeptides that bind IL-23R. In an alternative embodiment, the polypeptide binds IL-23R, but does not bind IL-12Rβ1 or IL-12β2. In addition, the invention provides methods for treating immune mediated disorders, cancer and other diseases in a subject by administering the polypeptide or multimeric complexes of the polypeptide to a patient in need.

### DEFINITIONS

Before defining the invention in further detail, a number of terms are defined. Unless a particular definition for a term is provided herein, the terms and phrases used throughout this disclosure should be taken to have the meaning as commonly understood in the art. Also, as used in this specification and the appended claims, the singular forms “a,” “an,” and “the” include plural referents unless the context clearly dictates otherwise.

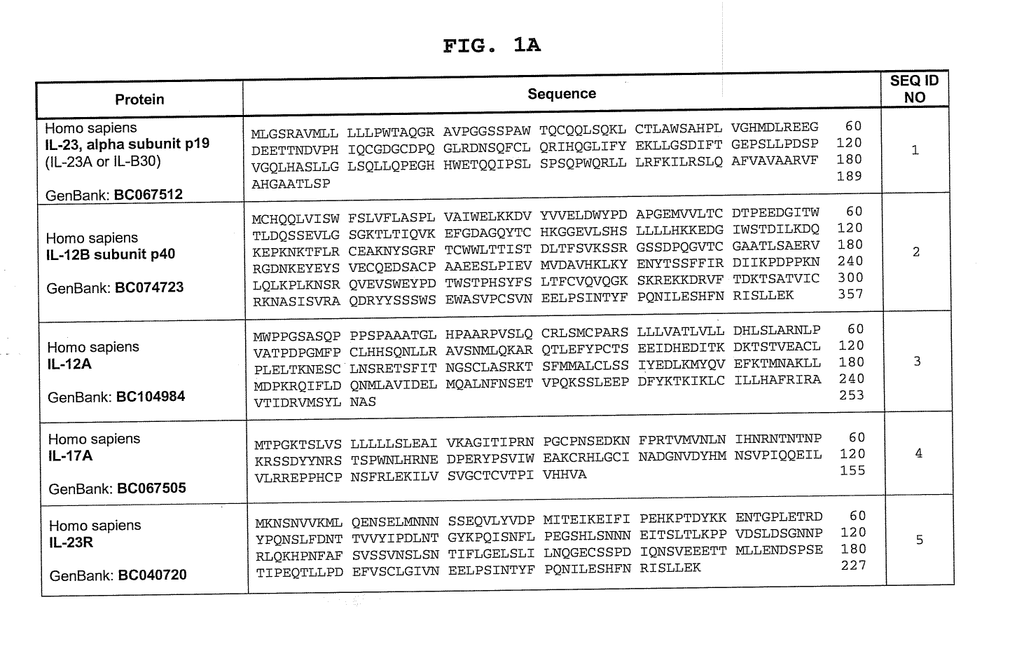

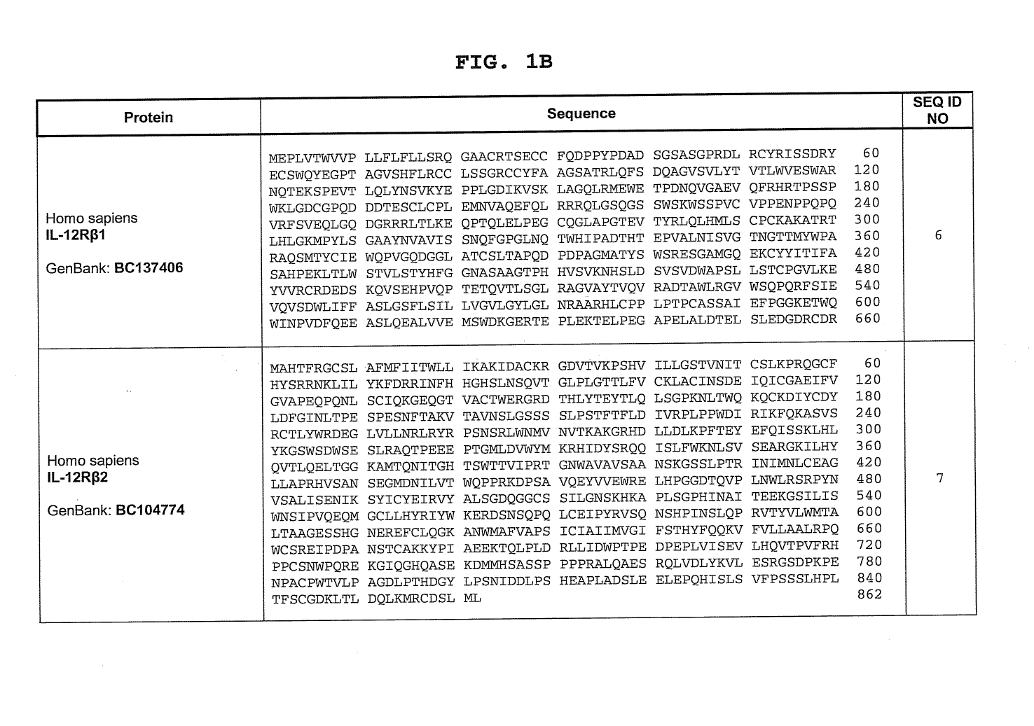

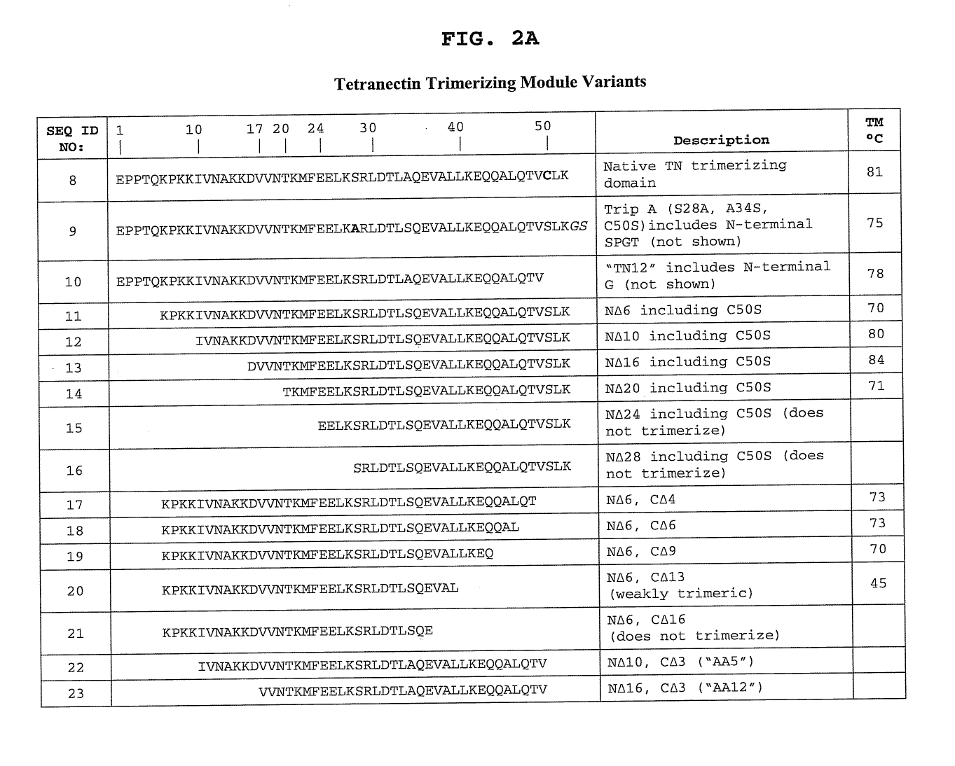

“IL-23” is a cytokine that functions in innate and adaptive immunity and refers to a hetero-dimeric protein complex belonging to the IL-6 superfamily. The heterodimeric complex is secreted by activated dendritic and phagocytic cells and keratinocytes. IL-23 is also expressed by dermal Langerhans cells. IL-23A, also known as IL-B30, the p19 subunit, or simply “p19,” associates with IL-12B, the p40 subunit, to form IL-23 (p19/p40). The amino acid sequences of IL-23A (p19) (SEQ ID NO: 1) and IL-12B (SEQ ID NO: 2) are shown in FIG. 1.

IL-23 is up-regulated by a wide array of pathogens and pathogen-products together with self-signals for danger or injury. IL-23 is up-regulated in psoriatic dermal tissues, in dendritic cells of multiple sclerosis patients and it has as well been shown that IL-23 is active in promoting tumor incidence and growth. In addition, IL-23 not only stimulates neutrophil and macrophage infiltration, but also promotes angiogenesis and inflammatory mediators in the tumor microenvironment. IL-23 can result in down-regulation of IL-12 and interferon γ, both of which are essential cytokines for cytotoxic immune responses, and controls the influx and activity of anti-tumor effector lymphocytes. It has been suggested that IL-23 inflicts a repurposing of the adaptive cytotoxic effector response away from anti-tumor immunity and towards proinflammatory and proangiogenic effector pathways that nourish the tumor. Consequently, IL-23 enables the persistence of the recognized tumor cells, accompanied by tumor-associated inflammation. This concept can explain tumor growth in the presence of large quantities of tumor-specific T cells.

The term “IL-23 heterodimeric receptor” refers to the heterodimeric polypeptide complex of IL-23R and IL-12Rβ1. This receptor binds IL-23. The polypeptide sequence of IL-23R and IL-12Rβ1 are shown in FIG. 1.

The term “IL-23R” refers to a polypeptide that can complex with IL-12Rβ1 to form the IL-23 heterodimeric receptor. IL-23R is also referred to as the IL-23R subunit.

The term “IL-12Rβ1” refers to the polypeptide that complexes with IL-23R to form the IL-23 heterotrimeric receptor and separately and independently with IL-12Rβ2 to form a heterodimeric IL-12 receptor. The polypeptide sequences of IL-12Rβ1 and IL-12Rβ2 are shown in FIG. 1.

“Inhibitors” and “antagonists” or “activators” and “agonists” refer to inhibitory or activating molecules, respectively. “Inhibitors” are compounds that decrease, block, prevent, delay activation, inactivate, desensitize, or down regulate biological function or activity associated with, for example, a gene, protein, ligand, receptor, or cell. Activators are compounds that increase, activate, facilitate, enhance activation, sensitize, or up regulate the biological function or activity of, for example, gene, protein, ligand, receptor, or cell. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

A “modulator” of a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, for example, a protein, metal ion, or small molecule.

The term “IL-23R antagonist” refers to any molecule that binds to IL-23R either alone or in complex with IL-12Rβ1 and blocks or dampens receptor signaling through a variety of mechanisms which can include blocking the ability of IL-23 to bind, blocking receptor heterodimer formation, or blocking or inducing changes that affect intracellular signaling, including conformational changes or receptor internalization.

The term “binding member” as used herein refers to a member of a pair of molecules which have binding specificity for one another. The members of a binding pair may be naturally derived or wholly or partially synthetically produced. One member of the pair of molecules has an area on its surface, or a cavity, which binds to and is therefore complementary to a particular spatial and polar organization of the other member of the pair of molecules. Thus the members of the pair have the property of binding specifically to each other.

“Specifically” or “selectively” binds, when referring to a ligand/receptor, antibody/antigen, or other binding pair, indicates a binding reaction which is determinative of the presence of member of a binding pair in a heterogeneous population of another member of the binding pair. Thus, under designated conditions, for example, a specified ligand binds to a particular receptor and does not bind in a significant amount to other proteins present in the sample.

As used herein, the term “multimerizing domain” means an amino acid sequence that comprises the functionality that can associate with other amino acid sequence(s) having a multimerizing domain to form multimeric complexes. In various embodiments of the invention, the multimerizing domain is a dimerizing domain, a trimerizing domain, a tetramerizing domain, a pentamerizing domain, etc. These domains are capable of forming polypeptide complexes of two, three, four, five or more polypeptides of the invention. In one example, the polypeptide contains an amino acid sequence—a “trimerizing domain”—which forms a trimeric complex with two other trimerizing domains. A trimerizing domain can associate with other trimerizing domains of identical amino acid sequence (a homotrimer), or with trimerizing domains of different amino acid sequence (a heterotrimer). Such an interaction may be caused by covalent bonds between the components of the trimerizing domains as well as by hydrogen bond forces, hydrophobic forces, van der Waals forces and salt bridges.

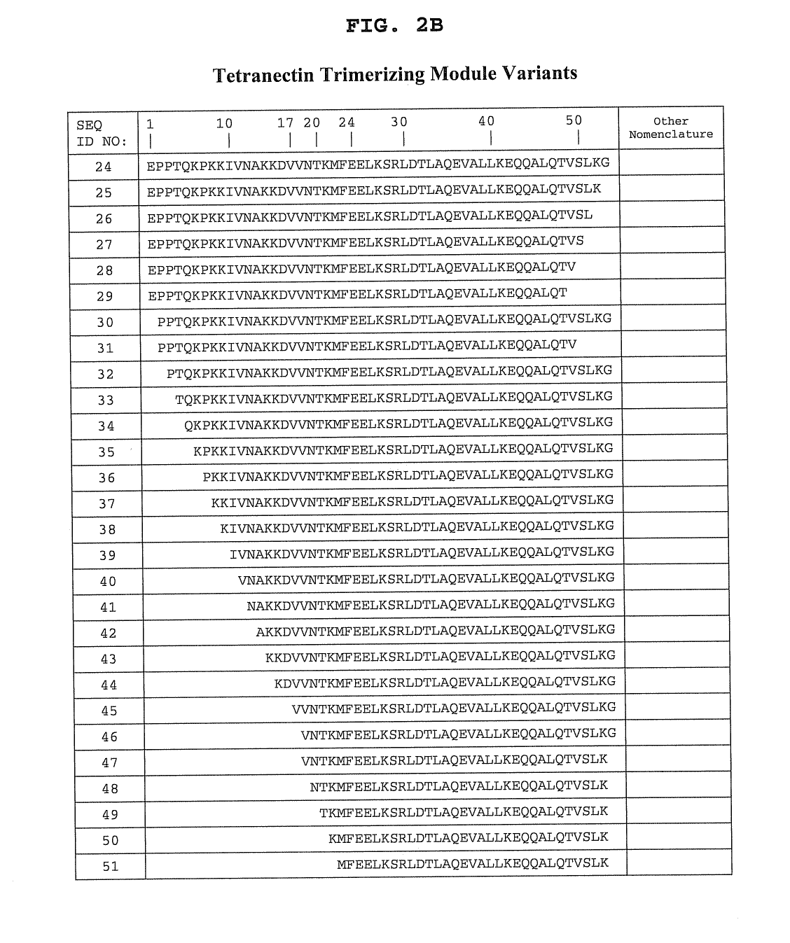

The trimerizing domain of a polypeptide of the invention may be derived from tetranectin as described in U.S. Patent Application Publication No. 2007/0154901 ('901 application), which is incorporated by reference in its entirety. The mature human tetranectin single chain polypeptide sequence is provided herein as SEQ ID NO: 142. Examples of a tetranectin trimerizing domain includes the amino acids 17 to 49, 17 to 50, 17 to 51 and 17-52 of SEQ ID NO: 99, which represent the amino acids encoded by exon 2 of the human tetranectin gene, and optionally the first one, two or three amino acids encoded by exon 3 of the gene. Other examples include amino acids 1 to 49, 1 to 50, 1 to 51 and 1 to 52, which represents all of exons 1 and 2, and optionally the first one, two or three amino acids encoded by exon 3 of the gene. Alternatively, only a part of the amino acid sequence encoded by exon 1 is included in the trimerizing domain. In particular, the N-terminus of the trimerizing domain may begin at any of residues 1, 2, 3, 4, 5, 6, 7, 8, 9, 10, 11, 12, 13, 14, 15, 16 and 17 of SEQ ID NO: 99. In particular embodiments, the N terminus is 110 or V17 and the C-terminus is Q47, T48, V49, C(S)50, L51 or K52 (numbering according to SEQ ID NO: 99). In addition, FIGS. 2A-2D provide a number of potential truncation variant of the human tetranectin trimerizing domain.

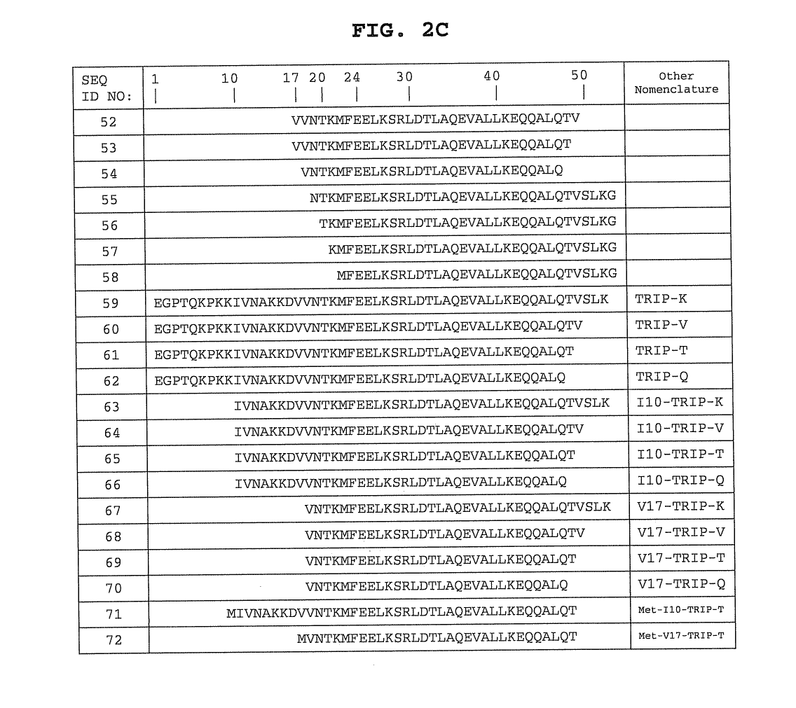

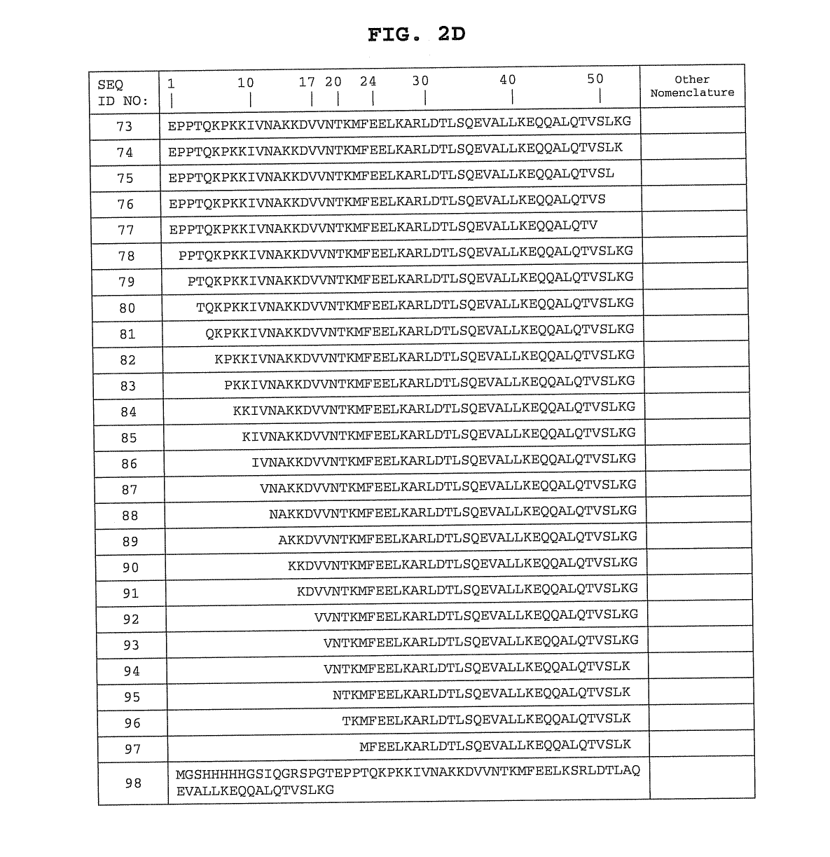

In one aspect of the invention, the trimerizing domain is a tetranectin trimerizing structural element (“TTSE”) having a amino acid sequence of SEQ ID NO: 108 which is a consensus sequence of the tetranectin family trimerizing structural element as more fully described in US 2007/00154901, which is incorporated herein by reference in its entirety. As shown in FIG. 3, the TTSE embraces variants of a naturally occurring member of the tetranectin family of proteins, and in particular variants that have been modified in the amino acid sequence without adversely affecting, to any substantial degree, the ability of the TTSE to form alpha helical coiled coil trimers. In various aspects of the invention, the trimeric polypeptide according to the invention includes a TTSE as a trimerizing domain having at least 66% amino acid sequence identity to the consensus sequence of SEQ ID NO: 108; for example at least 73%, at least 80%, at least 86% or at least 92% sequence identity to the consensus sequence of SEQ ID NO: 108 (counting only the defined (not X) residues). In other words, at least one, at least two, at least three, at least four, or at least five of the defined amino acids in SEQ ID NO: 108 may be substituted.

In one particular embodiment, the cysteine at position 50 (C50) of SEQ ID NO: 142 can be advantageously be mutagenized to serine, threonine, methionine or to any other amino acid residue in order to avoid formation of an unwanted inter-chain disulphide bridge, which can lead to unwanted multimerization. Other known variants include at least one amino acid residue selected from amino acid residue nos. 6, 21, 22, 24, 25, 27, 28, 31, 32, 35, 39, 41, and 42 (numbering according to SEQ ID NO: 142), which may be substituted by any non-helix breaking amino acid residue. These residues have been shown not to be directly involved in the intermolecular interactions that stabilize the trimeric complex between three TTSEs of native tetranectin monomers. In one aspect shown in FIG. 3, the TTSE has a repeated heptad having the formula a-b-c-d-e-f-g (N to C), wherein residues a and d (i.e., positions 26, 30, 33, 37, 40, 44, 47, and 51 may be any hydrophobic amino acid (numbering according to SEQ ID NO: 99).

In further embodiments, the TTSE trimerization domain may be modified by the incorporation of polyhistidine sequence and/or a protease cleavage site, e.g., Blood Coagulating Factor Xa or Granzyme B (see US 2005/0199251, which is incorporated herein by reference), and by including a C-terminal KG or KGS sequence. Also, to assist in purification, Proline at position 2 may be substituted with Glycine.

Particular non-limiting examples of TTSE truncations and variants are shown in FIGS. 2A-2D. In addition, a number of trimerizing domains having substantial homology (greater than 66%) to the trimerizing domain of human tetranectin known:

TABLE 1

| | | |
|---|---|---|
| Equus caballus TN-like | KMFEELKSQLDSLAQEVALLKEQQALQTVCL | SEQ ID NO: 146 |
| Cat TN | KMFEELKSQVDSLAQEVALLKEQQALQTVCL | SEQ ID NO: 147 |
| Mouse TN | SKMFEELKNRMDVLAQEVALLKEKQALQTVCL | SEQ ID NO: 148 |
| Rat TN | KMFEELKNRLDVLAQEVALLKEKQALQTVCL | SEQ ID NO: 149 |
| Bovine TN | KMLEELKTQLDSLAQEVALLKEQQALQTVCL | SEQ ID NO: 166 |
| Equus caballus CTLD | DLKTQVEKLWREVNALKEMQALQTVCL | SEQ ID NO: 167 |
| like | | |
| Canis lupus CTLD | DLKTQVEKLWREVNALKEMQALQTVCL | SEQ ID NO: 168 |
| member A | | |
| Bovine CTLD member A | DLKTQVEKLWREVNALKEMQALQTVCL | SEQ ID NO: 169 |
| Macaca mulatta CTLD | DLKTQIEKLWTEVNALKEIQALQTVCL | SEQ ID NO: 170 |
| member A | | |
| Taeniopygia guttata | DDLKTQIDKLWREVNALKEIQALQTVCL | SEQ ID NO: 171 |
| CTLD member A | | |
| Ornithorhynchus | DLKTQVEKLWREVNALKEMQALQTVCL | SEQ ID NO: 172 |
| anatinus CTLD like | | |
| Rat CTLD member A | DLKSQVEKLWREVNALKEMQALQTVCL | SEQ ID NO: 173 |
| Monodelphis domestics | DLKTQVEKLWREVNALKEMQALQTVCL | |
| CTLD member A | | |
| Shark TN | DDLRNEIDKLWREVNSLKEMQALQTVCL | SEQ ID NO: 175 |
| Taeniopygia guttata | KMIEDLKAMIDNISQEVALLKEKOALQTVCL | SEQ ID NO: 176 |
| TN-like | | |
| Gallus gallus TN | KMIEDLKAMIDNISQEVALLKEKQALQTVCL | SEQ ID NO: 177 |
| Danio rerio CTLD | DDMKTQIDKLWQEVNSLKEMQALQTVCL | SEQ ID NO: 178 |
| member A | | |
| Gallus gallus, CTLD | DDLKTQIDKLWREVNALKEMQALQSVCL | SEQ ID NO: 179 |
| member A | | |
| Mouse CTLD member A | DDLKSQVEKLWREVNALKEMQALQTVCL | SEQ ID NO: 180 |
| Gallus gallus CTLD | DDLKTQIDKLWREVNALKEMQALQSVCL | SEQ ID NO: 181 |
| member A | | |
| Tetraodon | DDVRSQIEKLWQEVNSLKEMQALQTVCL | SEQ ID NO: 182 |
| nigroviridis, unknown | | |
| Xenopus laevis | DLKTQIDKLWREINSLKEMQALQTVCL | SEQ ID NO: 183 |
| MGC85438 | | |
| Tetraodon | EELRRQVSDLAQELNILKEQQALHTVCL | SEQ ID NO: 184 |
| nigroviridis, unknown | | |
| Xenopus laevis, unknown | KMYEELKQKVQNIELEVIHLKEQQALQTICL | SEQ ID NO: 185 |
| Xenopus tropicalis TN | KMYEDLKKKVQNIEEDVIHLKEQQALQTICL | SEQ ID NO: 186 |
| Salmo salar TN | EELKKQIDNIVLELNLLKEQQALQSVCL | SEQ ID NO: 187 |
| Danio rerio TN | EELKKQIDQIIQDLNLLKEQQALQTVCL | SEQ ID NO: 188 |
| Tetraodon | EQMQKQINDIVQELNLLKEQQALQAVCL | SEQ ID NO: 189 |
| nigroviridis, unknown | | |
| Tetraodon | EQMQKQINDIVQELNLLKEQQALQAVCL | SEQ ID NO: 190 |
| nigroviridis, unknown | | |

Other human polypeptides that are known to trimerize include:

| | | |
|---|---|---|
| hTRAF3 | NTGLLESQLSRHDQMLSVHDIRLADMDLRFQVLETASYNG | SEQ ID NO: 191 |
| | VLIWKIRDYKRRKQEAVM | |
| hMBP | AASERKALQTEMARIKKWLTF | SEQ ID NO: 192 |
| hSPC300 | FDMSCRSRLATLNEKLTALERRIEYIEARVTKGETLT | SEQ ID NO: 193 |
| hNEMO | ADIYKADFQAERQAREKLAEKKELLQEQLEQLQREYSKLK | SEQ ID NO: 194 |
| | ASCQESARI | |
| hcubilin | LTGSAQNIEFRTGSLGKIKLNDEDLSECLHQIQKNKEDII | SEQ ID NO: 195 |
| | ELKGSAIGLPIYQLNSKLVDLERKFQGLQQT | |
| hThrombos | LRGLRTIVTTLQDSIRKVTEENKELANE | SEQ ID NO: 196 |
| pondins | | |

Another example of a trimerizing domain is disclosed in U.S. Pat. No. 6,190,886 (incorporated by reference herein in its entirety), which describes polypeptides comprising a collectin neck region. Trimers can then be made under appropriate conditions with three polypeptides comprising the collectin neck region amino acid sequence. A number of collectins are identified, including:

Collectin neck region of human SP-D:

| | |
|---|---|
| VASLRQQVEALQGQVQHLQAAFSQYKK | [SEQ ID NO: 197] |

Collectin neck region of bovine SP-D:

| | |
|---|---|
| VNALRQRVGILEGQLQRLQNAFSQYKK | [SEQ ID NO: 198] |

Collectin neck region of rat SP-D:

| | |
|---|---|
| SAALRQQMEALNGKLQRLEAAFSRYKK | [SEQ ID NO: 199] |

Collectin neck region of bovine conglutinin:

| | |
|---|---|
| VNALKQRVTILDGHLRRFQNAFSQYKK | [SEQ ID NO: 200] |

Collectin neck region of bovine collectin:

| | |
|---|---|
| VDTLRQRMRNLEGEVQRLQNIVTQYRK | [SEQ ID NO: 201] |

Neck region of human SP-D:

[SEQ ID NO: 202]
GSPGLKGDKGIPGDKGAKGESGLPDVASLRQQVEALQGQVQHLQAAFSQY
KKVELFPGGIPHRD

Other examples of a MBP trimerizing domain is described in PCT Application Serial No. US08/76266, published as WO 2009/036349, which is incorporated by reference in its entirety. This trimerizing domain can oligomerize even further and create higher order multimeric complexes.

In the present context, the “trimerising domain” is capable of interacting with other, similar or identical trimerising domains. The interaction is of the type that produces trimeric proteins or polypeptides. Such an interaction may be caused by covalent bonds between the components of the trimerising domains as well as by hydrogen bond forces, hydrophobic forces, van der Waals forces, and salt bridges. The trimerising effect of trimerizing domain is caused by a coiled coil structure that interacts with the coiled coil structure of two other trimerizing domains to form a triple alpha helical coiled coil trimer that is stable even at relatively high temperatures. In various embodiments, for example a trimerizing domain based upon a tetranectin structural element, the complex is stable at least 60° C., for example in some embodiments at least 70° C.

The terms “C-type lectin-like protein” and “C-type lectin” are used to refer to any protein present in, or encoded in the genomes of, any eukaryotic species, which protein contains one or more CTLDs or one or more domains belonging to a subgroup of CTLDs, the CRDs, which bind carbohydrate ligands. The definition specifically includes membrane attached C-type lectin-like proteins and C-type lectins, “soluble” C-type lectin-like proteins and C-type lectins lacking a functional transmembrane domain and variant C-type lectin-like proteins and C-type lectins in which one or more amino acid residues have been altered in vivo by glycosylation or any other post-synthetic modification, as well as any product that is obtained by chemical modification of C-type lectin-like proteins and C-type lectins.

The CTLD consists of roughly 120 amino acid residues and, characteristically, contains two or three intra-chain disulfide bridges. Although the similarity at the amino acid sequence level between CTLDs from different proteins is relatively low, the 3D-structures of a number of CTLDs have been found to be highly conserved, with the structural variability essentially confined to a so-called loop-region, often defined by up to five loops. Several CTLDs contain either one or two binding sites for calcium and most of the side chains which interact with calcium are located in the loop-region.

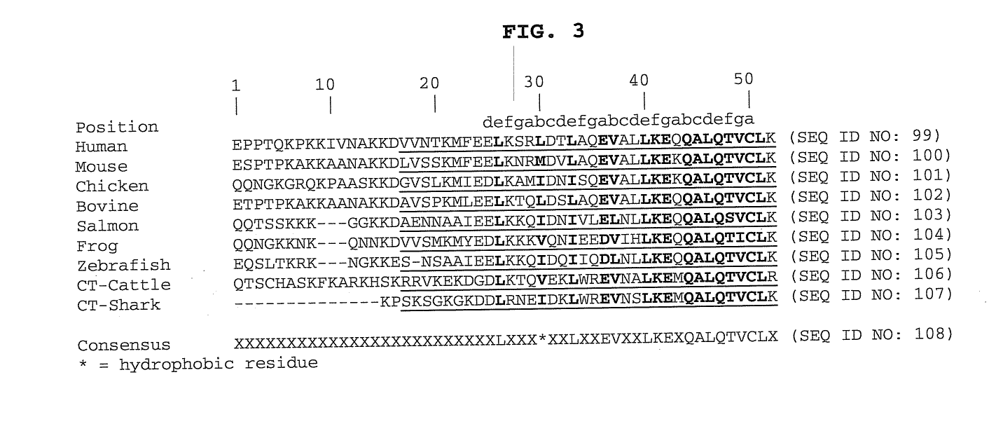

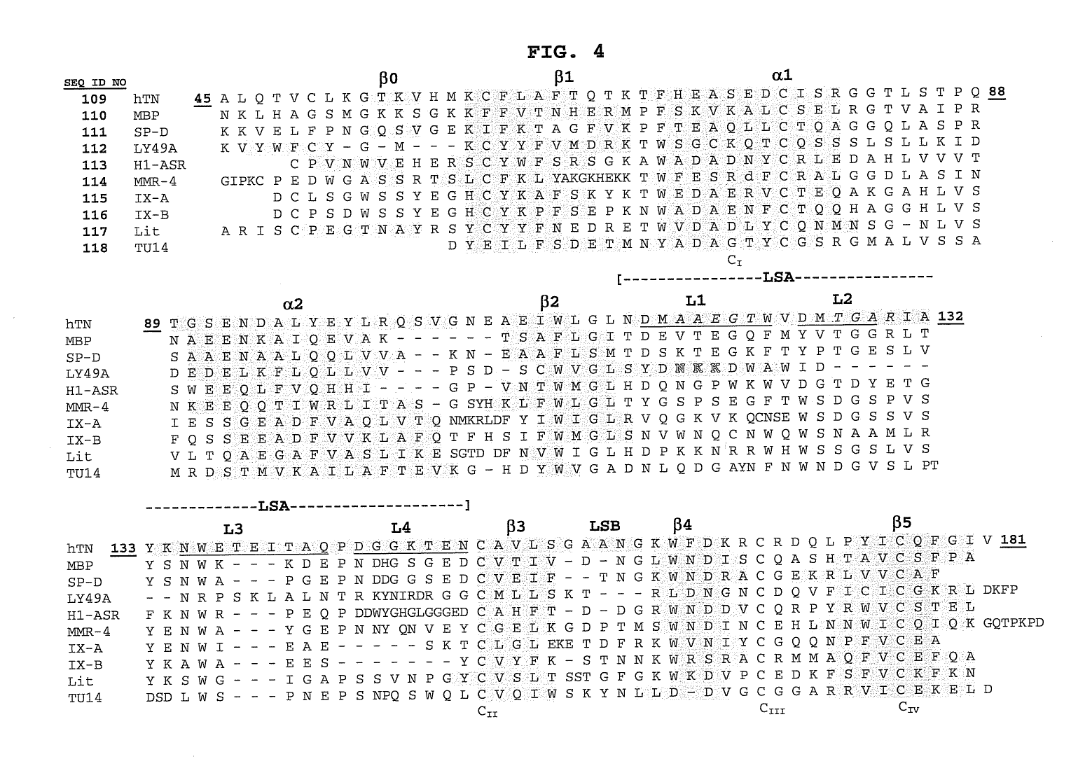

On the basis of CTLDs for which 3D structural information is available, it has been inferred that the canonical CTLD is structurally characterized by seven main secondary-structure elements (i.e. five β-strands and two α-helices) sequentially appearing in the order β1, α1, α2, β2, β3, β4, and β5. FIG. 4 illustrates an alignment of the CTLDs of ten known C-type lectins. In all CTLDs, for which 3D structures have been determined, the β-strands are arranged in two anti-parallel β-sheets, one composed of β1 and β5, the other composed of β2, β3 and β4. An additional β-strand, β0, often precedes β1 in the sequence and, where present, forms an additional strand integrating with the β1, β5-sheet. Further, two disulfide bridges, one connecting α1 and β5 (C_{I}-C_{IV}) and one connecting β3 and the polypeptide segment connecting β4 and β5 (C_{II}-C_{III}) are invariantly found in all CTLDs characterized to date. Also, FIG. 5 shows an alignment of CTLDs from human tetranectin and eight other tetranectin or tetranectin like polypeptides.

In the CTLD 3D-structure, these conserved secondary structure elements form a compact scaffold for a number of loops, which in the present context collectively are referred to as the “loop-region”, protruding out from the core. In the primary structure of the CTLDs, these loops are organized in two segments, loop segment A, LSA, and loop segment B, LSB. LSA represents the long polypeptide segment connecting β2 and β3 that often lacks regular secondary structure and contains up to four loops. LSB represents the polypeptide segment connecting the β-strands β3 and β4. Residues in LSA, together with single residues in β4, have been shown to specify the Ca²⁺- and ligand-binding sites of several CTLDs, including that of tetranectin. for example, mutagenesis studies, involving substitution of one or a few residues, have shown that changes in binding specificity, Ca²⁺-sensitivity and/or affinity can be accommodated by CTLD domains. A number of CLTDs are known, including the following non-limiting examples: tetranectin, lithostatin, mouse macrophage galactose lectin, Kupffer cell receptor, chicken neurocan, perlucin, asialoglycoprotein receptor, cartilage proteoglycan core protein, IgE Fc receptor, pancreatitis-associated protein, mouse macrophage receptor, Natural Killer group, stem cell growth factor, factor IX/X binding protein, mannose binding protein, bovine conglutinin, bovine CL43, collectin liver 1, surfactant protein A, surfactant protein D, e-selectin, tunicate c-type lectin, CD94 NK receptor domain, LY49A NK receptor domain, chicken hepatic lectin, trout c-type lectin, HIV gp120-binding c-type lectin, and dendritic cell immunoreceptor. See U.S. Patent Publication No. 2007/0275393, which is incorporated herein by reference in its entirety, and Essentials of Glycobiology, second edition. Edited by A. Varki, R. D. Cummings, J. D. Esko, H H. Freeze, P. Stanley, C. R. Bertozzi, G. W. Hart, M. E. Etzler. CHS Press.

An “ATRIMER™ polypeptide complex” or “ATRIMER™ complex” refers to a trimeric complex of three trimerizing domains that also include CLTDs (Anaphore, Inc., San Diego, Calif.).

The expression “effective amount” refers to an amount of a polypeptide of the invention, optionally in conjunction with a therapeutic agent which is effective for preventing, ameliorating or treating the disease or condition in question whether administered simultaneously or sequentially. In particular embodiments, an effective amount is the amount of the polypeptide of the invention, and a therapeutic agent, such as a cytotoxic or immunosuppressive agent, in combination sufficient to decrease the effects of IL-23 on IL-23R expressing cells, affect other pathways on IL-23R expressing cells working synergistically with IL-23R, or affecting other immune cells acting in concert with IL-23R expressing cells, decrease the propensity of a cell to proliferate or survive, or to enhance, or otherwise increase the propensity (such as synergistically) of a cell to undergo apoptosis, reduce tumor volume, or prolong survival of a mammal having a cancer or immune related disease.

A “therapeutic agent” refers to a cytotoxic agent, a chemotherapeutic agent, an immunosuppressive agent, an anti-inflammatory agent, an immunostimulatory agent, and/or a growth inhibitory agent.

The term “immunosuppressive agent” and “modulators of inflammation” as used herein for adjunct therapy refers to substances that act to suppress or mask the immune system of the mammal being treated herein. This would include substances that suppress cytokine production, downregulate or suppress self-antigen expression, inhibit migration of immune cells to sites of chronic inflammation, or mask the MHC antigens. Examples of such agents include but are not limited to 2-amino-6-aryl-5-substituted pyrimidines (see U.S. Pat. No. 4,665,077); nonsteroidal anti-inflammatory drugs (NSAIDs); azathioprine; cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, dexamethasone, and hydrocortisone; methotrexate (oral or subcutaneous); hydroxycloroquine; sulfasalazine; leflunomide; cytokine or cytokine receptor antagonists including anti-interferon-gamma (IFN-γ), -β, or -α antibodies, anti-tumor necrosis factor-α antibodies (such as e.g. infliximab, adalimumab or Cimzia), anti-TNFα immunoadhesin (etanercept), anti-tumor necrosis factor-β antibodies, anti-TGF-β antibodies, anti-interleukin-2 antibodies and anti-IL-2 receptor antibodies; anti-IL-6 antibodies, anti-IL-6R antibodies, anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodornase; RNA or DNA from the host; FK506; RS-61443; deoxyspergualin; rapamycin; T-cell receptor (Cohen et al., U.S. Pat. No. 5,114,721); T-cell receptor fragments (Offner et al., Science, 251: 430-432 (1991); WO 90/11294; Janeway, Nature, 341: 482 (1989); and WO 91/01133); and T-cell receptor antibodies (EP 340,109) such as T10B9, integrin inhibitors such as Tysabri, CCR9 or CCR6 antagonists, anti-TL1A antibodies or cytokines known to suppress immune responses such as IL-10 or IL-27.

The term “cytotoxic agent” as used herein refers to a substance that inhibits or prevents the function of cells and/or causes destruction of cells. The term is intended to include radioactive isotopes (e.g. At²¹¹, I¹³¹I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma 1l and calicheamicin omega 1l (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,22″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition are proteasome inhibitors such as bortezomib (Velcade), BCL-2 inhibitors, IAP antagonists (e.g. Smac mimics/xIAP and cIAP inhibitors such as certain peptides, pyridine compounds such as (S)-N-{6-benzo[1,3]dioxol-5-yl-1-[5-(4-fluoro-benzoyl)-pyridin-3-ylmethyl]-2-oxo-1,2-dihydro-pyridin-3-yl}-2-methylamino-propionamide, xIAP antisense), HDAC inhibitors (HDACI) and kinase inhibitors (Sorafenib).

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell, either in vitro or in vivo. Thus, the growth inhibitory agent is one that significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and top( ) II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995, pg. 13).

Further included are agents that induce cell stress such as e.g. arginine depleting agents such as arginase.

Further included are antibodies affecting B cells such as Rituximab, anti-BAFF or anti-APRIL antibodies and T cell depleting antibodies such as Campath. Furthermore, combinations of IL-23R antagnoists with aspirin and inhibitors of the NFkB pathway can be beneficial.

“Synergistic activity,” “synergy,” “synergistic effect,” or “synergistic effective amount” as used herein means that the effect observed when employing a combination of an IL-23R antagonist and a therapeutic agent is (1) greater than the effect achieved when that IL-23R antagonist or therapeutic agent is employed alone (or individually) and (2) greater than the sum added (additive) effect for that IL-23R antagonist or therapeutic agent. Such synergy or synergistic effect can be determined by way of a variety of means known to those in the art. For example, the synergistic effect of IL-23R antagonist and a therapeutic agent can be observed in in vitro or in vivo assay formats examining reduction in cytokine release from immune cells, number or type of immune cells present, or in the case of cancer, in reduction of tumor cell number or tumor mass.

The terms “cancer”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer (NSCLC), gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

The term “immune related disease” means a disease or disorder in which a component of the immune system of a mammal causes, mediates or otherwise contributes to morbidity in the mammal. Also included are diseases in which stimulation or intervention of the immune response has an ameliorative effect on progression of the disease. Included within this term are autoimmune diseases, immune-mediated inflammatory diseases. Examples of immune-related and inflammatory diseases, some of which are immune or T cell mediated, which can be treated according to the invention include systemic lupus erythematosis, rheumatoid arthritis, juvenile chronic arthritis, spondyloarthropathies, ankylosing spondylitis, systemic sclerosis (scleroderma), idiopathic inflammatory myopathies (dermatomyositis, polymyositis), primary Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barre syndrome, Vogt-Koyanagi-Harada disease, Goodpasture disease, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory diseases such as inflammatory bowel disease (ulcerative colitis: Crohn's disease), gluten-sensitive enteropathy, Whipple's disease, and fibrotic lung diseases, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease, immune-mediated or autoimmune eye diseases such as uveitis, dry eye, Behçcet's disease (BD).

Infectious diseases include AIDS (HIV infection), hepatitis A, B, C, D, and E, bacterial infections, fungal infections, protozoal infections and parasitic infections.

A “B-cell malignancy” is a malignancy involving B cells. Examples include Hodgkin's disease, including lymphocyte predominant Hodgkin's disease (LPHD); non-Hodgkin's lymphoma (NHL); follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLD); Waldenstrom's macroglobulinemia (lymphoplasmacytic lymphoma); mucosa-associated lymphoid tissue (MALT) lymphoma; and marginal zone lymphoma/leukemia.

“Non-Hodgkin's lymphoma” (NHL) includes, but is not limited to, low grade/follicular NHL, relapsed or refractory NHL, front line low grade NHL, Stage III/IV NHL, chemotherapy resistant NHL, small lymphocytic (SL) NHL, intermediate grade/follicular NHL, intermediate grade diffuse NHL, diffuse large cell lymphoma, aggressive NHL (including aggressive front-line NHL and aggressive relapsed NHL), NHL relapsing after or refractory to autologous stem cell transplantation, high grade immunoblastic NHL, high grade lymphoblastic NHL, high grade small non-cleaved cell NHL, bulky disease NHL, etc.

“Tumor-associated antigens” (TAA) or “tumor-specific antigens” (TSA) are molecules produced in tumor cells that can trigger an immune response in the host. Tumor associated antigens are found on both tumor and normal cells, although at differential expression levels, whereas tumor specific antigens are exclusively expressed by tumor cells. TAAs or TSAs exhibiting on the surface of tumor cells include but are not limited to alfafetoprotein, carcinoembryonic antigen (CEA), CA-125, MUC-1, glypican-3, tumor associated glycoprotein-72 (TAG-72), epithelial tumor antigen, tyrosinase, melanoma associated antigen, MART-1, gp100, TRP-1, TRP-2, MSH-1, MAGE-1, -2, -3, -12, RAGE-1, GAGE 1-, -2, BAGE, NY-ESO-1, beta-catenin, CDCP-1, CDC-27, SART-1, EpCAM, CD20, CD23, CD33, EGFR, HER-2, breast tumor-associated antigens BTA-1 and BTA-2, RCAS1 (receptor-binding cancer antigen expressed on SiSo cells), PLACenta-specific 1 (PLAC-1), syndecan, MN (gp250), idiotype, among others. Tumor associated antigens also include the blood group antigens, for example, Le^{a}, Le^{b}, LeX, LeY, H-2, B-1, B-2 antigens. (See Table 19 at the end of the specification). Ideally, for the purposes of this invention, TAA or TSA targets do not get internalized upon binding.

A “non-natural amino acid” or “non-naturally occurring amino acid” refers to an amino acid that is not one of the 20 common amino acids including, for example, amino acids that occur by modification (e.g. post-translational modifications) of a naturally encoded amino acid (including but not limited to, the 20 common amino acids or pyrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

“Conservatively modified variants” applies to both amino acid and nucleic acid sequences. With respect to particular nucleic acid sequences, conservatively modified variants refers to those nucleic acids which encode identical or essentially identical amino acid sequences or, where the nucleic acid does not encode an amino acid sequence, to essentially identical nucleic acid sequences. Because of the degeneracy of the genetic code, a large number of functionally identical nucleic acids may encode any given protein.

As to amino acid sequences, one of skill will recognize that an individual substitution to a nucleic acid, peptide, polypeptide, or protein sequence which substitutes an amino acid or a particular percentage of amino acids in the encoded sequence for a conserved amino acid is a “conservatively modified variant.” Conservative substitution tables providing functionally similar amino acids are well known in the art.

An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157: 105-132): (1) Hydrophobic: Norleucine, Ile, Val, Leu, Phe, Cys, or Met; (2) Neutral hydrophilic: Cys, Ser, Thr; (3) Acidic: Asp, Glu; (4) Basic: Asn, Gln, His, Lys, Arg; (5) Residues that influence chain orientation: Gly, Pro; (6) Aromatic: Trp, Tyr, Phe; (7) Small amino acids: Gly, Ala, Ser.

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100% Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicator of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, fluorescent dyes, electron-dense reagents, substrates, epitope tags, or enzymes, e.g., as used in enzyme-linked immunoassays, or fluorettes (see, e.g., Rozinov and Nolan (1998) Chem. Biol. 5:713-728).

Many of the unnatural amino acids suitable for use in the present invention are commercially available, e.g., from Sigma (USA) or Aldrich (Milwaukee, Wis., USA). Those that are not commercially available are optionally synthesized as provided herein or as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) A New Synthesis of Glutamine and of .gamma.-Dipeptides of Glutamic Acid from Phthylated Intermediates. J. Chem. Soc., 3315-3319; Friedman, O. M. & Chatterrji, R. (1959) Synthesis of Derivatives of Glutamine as Model Substrates for Anti-Tumor Agents. J. Am. Chem. Soc. 81, 3750-3752; Craig, J. C. et al. (1988) Absolute Configuration of the Enantiomers of 7-Chloro-4[[4-(diethylamino)-1-methylbutyl]amino]quinoline (Chloroquine). J. Org. Chem. 53, 1167-1170; Azoulay, M., Vilmont, M. & Frappier, F. (1991) Glutamine analogues as Potential Antimalarials, Eur. J. Med. Chem. 26, 201-5; Koskinen, A. M. P. & Rapoport, H. (1989) Synthesis of 4-Substituted Prolines as Conformationally Constrained Amino Acid Analogues. J. Org. Chem. 54, 1859-1866; Christie, B. D. & Rapoport, H. (1985) Synthesis of Optically Pure Pipecolates from L-Asparagine. Application to the Total Synthesis of (+)-Apovincamine through Amino Acid Decarbonylation and Iminium Ion Cyclization. J. Org. Chem. 1989: 1859-1866; Barton et al., (1987) Synthesis of Novel α-Amino-Acids and Derivatives Using Radical Chemistry: Synthesis of L- and D-α-Amino-Adipic Acids, L-α-aminopimelic Acid and Appropriate Unsaturated Derivatives. Tetrahedron Lett. 43: 4297-4308; and, Subasinghe et al., (1992) Quisqualic acid analogues: synthesis of beta-heterocyclic 2-aminopropanoic acid derivatives and their activity at a novel quisqualate-sensitized site. J. Med. Chem. 35: 4602-7. See also, US 2004/0198637 and US 2005/0170404, each of which is incorporated by reference herein in their entirety.

The terms “amino acid modification(s)” and “modification(s)” refer to amino acid substitutions, deletions or insertions or any combinations thereof in an amino acid sequence relative to another amino acid sequence, for example a native amino acid sequence. Substitutional variants herein are those that have at least one amino acid residue in a native CTLD sequence removed and a different amino acid inserted in its place at the same position. The substitutions may be single, where only one amino acid in the molecule has been substituted, or they may be multiple, where two or more amino acids have been substituted in the same molecule. Specific reference to more than one amino acid substitution in a CTLD refers to multiple substitutions in which each individual amino acid substitution can occur at any amino acid position within the CTLD, including consecutive and non-consecutive amino acid positions. Likewise, specific reference to more than one amino acid insertion or deletion in a CTLD refers to multiple insertions or deletions in which each individual amino acid insertion or deletion can occur at any amino acid position within the CTLD, including consecutive and non-consecutive amino acid positions.

The terms “nucleic acid molecule encoding”, “DNA sequence encoding”, and “DNA encoding” refer to the order or sequence of deoxyribonucleotides along a strand of deoxyribonucleic acid. The order of these deoxyribonucleotides determines the order of amino acids along the polypeptide chain. The DNA sequence thus encodes the amino acid sequence.

The terms “randomize,” “randomizing” and “randomized” as well as any similar terms used in any context to identify randomized polypeptide or nucleic acid sequences, refer to ensembles of polypeptide or nucleic acid sequences or segments, in which the amino acid residue or nucleotide at one or more sequence positions may differ between different members of the ensemble of polypeptides or nucleic acids, such that the amino acid residue or nucleotide occurring at each such sequence position may belong to a set of amino acid residues or nucleotides that may include all possible amino acid residues or nucleotides or any restricted subset thereof. The terms are often used to refer to ensembles in which the number of possible amino acid residues or nucleotides is the same for each member of the ensemble, but may also be used to refer to such ensembles in which the number of possible amino acid residues or nucleotides in each member of the ensemble may be any integer number within an appropriate range of integer numbers.

Turning now to the invention in more detail, in one aspect the invention is directed to a polypeptide having a multimerizing domain and at least one polypeptide binding member that binds to IL-23R. In accordance with the invention, the binding member may either be linked to the multimerizing domain, for example at the N- or the C-terminus. Also, in certain embodiments it may be advantageous to link a binding member, or two different binding members, that bind to IL-23R to both the N-terminus and the C-terminus of a multimerizing domain of the monomer, and thereby providing a multimeric polypeptide complex comprising six binding members capable of binding an IL-23R. In general, the polypeptides of the invention are non-natural polypeptides, for example, fusion proteins of a multimerizing domain and a polypeptide sequence that binds an IL-23R. The non-natural polypeptides may also be natural polypeptides wherein the naturally occurring amino acid sequence has been altered by the addition, deletion, or substitution of amino acids. Examples of such polypeptide include polypeptides having a C-type Lectin Like Domain (CTLD) wherein one or more of the loop regions of the domains have been modified as described herein. In other aspects of the invention, the polypeptide that binds to IL-23R is a fragment or variant of a natural polypeptide that binds to the receptor, wherein when the naturually occurring polypeptide, variant or fragment is fused to a multimerizing domain, the fusion protein is no longer a naturally occurring polypeptide. Accordingly, the invention does not exclude naturally occurring polypeptide, fragments or variants thereof from being a part of fusion protein of the invention.

In an embodiment of this aspect, the polypeptide is an IL-23R antagonist that binds to IL-23R and prevents signaling through the IL-23 pathway. In one embodiment, the polypeptide binds IL23-R (SEQ ID NO: 5) or variants thereof. The polypeptides of the invention bind to one or more sites on IL-23R that prevents binding of the native IL-23 ligand and thereby prevent activation of the receptor by the IL-23 ligand. Also, the polypeptides of the invention do not have agonist activity and do not activate the IL-23 heterdimeric receptor.

In a particular embodiment, the polypeptide does not specifically bind to IL-12Rβ1 or IL-12Rβ2. Accordingly, use of the polypeptide of the invention in therapeutic compositions can avoid the consequences of the unwanted blocking the activity of IL-12 for certain therapies.

In various aspects, a monomeric polypeptide of the invention includes at least two segments: a multimerizing domain that is capable of forming a multimeric complex with other multimerizing domains, and a polypeptide sequence that binds to IL-23R. The sequence that binds to IL-23R may be fused with the multimerizing domain at the N-terminus, at the C-terminus, or at both the N- and C-termini of the domain. In one embodiment, the polypeptide that binds to IL-23R at the N-terminus is different than the polypeptide that binds IL-23R at the C terminus of the trimerizing domain.

In one embodiment, a first polypeptide that binds IL-23R is fused at one of the N-terminus and the C-terminus of a trimerizing domain, and a second polypeptide that is a modulator of inflammation is fused at the other of the N-terminus or the C-terminus of the trimerizing domain. Modulators that are not polypeptides can be linked to the trimerizing domain, either covalently or non-covalently, as would be understood by one of skill in the art. In addition to modulators of inflammation, other polypeptide and non-polypeptide therapeutic agents can be linked to the trimerizing module.

For the treatment of cancer, it could be desirable to target the polypeptides of the invention to the tumor environment to more effectively prevent the tumor-promoting action of IL-23 on tumor cells. Therefore, another aspect of the invention includes a multimerizing domain having a polypeptide that binds to IL-23R on one end of the domain (one of either of the N-terminus or C-terminus), and a polypeptide that binds to tumor-associated (TAA) or tumor-specific antigens (TSA) on the other end (the other of the N-terminus and the C-terminus). The domain that binds to TAA's or TSA's may be peptides, such as for example CTLDs, single chain antibodies, or any type of domain that specifically binds to the desired target.

In one particular approach the activity of death receptor agonists can be enhanced by designing a molecule with binding activity mediated through an IL-23R binding polypeptide one end of a trimerizing domain that drives the drug to sites of inflammation in the setting of cancer and that allows clustering of the death receptor specific polypeptide on the second end of the trimerizing domain. In various aspects, the polypeptide binds to a death receptors at lower affinity than to IL-23R. More specifically, the polypeptide that binds to IL-23R may bind with least 2 times greater affinity, for example, 2, 2.5, 3, 3.5, 4, 4.5 5, 10, 15, 20, 50 and 100 times greater, than the polypeptide binds the death receptor.

Indications for trimeric complexes having both IL-23R-binding polypeptide(s) and TAA or TSA targeting agent(s) include non-small cell lung cancer (NSCLC), colorectal cancer, ovarian cancer, renal cancer, pancreatic cancer, sarcomas, non-hodgkins lymphoma (NHL), multiple myeloma, breast cancer, prostate cancer, melanoma, glioblastoma, neuroblastoma.

In another aspect, a polypeptide that specifically binds to an IL-23 receptor is contained in the loop region of a CTLD. The polypeptide may be a portion of the IL-23 polypeptide, or may be sequence that is identified as provided here. In this aspect the sequence is contained in a loop region of a CLTD, and the CTLD is fused to a trimerizing domain at the N-terminus or C-terminus of the domain either directly or through the appropriate linker. Also, the polypeptide of the invention may include a second CLTD domain, fused at the other of the N-terminus and C-terminus, wherein the sequence of the CTLDs and/or their affinity for IL-23R may be the same or different. In a variation of this aspect, the polypeptide includes a polypeptide that binds to an IL-23R at one of the termini of the trimerizing domain and a CLTD at the other of the termini. One, two or three of the polypeptides can be part of a trimeric complex containing up to six specific binding members for IL-23R.

The polypeptide sequences that bind IL-23R can have a binding affinity for IL-23R that is about equal to the binding affinity that native IL-23 has for IL-23R. In certain embodiments, the polypeptides of the invention have a binding affinity for the IL-23R that is greater or less than the binding affinity that native IL-23 has for the same IL-23R.

The polypeptides of the invention can include one or more amino acid mutations in a native IL-23 (p19) sequence, or a random sequence, that has selective binding affinity for IL-23R, but not IL-12Rβ1 or IL-12Rβ2. For example, when binding affinity of such binding members to the IL-23R is approximately equal (unchanged) or greater than (increased) as compared to native IL-23, and the binding affinity of the binding member to IL-12Rβ1 or IL-12Rβ2 is less than or nearly eliminated as compared to native sequence IL-23, the binding affinity of the binding member, for purposes herein, is considered “selective” for IL-23R. In another example, the affinity of the binding member for IL-23R is less than the affinity of IL-23 for the receptor, but the binding member is still selective for the receptor if it has greater affinity for IL-23R than its affinity for IL-12Rβ1 or IL-12Rβ2. Preferred IL-23R selective antagonists of the invention will have at least 5-fold, preferably at least a 10-fold greater binding affinity to IL-23R as compared to IL-12Rβ1 or IL-12Rβ2, and even more preferably, will have at least 100-fold greater binding affinity to IL-23R as compared to a IL-12Rβ1 or IL-12Rβ2.

The respective binding affinity of the antagonists can be determined and compared to the binding properties of native IL-23, or a portion thereof, by ELISA, RIA, and/or BIAcore assays, known in the art. Preferred IL-23R selective antagonists of the invention will not inhibit IL-12 signaling in at least one type of mammalian cell, and such signal inhibition can be determined by known art methods such as ELISA.

In an embodiment, IL-23R antagonist comprises an antibody or an antibody fragment. In the present context, the term “antibody” is used to describe an immunoglobulin whether natural or partly or wholly synthetically produced. As antibodies can be modified in a number of ways, the term “antibody” should be construed as covering any specific binding member or substance having a binding domain with the required receptor specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. The term also covers any polypeptide or protein having a binding domain which is, or is homologous to, an antibody binding domain, e.g. antibody mimics. These can be derived from natural sources, or they may be partly or wholly synthetically produced. Examples of antibodies are the immunoglobulin isotypes and their isotypic subclasses; fragments which comprise an antigen binding domain such as Fab, Fab′, F(ab′)₂, scFv, Fv, dAb, Fd; and diabodies.

In another aspect the invention relates to a multimeric complex of three polypeptides, each of the polypeptides comprising a multimerizing domain and at least one polypeptide that binds to IL-23R. In an embodiment, the multimeric complex comprises a polypeptide having a multimerizing domain selected from a polypeptide having substantial homology to a human tetranectin trimerizing structural element, or other human trimerizing polyeptides including mannose binding protein (MBP) trimerizing domain, a collectin neck region polypeptide, and others. The multimeric complex can be comprised of any of the polypeptides of the invention wherein the polypeptides of the multimeric complex comprise multimerizing domains that are able to associate with each other to form a multimer. Accordingly, in some embodiments, the multimeric complex is a homomultimeric complex comprised of polypeptides having the same amino acid sequences. In other embodiments, the multimeric complex is a heteromultimeric complex comprised of polypeptides having different amino acid sequences such as, for example, different multimerizing domains, and/or different polypeptides that bind to an IL-23R. In addition the heteromultimeric complexes can include a therapeutic agent and IL-23R antagonists.

Further, in one aspect, the invention relates to a method for preparing a polypeptide that prevents activation of IL-23R in a cell expressing IL-23R. The method includes the steps of: (a) selecting a first polypeptide(s) that specifically binds IL-23R; (b) grafting the first polypeptide(s) into one or two loop regions of tetranectin CTLD to form a first binding determinant or directly fusing the polypeptide to the tetranectin trimerizing domain, and (c) fusing the first CTLD with one of the N-terminus or the C-terminus of a tetranectin trimerizing domain. In one particular embodiment of the method, the polypeptide that binds IL-23R does not bind IL-12Rβ1 or IL-12Rβ2.

The tetranectin CTLD has up to five loop regions into which binding members for IL-23R may be inserted or identified by selection from a randomized library as described here. Accordingly, when a polypeptide of the invention includes a CTLD, the polypeptide may have up to five binding members for IL-23R attached to the trimerizing domain through the CTLD. Each of the binding members may be the same or different.

In other aspects of the polypeptides of the invention, a receptor antagonist can be bound to one terminus of a trimerizing domain and one or more therapeutic agents may be bound to the second terminus. The agent may be bound directly or through an appropriate linker as understood to those of skill in the art. Such agents may act in the same pathway as the antagonist, or may act in a different pathway for immune disorders, cancers and other conditions. In addition to being bound to one of the termini of the polypeptides, the agent may be covalently linked to the trimerizing domain via a peptide bond to a side chain in the trimerizing domain or via a bond to a cysteine residue. Other ways of covalently coupling the agent to the module can also be used as shown in, for example, U.S. Pat. No. 6,190,886, which is incorporated by reference herein.

Identification of Polypeptide Sequences Specific for IL-23R

In one aspect, a specific binding member for IL-23R can be obtained from a random library of polypeptides by selection of members of the library that specifically bind to the receptor. A number of systems for displaying phenotypes with putative ligand binding sites are known. These include: phage display (e.g. the filamentous phage fd [Dunn (1996), Griffiths and Duncan (1998), Marks et al. (1992)], phage lambda [Mikawa et al. (1996)]), display on eukaryotic virus (e.g. baculovirus [Ernst et al. (2000)]), cell display (e.g. display on bacterial cells [Benhar et al. (2000)], yeast cells [Boder and Wittrup (1997)], and mammalian cells [Whitehorn et al. (1995)], ribosome linked display [Schaffitzel et al. (1999)], and plasmid linked display [Gates et al. (1996)].

Also, US2007/0275393, which is incorporated herein by reference in its entirety, specifically describes a procedure for accomplishing a display system for the generation of CLTD libraries. The general procedure includes (1) identification of the location of the loop-region, by referring to the 3D structure of the CTLD of choice, if such information is available, or, if not, identification of the sequence locations of the β2, β3 and β4 strands by sequence alignment with known sequences, as aided by the further corroboration by identification of sequence elements corresponding to the β2 and β3 consensus sequence elements and β4-strand characteristics, also disclosed above; (2) subcloning of a nucleic acid fragment encoding the CTLD of choice in a protein display vector system with or without prior insertion of endonuclease restriction sites close to the sequences encoding β2, β3 and β4; and (3) substituting the nucleic acid fragment encoding some or all of the loop-region of the CTLD of choice with randomly selected members of an ensemble consisting of a multitude of nucleic acid fragments which after insertion into the nucleic acid context encoding the receiving framework will substitute the nucleic acid fragment encoding the original loop-region polypeptide fragments with randomly selected nucleic acid fragments. Each of the cloned nucleic acid fragments, encoding a new polypeptide replacing an original loop-segment or the entire loop-region, will be decoded in the reading frame determined within its new sequence context.

A complex may be formed that functions as a homo-trimeric protein that blocks natural IL-23 from binding and activating IL-23R. However peptides with IL-23R binding activity must be identified first. To accomplish this, peptides with known binding activity can be used or additional new peptides identified by screening from display libraries. A number of different display systems are available, such as but not limited to phage, ribosome and yeast display.

To select for new peptides with binding activity, libraries can be constructed and initially screened for binding to IL-23R, either as single monomeric CTLD domains, or individual peptides displayed on the surface of phage. Once sequences with IL-23R binding activity have been identified these sequences would subsequently be grafted on to the trimerization domain of human tetranectin to create potential protein therapeutics capable of binding IL-23R.

Four main strategies may be employed in the construction of these phage display libraries and trimerization domain constructs. The first strategy would be to construct and/or use random peptide phage display libraries. Random linear peptides and/or random peptides constructed as disulfide constrained loops would be individually displayed on the surface of phage particles and selected for binding to the desired IL-23R through phage display “panning”. After obtaining peptide clones with IL-23R binding activity, these peptides would be grafted on to the trimerization domain of human tetranectin or into loops of the CTLD domain followed by grafting on the trimerization domain and screened for antagonist activity.

A second strategy for construction of phage display libraries and trimerization domain constructs would include obtaining CTLD derived binders. Libraries can be constructed by randomizing the amino acids in one or more of the five different loops within the CTLD scaffold of human tetranectin displayed on the surface of phage. Binding to the IL-23R can be selected for through phage display panning. After obtaining CTLD clones with peptide loops demonstrating IL-23R binding activity, these CTLD clones can then be grafted on to the trimerization domain of human tetranectin and screened for antagonist activity.

A third strategy for construction of phage display libraries and trimerization domain constructs would include taking known sequences with binding capabilities to IL-23R and graft these directly on to the trimerization domain of human tetranectin and screen for binding activity.

A fourth strategy includes using peptide sequences with known binding capabilities to the IL-23R and first improve their binding by creating new libraries with randomized amino acids flanking the peptide or/and randomized selected internal amino acids within the peptide, followed by selection for improved binding through phage display. After obtaining binders with improved affinity, the binders of these peptides can be grafted on to the trimerization domain of human tetranectin and screening for antagonist activity. In this method, initial libraries can be constructed as either free peptides displayed on the surface of phage particles, as in the first strategy (above), or as constrained loops within the CTLD scaffold as in the second strategy also discussed above. After obtaining binders with improved affinity, grafting of these peptides on to the trimerization domain of human tetranectin and screening for antagonist activity would occur.

Versions of the trimerization domain can be used that either eliminate up to 16 residues at the N-terminus (V17), or alter the C-terminus. C-terminal variations termed Trip V [SEQ ID NO: 60], TripT [SEQ ID NO: 61], TripQ [SEQ ID NO: 62] and TripK [SEQ ID NO: 59] See FIG. 2) allow for unique presentation of the CTLD domains on the trimerization domain. TripV, TripT, TripQ represent fusions of the CTLD molecule directly onto the trimerization module without any structural flexibility but are turning the CTLD molecule ⅓^{rd} going from TripV to TripT and from TripT to TripQ. This is due to the fact that each of these amino acids is in an α-helical turn and 3.2 aa are needed for a full turn. Free peptides selected for binding in the first, third and fourth strategies can be grafted onto any of above versions of the trimerization domain. Resulting fusions can then be screened to see which combination of peptide and orientation gives the best activity. Peptides selected for binding constrained within the loops of the CTLD of tetranectin can be grafted on to the full length trimerization domain.

More particularly, the four strategies are described below. Although these strategies focus on phage display, other equivalent methods of identifying polypeptides can be used.

Strategy 1

Peptide display library kits such as, but not limited to, the New England Biolabs Ph.D. Phage display Peptide Library Kits are sold commercially and can be purchased for use in selection of new and novel peptides with IL-23R binding activity. Three forms of the New England Biolabs kit are available: the Ph.D.-7 Peptide Library Kit containing linear random peptides 7 amino acids in length, with a library size of 2.8×10⁹ independent clones, the Ph.D.-C7C Disulfide Constrained Peptide Library Kit containing peptides constructed as disulfide constrained loops with random peptides 7 amino acids in length and a library size of 1.2×10⁹ independent clones, and the Ph.D.-12 Peptide Library Kit containing linear random peptides 12 amino acids in length, with a library size of 2.8×10⁹ independent clones.

Alternatively similar libraries can be constructed de novo with peptides containing random amino acids similar to these kits. For construction random nucleotides are generated using either an NNK, or NNS strategy, in which N represents an equal mixture of the four nucleic acid bases A, C, G and T. The K represents an equal mixture of either G or T, and S represents and equal mixture of either G or C. These randomized positions can be cloned onto to the Gene III protein in either a phage or phagemid display vector system. Both the NNK and the NNS strategy cover all 20 possible amino acids and one stop codon with slightly different frequencies for the encoded amino acids. Because of the limitations of bacterial transformation efficiency, library sizes generated for phage display are in the order of those started above, thus peptides containing up to 7 randomized amino acids positions can be generated and yet cover the entire repertoire of theoretical combinations (20⁷=1.28×10⁹). Longer peptide libraries can be constructed using either the NNK or NNS strategy however the actual phage display library size likely will not cover all the theoretical amino acid combinations possible associated with such lengths due to the requirement for bacterial transformation.

Thus ribosome display libraries might be beneficial where larger/longer random peptides are involved. For disulfide constrained libraries a similar NNK or NNS random nucleotide strategy is used. However, these random positions are flanked by cysteine amino acid residues, to allow for disulfide bridge formation. The N terminal cysteine is often preceded by an additional amino acid such as alanine. In addition a flexible linker made up to but not limited to several glycine residues may act as a spacer between the peptides and the gene III protein for any of the above random peptide libraries.

Strategy 2

The human tetranectin CTLD shown in FIGS. 4 and 5 contains five loops (four loops in LSA and one loop comprising LSB), which can be altered to confer binding of the CTLD to different protein targets. Random amino acid sequences can be placed in one or more of these loops to create libraries from which CTLD domains with the desired binding properties can be selected. Construction of these libraries containing random peptides constrained within any or all of the five loops of the human tetranectin CTLD can be accomplished (but is not limited to) using either a NNK or NNS as described above in strategy 1. A single example of a method by which seven random peptides can be inserted into loop 1 of the TN CTLD is as follows.

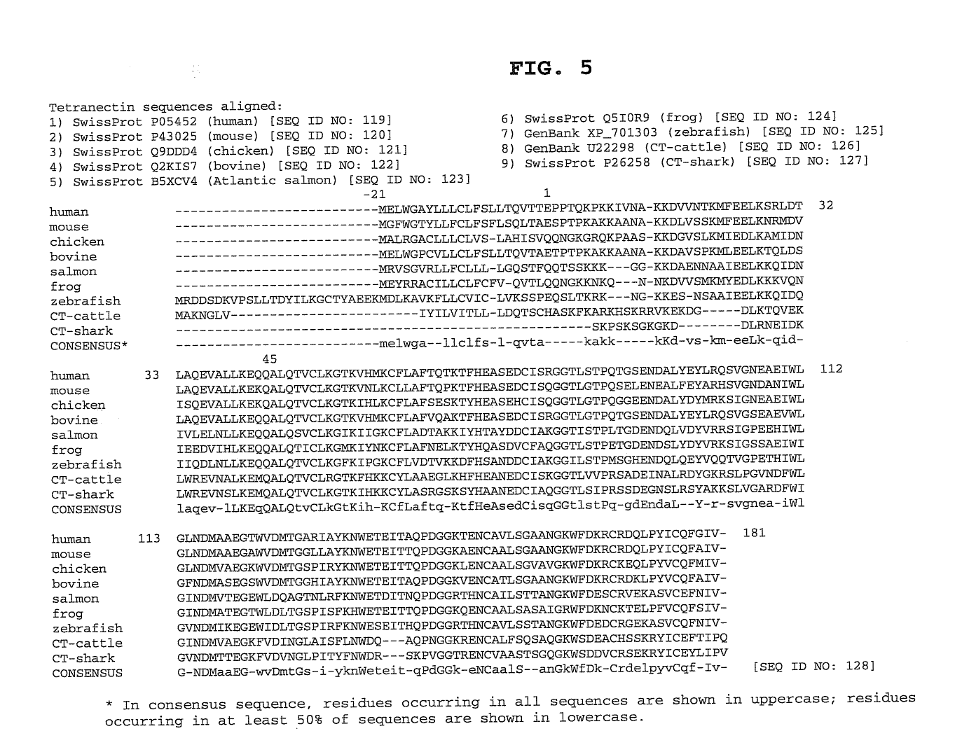

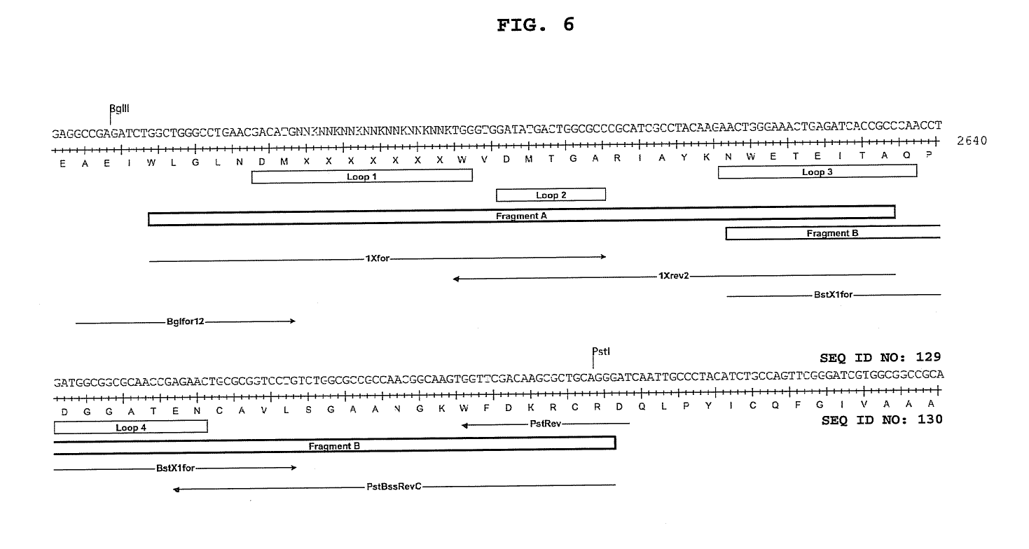

PCR can be accomplished using primers 1X for (SEQ ID NO: 224) and 1X rev2 (SEQ ID NO: 226) in a PCR reaction without template to generate fragment A, and primers BstX1 for (SEQ ID NO: 227) and PstBssRevC (SEQ ID NO: 228) can be used in a separate PCR reaction without template to generate fragment B. PCR can be performed using a high fidelity polymerase or taq blend and standard PCR thermocycling conditions. These two overlapping fragments can then be purified and used together, along with the outer primers Bglfor12 (SEQ ID NO: 229) and PstRev (SEQ ID NO: 230), to generate the desired DNA fragment by PCR. Digestion with the restriction enzymes Bgl II and PstI, or other appropriate restriction enzymes when using other primers, permits gel isolation of the fragment containing the loops or some portion thereof of the TN CTLD. This purified fragment can then be ligated into a similarly digested phage display vector such as pPHCPAB (SEQ ID NO:150) or pANA27 (SEQ ID NO: 164) containing the restriction modified CTLD fused to Gene III, (See FIG. 6).

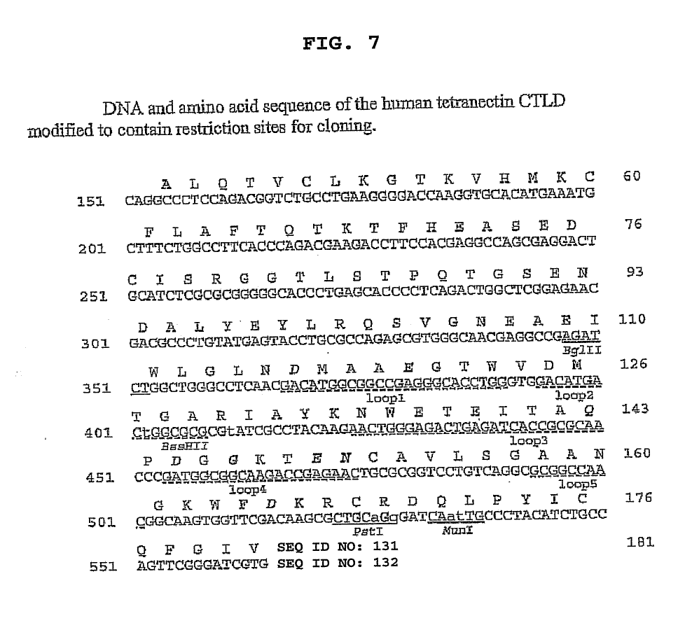

Modification of other loops by replacement with randomized amino acids can be similarly performed as shown above. The replacement of defined amino acids within a loop with randomized amino acids is not restricted to any specific loop, nor is it restricted to the original size of the loops. Likewise, total replacement of the loop is not required, partial replacement is possible for any of the loops. In some cases retention of some of the original amino acids within the loop, such as the calcium coordinating amino acids shown in FIG. 7 may be desirable. In these cases, replacement with randomized amino acids may occur for either fewer of the amino acids within the loop to retain the calcium coordinating amino acids, or additional randomized amino acids may be added to the loop to increase the overall size of the loop yet still retain these calcium coordinating amino acids. Very large peptides can be accommodated and tested by combining loop regions such as loops 1 and 2 or loops 3 and 4 into one larger replacement loop. In addition, other CTLDs, such as but not limited to the MBL CTLD, can be used instead of the CTLD of tetranectin. Grafting of peptides into these CTLDs can occur using methods similar to those described above.

In various exemplary aspects of the invention, the polypeptides that bind to an IL-23R can be identified using a combinatorial peptide library, and a library of nucleic acid sequences encoding the polypeptides of the library, based upon a CTLD backbone, wherein the CTLDs of the polypeptides have been modified according to a number of exemplary schemes, which have been labeled for the purposes of identification only as Schemes (a)-(h):

In one aspect, the invention provides a combinatorial peptide library, and a library of nucleic acid sequences encoding the polypeptides of the library, wherein the CTLDs of the polypeptides have been modified according to a number of schemes, which have been labeled for the purposes of identification only as Schemes (a)-(j). While each scheme is more particularly described herein, the modifications are at least as follows:

(a) amino acid modifications in at least one of four loops in loop segment A (LSA) of the CTLD, wherein the amino acid modifications comprise an insertion of at least one amino acid in Loop 1 and random substitution of at least five amino acids within Loop 1;

(b) amino acid modifications in at least one of four loops in loop segment A (LSA) of the CTLD, wherein the amino acid modifications comprise random substitution of at least five amino acids within Loop 1 and random substitution of at least three amino acids within Loop 2;

(c) amino acid modifications in at least one of four loops in loop segment A (LSA) of the CTLD, wherein the amino acid modifications comprise random substitution of at least seven amino acids within Loop 1 and at least one amino acid insertion in Loop 4;

(d) amino acid modifications in at least one of four loops in loop segment A (LSA) of the CTLD, wherein the amino acid modifications comprise at least one amino acid insertion in Loop 3 and random substitution of at least three amino acids within Loop 3;

(e) amino acid modifications in at least one of four loops in loop segment A (LSA) of the CTLD, wherein the amino acid modifications comprise a modification that combines two loops into a single loop, wherein the two combined loops are Loop 3 and Loop 4;

(f) amino acid modifications in at least one of four loops in loop segment A (LSA) of the CTLD, wherein the amino acid modifications comprise at least one amino acid insertion in Loop 4 and random substitution of at least three amino acids within Loop 4;

(g) amino acid modifications in at least one of the five loops in loop segment A (LSA) and loop segment B (LSB) of the CTLD, wherein the amino acid modifications comprise random substitution of at least five amino acid residues in Loop 3 and random substitution of at least three amino acids within Loop 5;

(h) amino acid modifications in at least one of the four loops in loop segment A (LSA) of the CTLD, wherein the amino acid modifications comprise random substitution of at least one amino acid and insertion of at least six amino acids in Loop 3;

(i) amino acid modifications in at least one of the four loops in the loop segment A (LSA) of the CTLD, wherein the amino acid modifications comprise a mixture of (1) random substitution of at least six amino acids in Loop 3 and (2) random substitution of at least six amino acids and at least one amino acid insertion in Loop 3; and

(j) amino acid modifications in at least one of the four loops in the loop segment A (LSA) of the CTLD, wherein the amino acid modifications comprise at least four or more amino acid insertions in at least one of the four loops in the loop segment A (LSA) or loop 5 in loop segment B (LSB) of the CTLD.

With respect to scheme (a), the invention provides a combinatorial polypeptide library comprising polypeptide members having a randomized C-type lectin domain (CTLD), wherein the randomized CTLD includes amino acid modifications in at least one of the four loops in LSA or in the loop in LSB of the CTLD, wherein the amino acid modifications comprise at least one amino acid insertion in Loop 1 and random substitution of at least five amino acids within Loop 1.

In certain embodiments of this aspect of the combinatorial library, when the CTLD is from human tetranectin, the CTLD also has a random substitution of Arginine-130. For CTLDs other than the CTLD of human tetranectin, this peptide is located immediately adjacent to the C-terminal peptide of Loop 2 in the C-terminal direction. For example, in mouse tetranectin, this peptide is Gly-130. In certain embodiments of this aspect of the combinatorial library, when the CTLD is from human or mouse tetranectin, the CTLD includes a substitution of Lysine-148 to Alanine in Loop 4.

In certain embodiments, when the combinatorial library has the modified CTLD of Scheme (a), the amino acid modifications comprise two amino acid insertions in Loop 1 and random substitution of at least five amino acids within Loop 1. In other embodiments, when the combinatorial library has the modified CTLD of scheme (a) and the CTLD is from human tetranectin, the amino acid modifications comprise at least one amino acid insertion in Loop 1, random substitution of at least five amino acids within Loop 1, and include a random substitution of Arginine 130. In one specific embodiment, when the combinatorial library has the modified CTLD of scheme (a) and the CTLD is from human tetranectin, the amino acid modifications comprise two amino acid insertions in Loop 1, random substitution of five amino acids within Loop 1, and a random substitution of Arginine 130. In one specific embodiment, when the combinatorial library has the modified CTLD of scheme (a) and the CTLD is from mouse tetranectin, the amino acid modifications comprise two amino acid insertions in Loop 1, random substitution of five amino acids within Loop 1, and a random substitution of Leucine 130. In any of the embodiments for scheme (a), the amino acid modifications can further comprise a substitution of Lysine-148 to Alanine Thus, in one specific embodiment of this aspect of the combinatorial library, the CTLD comprises two amino acid insertions in Loop 1, random substitution of at least five amino acids within Loop 1, random substitution of Arginine-130 or other amino acid located outside and adjacent to loop 2 in the C-terminal direction, and a substitution of lysine-148 to alanine in Loop 4.

With respect to scheme (b), the invention provides a combinatorial polypeptide library comprising polypeptide members having a randomized C-type lectin domain (CTLD), wherein the randomized CTLD comprises amino acid modifications in at least one of the four loops in the LSA of the CTLD, wherein the amino acid modifications comprise random substitution of at least five amino acids within Loop 1 and random substitution of at least three amino acids within Loop 2.

In certain embodiments of this aspect of the combinatorial library of scheme (b), when the CTLD is from tetranectin, the amino acid modifications comprise random substitution of at least five amino acids within Loop 1, random substitution of at least three amino acids within Loop 2, and random substitution of Arginine-130, or other amino acid located outside and adjacent to loop 2 in the C-terminal direction. In certain embodiments, when the combinatorial library has the modified CTLD of Scheme (b) and the CTLD is from human tetranectin, the amino acid modifications include random substitutions of at least five amino acids in Loop 1, random substitution of at least three amino acids in Loop 2, and include a random substitution of Arginine 130. In one embodiment, when the combinatorial library has the modified CTLD of Scheme (b) and the CTLD is from human tetranectin, the amino acid modifications include random substitutions of five amino acids in Loop 1, random substitution of three amino acids in Loop 2, and a random substitution of Arginine 130. In certain other embodiments, when the combinatorial library has the modified CTLD of Scheme (b) and the CTLD is from mouse tetranectin, the amino acid modifications include random substitutions of at least five amino acids in Loop 1, random substitution of at least three amino acids in Loop 2, and include a random substitution of Leucine 130. In one embodiment, when the combinatorial library has the modified CTLD of Scheme (b) and the CTLD is from mouse tetranectin, the amino acid modifications include random substitutions of five amino acids in Loop 1, random substitution of three amino acids in Loop 2, and a random substitution of Leucine 130. In any of the embodiments for scheme (b), the amino acid modifications can further comprise a substitution of Lysine-148 to Alanine. Thus, in one specific embodiment, the amino acid modifications comprise random substitution of at least five amino acids within Loop 1, random substitution of at least three amino acids within Loop 2, and random substitution of Arginine-130, or other amino acid located outside and adjacent to loop 2 in the C-terminal direction and a substitution of Lysine-148 to Alanine in Loop 4.

With respect to scheme (c), the invention provides a combinatorial polypeptide library comprising polypeptide members that have a randomized C-type lectin domain (CTLD), wherein the randomized CTLD comprises amino acid modifications in at least one of the four loops in loop segment A (LSA) of the CTLD, wherein the amino acid modifications comprise random substitution of at least seven amino acids within Loop 1 and at least one amino acid insertion in Loop 4.

In certain embodiments of this aspect of the combinatorial library, the polypeptide members of the combinatorial library further comprise random substitution of at least two amino acids within Loop 4. In certain other embodiments of this aspect, the amino acid modifications comprise three amino acid insertions within Loop 4 and optionally further comprise random substitution of at least two amino acids. In one embodiment, the amino acid modifications comprise random substitution of at least seven amino acids within Loop 1, at least three amino acid insertions in Loop 4, and random substitution of at least two amino acids within Loop 4. In one specific embodiment, the amino acid modifications comprise random substitution of seven amino acids within Loop 1, three amino acid insertions in Loop 4, and random substitution of two amino acids within Loop 4.

With respect to scheme (d), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise at least one amino acid insertion in loop 3 and random substitution of at least three amino acids within Loop 3.

In certain embodiments, when the combinatorial library has the modified CTLD of Scheme (d), the amino acid modifications can further comprise at least one amino acid insertion in Loop 4, and can further comprise random substitution of at least three amino acids within Loop 4. In any of the described embodiments for scheme (d), the amino acid modifications can comprise three amino acid insertions in Loop 3. In any of the described embodiments for scheme (d), the amino acid modifications can comprise three amino acid insertions in Loop 4. Thus, in certain embodiments, the amino acid modifications comprise random substitution of at least three amino acids within Loop 3, random substitution of at least three amino acids within Loop 4, at least one amino acid insertion in Loop 3 and at least one amino acid insertion in Loop 4. In certain embodiments, the amino acid modifications comprise random substitution of at least three amino acids within Loop 3, random substitution of at least three amino acids within Loop 4, at least three amino acid insertions in Loop 3 and at least three amino acid insertions in Loop 4. In one specific embodiment, the amino acid modifications comprise random substitution of three amino acids within Loop 3, random substitution of three amino acids within Loop 4, three amino acid insertions in Loop 3, and three amino acid insertions in Loop 4. In any of the described embodiments, when the CTLD is tetranectin, the amino acid modifications can further compr random substitution of Lysine-148 to Alanine or in Loop 4.

With respect to scheme (e), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise a modification that combines two Loops into a single Loop, wherein the two combined Loops are Loop 3 and Loop 4. In certain embodiments, when the members of the combinatorial library have the modified CTLD of Scheme (e), the amino acid modifications comprise random substitution of at least six amino acids within Loop 3 and random substitution of at least four amino acids within Loop 4. In one specific embodiment, the amino acid modifications comprise random substitution of six amino acids within Loop 3 and random substitution of four amino acids within Loop 4. In any of the embodiments for scheme (e), when the CTLD is from human tetranectin, the amino acid modifications can further comprise random substitution of Proline-144. In one specific embodiment, when the CTLD is from human tetranectin, the amino acid modifications comprise random substitution of six amino acids within Loop 3, random substitution of four amino acids within Loop 4, and a random substitution of proline 144, resulting in a combined Loop 3 and Loop 4 amino acid sequence, comprising, for example, NWEXXXXXXX XGGXXXN (SEQ ID NO: 468), wherein X is any amino acid and wherein the amino acid sequence of SEQ ID NO: 468 forms a single Loop region. Thus, in one specific embodiment, the polypeptide members of the combinatorial library comprise the sequence NWEXXXXXXX XGGXXXN (SEQ ID NO: 468), wherein X is any amino acid and wherein the amino acid sequence of SEQ ID NO: 468 forms a single loop from combined and modified Loop 3 and Loop 4.

With respect to scheme (f), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise at least one amino acid insertion in Loop 4 and random substitution of at least three amino acids within Loop 4. In certain embodiments, the amino acid modifications comprise four amino acid insertions in Loop 4. In one embodiment, the amino acid modifications comprise at least four amino acid insertions in Loop 4 and random substitution of at least three amino acids within Loop 4. In one specific embodiment, the amino acid substitutions comprise four amino acid insertions in Loop 4 and random substitution of three amino acids within Loop 4.

With respect to scheme (g), the polypeptide members of the combinatorial library comprise a modified Loop 3 and a modified Loop 5, wherein the modified Loop 3 comprises randomization of five amino acid residues and the modified Loop 5 comprises randomization of three amino acid residues. In one embodiment, the polypeptide members of the combinatorial library comprise a modified Loop 3, a modified Loop 5, and a modified Loop 4, wherein the modification to Loop 4 abrogates plasminogen binding. For example, when the combinatorial library has the modified CTLD of Scheme (g), and the CTLD is from human tetranectin, the amino acid modifications can further comprise one or more amino acid modifications in Loop 4 that modulates plasminogen binding affinity of the CTLD, for example, the substitution of Lysine 148 to Alanine Thus, in certain embodiments, when the CTLD is from human tetranectin, the amino acid modifications comprise random substitution of at least five amino acid residues in Loop 3, random substitution of at least three amino acid residues in Loop 5, and substitution of Lysine 148 to Alanine in Loop 4. In one specific embodiment, the amino acid modifications comprises random substitution of five amino acid residues in Loop 3 and random substitution of three amino acid residues in Loop 5, and, in another specific embodiment, when the CTLD is from human tetranectin, the amino acid modifications further comprise substitution of Lysine 148 to Alanine in Loop 4.

With respect to scheme (h), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise random substitution of at least one amino acid and at least six amino acid insertions. In certain embodiments, when the CTLD is from human tetranectin, the amino acid modifications can further comprise one or more amino acid modifications in Loop 4 that modulates plasminogen binding affinity of the CTLD, for example, the substitution of lysine 148 to Alanine. In certain embodiments when the CTLD is from human tertranectin, the members of the combinatorial library have random substitution of at least one amino acid and insertion of at least six amino acids in Loop 3, and substitution of Lysine 148 to Alanine in Loop 4. In one specific embodiment, the amino acid modifications comprise random substitution of one amino acid and insertion of six amino acids in Loop 3. In one specific embodiment, when the CTLD is from human tertranectin, the members of the combinatorial library have random substitution of one amino acid and insertion of six amino acids in Loop 3, and substitution of lysine 148 to alanine in Loop 4. In any of the these embodiments when the CTLD is from human tetranectin, one of the substitutions is the substitution of Isoleucine 140.

With respect to scheme (i), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise a mixture of random substitution of six amino acids in Loop 3 and random substitution of six amino acids and one amino acid insertion in Loop 3. In one embodiment, the mixture further comprises random substitution of six amino acids and two amino acid insertions in Loop 3. Thus in one embodiment, the amino acid modifications comprises a mixture of random substitution of six amino acids in Loop 3, random substitution of six amino acids and one amino acid insertion in Loop 3, and random substitution of six amino acids and two amino acid insertions in Loop 3. In any of the embodiments of scheme (i), when the CTLD is from human tetranectin, the amino acid modifications further comprise a substitution of Lysine 148 to Alanine in Loop 4.

With respect to scheme (i), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications in at least one of the four loops in the loop segment A (LSA) of the CTLD, wherein the amino acid modifications comprise at least four or more amino acid insertions in at least one of the four loops in the loop segment A (LSA) or loop 5 in loop segment B (LSB) of the CTLD.

In embodiments wherein the combinatorial library comprises one or more amino acid modifications to the Loop 4 region (alone or in combination with modifications to other regions of the CTLD), certain of the modification(s) are designed to maintain, modulate, or abrogate the metal ion-binding affinity of the CTLD. Such modifications affect the plasminogen-binding activity of the CTLD (see, e.g., Nielbo, et al., Biochemistry, 2004, 43 (27), pp 8636-8643; or Graversen 1998).

The polypeptide members of the libraries can comprise one or more amino acid modifications (e.g., by insertion, substitution, extension, or randomization) in any combination of the four LSA loops and the LSB loop (Loop 5) of the CTLD. Thus, in any of the various embodiments described herein, the randomized CTLD can comprise one or more amino acid modifications in the loop of the LSB loop region (Loop 5), either alone, or in combination with one or more amino acid modifications in any one, two, three, or four loops of the LSA loop region (Loops 1-4). In one aspect, the invention provides a combinatorial polypeptide library comprising polypeptide members that have a randomized C-type lectin domain (CTLD), wherein the randomized CTLD comprises one or more amino acid modifications in at least one of the four loops in loop segment A (LSA) and one or more amino acid modifications in the loop in loop segment B (LSB) (Loop 5) of the CTLD, wherein the one or more amino acid modifications comprises randomization of the LSB amino acid residues.

According to the various embodiments described herein, the polypeptide members of the combinatorial libraries can have one or more amino acid modifications in any two, three, four, or five loops in the loop region (LSA and LSB) of the CTLD (e.g., any random combination of random amino acid modifications to two loops, to three loops, to four loops, or to all five loops). The polypeptide members of the combinatorial libraries can further comprise additional amino acid modifications to regions of the CTLD outside of the loop region (LSA and LSB), such as in the α-helices or β-strands (see, e.g., FIG. 1).

In further embodiments of the invention, the CTLD loop regions can be extended beyond the exemplary constructs detailed in the non-limiting Examples below.

In one aspect, the invention also provides a library of nucleic acid molecules encoding polypeptides of the combinatorial polypeptide library according to any one of the above-described aspects and embodiments. In one embodiment of this aspect, the invention provides a library of nucleic acid sequences encoding the polypeptides of the library, wherein the CTLDs of the polypeptides have been modified according to Schemes (a)-(j).

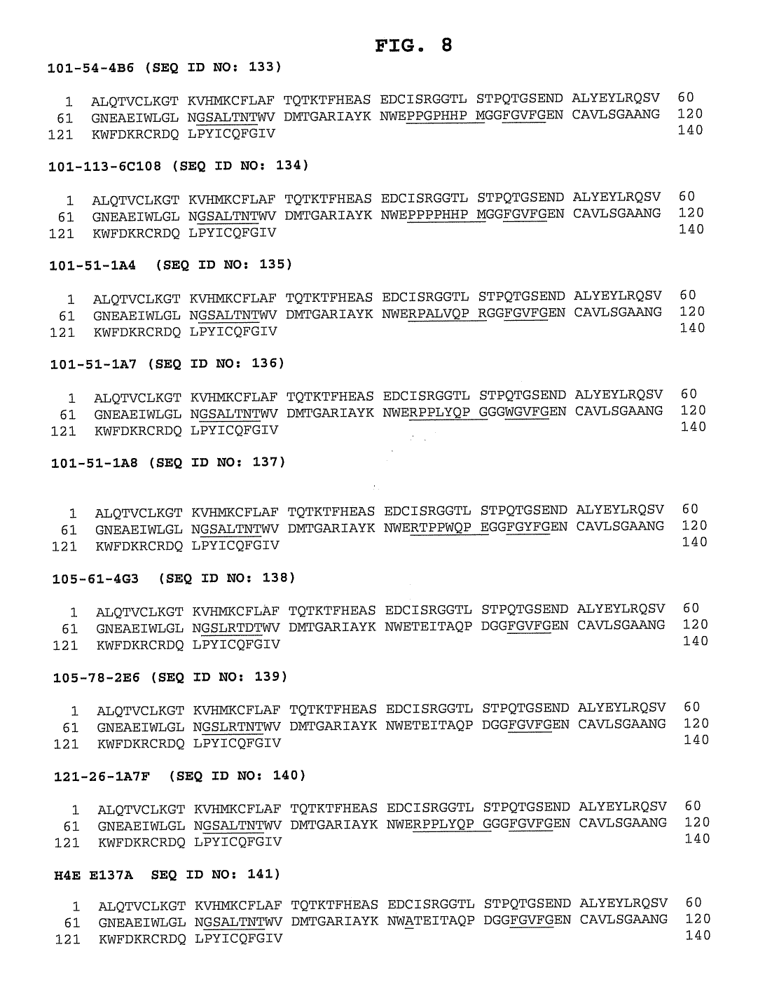

As more fully described in the Examples below, a number of polypeptides having preferred binding characteristics have been identified by one or more of modification schemes (a)-(h), including for example, SEQ ID NOS: 1333-141 as set forth in FIG. 8.

Strategy 3

In another strategy, known polypeptides that bind to IL-23R can be cloned directly on to either the N or C terminal end trimerization domain as free linear pep tides or as disulfide constrained loops using cysteines. Single chain antibodies or domain antibodies capable of binding IL-23R can also be cloned on to either end of the trimerization domain. Additionally peptides with known binding properties can be cloned directly into any one of the loop regions of the TN CTLD. Peptides selected for as disulfide constrained loops or as complementary determining regions of antibodies might be quite amenable to relocation into the loop regions of the CTLD of human tetranectin. For all of these constructs, binding as a monomer, as well as binding and blocking activation as a trimer, when fused with the trimerization domain can then be tested for.

Strategy 4:

In some case direct cloning of peptides with binding activity may not be enough, further optimization and selection may be required. As example, peptides with known binding to IL-23R, such as but not limited to those mentioned above, can be grafted into the CTLD of human tetranectin. In order to select for optimal presentation of these peptides for binding, one or more of the flanking amino acids can be randomized, followed by phage display selection for binding. Furthermore, peptides which alone show limited or weak binding can also be grafted into one of the loops of a CTLD library containing randomization of another additional loop, again followed by selection through phage display for increased binding and/or specificity. Additionally, for peptides identified through crystal structures where the specific interacting/binding amino acids are known, randomization of the non binding amino acids can be explored followed by selection through page display for increased binding and receptor specificity. Regions of the IL-23 ligand identified as being responsible for binding can also be examined across species. Conserved amino acids can be retained while randomization and selection for non species conserved positions can be tested.

Methods of Treatment

Another aspect the invention relates to a method preventing activation of IL-23R in a cell expressing IL-23R. The method includes contacting the cell with an IL-23R binding polypeptide of the invention that includes a trimerizing domain and at least one polypeptide that specifically binds to the IL-23R. In one embodiment of this aspect, the method comprises contacting the cell with a trimeric complex of the invention. The IL-23R binding polypeptide may be an antagonist of IL-23R (or the heterodimeric receptor), or may bind to IL-23R to allow the local delivery of a therapeutic agent associated with the trimerizing domain, as described above, to a tumor, to a site of inflamation or other desired location presenting IL-23R.

In another aspect the invention relates to a method of treating a subject having a an immune disorder or a tumor by administering to the subject a therapeutically effective amount of IL-23R antagonist including polypeptide having a trimerizing domain and at least one polypeptide that specifically binds to the IL-23R. In one embodiment of this aspect, the method comprises administering to the subject a trimeric complex of the invention.

Another aspect of the invention is directed to a combination therapy. Formulations comprising IL-23R antagonists and therapeutic agents are also provided by the present invention. It is believed that such formulations will be particularly suitable for storage as well as for therapeutic administration. The formulations may be prepared by known techniques. For instance, the formulations may be prepared by buffer exchange on a gel filtration column.

IL-23R antagonists and therapeutic agents described herein can be employed in a variety of therapeutic applications. Among these applications are methods of treating various cancers. IL-23R antagonists and therapeutic agents can be administered in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

Effective dosages and schedules for administering the IL-23R antagonists may be determined empirically, and making such determinations is within the skill in the art. Single or multiple dosages may be employed. It is presently believed that an effective dosage or amount of the antagonist used alone may range from about 1 μg/kg to about 100 mg/kg of body weight or more per day. Interspecies scaling of dosages can be performed in a manner known in the art, e.g., as disclosed in Mordenti et al., Pharmaceut. Res., 8:1351 (1991).

When in vivo administration of IL-23R antagonist is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature [see, for example, U.S. Pat. No. 4,657,760; 5,206,344; or 5,225,212]. One of skill will appreciate that different formulations will be effective for different treatment compounds and different disorders, that administration targeting one organ or tissue, for example, may necessitate delivery in a manner different from that to another organ or tissue. Those skilled in the art will understand that the dosage of IL-23R antagonist that must be administered will vary depending on, for example, the mammal which will receive IL-23R antagonist, the route of administration, and other drugs or therapies being administered to the mammal.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors or any other agent that enhances susceptibility of cancer cells to killing by IL-23R antagonists which are known in the art.

Preparation and dosing schedules for chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the Apo2L variant, or may be given simultaneously therewith.

The polypeptides of in the invention and therapeutic agents (and one or more other therapies) may be administered concurrently (simultaneously) or sequentially. In particular embodiments, a non natural polypeptide of the invention, or multimeric (e.g., trimeric) complex thereof, and a therapeutic agent are administered concurrently. In another embodiment, a polypeptide or trimeric complex is administered prior to administration of a therapeutic agent. In another embodiment, a therapeutic agent is administered prior to a polypeptide or trimeric complex. Following administration, treated cells in vitro can be analyzed. Where there has been in vivo treatment, a treated mammal can be monitored in various ways well known to the skilled practitioner. For instance, tumor tissues can be examined pathologically to assay for cell death or serum can be analyzed for immune system responses.

Pharmaceutical Compositions

In yet another aspect, the invention relates to a pharmaceutical composition comprising a therapeutically effective amount of the polypeptide of the invention along with a pharmaceutically acceptable carrier or excipient. As used herein, “pharmaceutically acceptable carrier” or “pharmaceutically acceptable excipient” includes any and all solvents, dispersion media, coating, antibacterial and antifungal agents, isotonic and absorption delaying agents, and the like that are physiologically compatible. Examples of pharmaceutically acceptable carriers or excipients include one or more of water, saline, phosphate buffered saline, dextrose, glycerol, ethanol and the like as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the of the antibody or antibody portion also may be included. Optionally, disintegrating agents can be included, such as cross-linked polyvinyl pyrrolidone, agar, alginic acid or a salt thereof, such as sodium alginate and the like. In addition to the excipients, the pharmaceutical composition can include one or more of the following, carrier proteins such as serum albumin, buffers, binding agents, sweeteners and other flavoring agents; coloring agents and polyethylene glycol.

The compositions can be in a variety of forms including, for example, liquid, semi-solid and solid dosage forms, such as liquid solutions (e.g. injectable and infusible solutions), dispersions or suspensions, tablets, pills, powders, liposomes and suppositories. The preferred form will depend on the intended route of administration and therapeutic application. In an embodiment the compositions are in the form of injectable or infusible solutions, such as compositions similar to those used for passive immunization of humans with antibodies. In an embodiment the mode of administration is parenteral (e.g., intravenous, subcutaneous, intraperitoneal, intramuscular). In an embodiment, the polypeptide (or trimeric complex) is administered by intravenous infusion or injection. In another embodiment, the polypeptide or trimeric complex is administered by intramuscular or subcutaneous injection.

Other suitable routes of administration for the pharmaceutical composition include, but are not limited to, rectal, transdermal, vaginal, transmucosal or intestinal administration.

Therapeutic compositions are typically sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e. polypeptide or trimeric complex) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An article of manufacture such as a kit containing IL-23R antagonists and therapeutic agents useful in the treatment of the disorders described herein comprises at least a container and a label. Suitable containers include, for example, bottles, vials, syringes, and test tubes. The containers may be formed from a variety of materials such as glass or plastic. The label on or associated with the container indicates that the formulation is used for treating the condition of choice. The article of manufacture may further comprise a container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a container with another active agent as described above.

Typically, an appropriate amount of a pharmaceutically-acceptable salt is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of IL-23R antagonist and therapeutic agent.

Therapeutic compositions can be prepared by mixing the desired molecules having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is readily accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated intravenous (i.v.), subcutaneous (s.c.), intramuscular (i.m.) injections or infusions, or as aerosol formulations suitable for intranasal or intrapulmonary delivery (for intrapulmonary delivery see, e.g., EP 257,956).

The molecules disclosed herein can also be administered in the form of sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 1333,988).

Production of Polypeptides

The polypeptide of the invention can be expressed in any suitable standard protein expression system by culturing a host transformed with a vector encoding the polypeptide under such conditions that the polypeptide is expressed. Preferably, the expression system is a system from which the desired protein may readily be isolated. As a general matter, prokaryotic expression systems are are available since high yields of protein can be obtained and efficient purification and refolding strategies. Thus, selection of appropriate expression systems (including vectors and cell types) is within the knowledge of one skilled in the art. Similarly, once the primary amino acid sequence for the polypeptide of the present invention is chosen, one of ordinary skill in the art can easily design appropriate recombinant DNA constructs which will encode the desired amino acid sequence, taking into consideration such factors as codon biases in the chosen host, the need for secretion signal sequences in the host, the introduction of proteinase cleavage sites within the signal sequence, and the like.

In one embodiment the isolated polynucleotide encodes a polypeptide that specifically binds IL-23R and a trimerizing domain. In an embodiment the isolated polynucleotide encodes a first polypeptide that specifically binds IL-23R, and a trimerizing domain. In certain embodiments, the polypeptide that specifically binds IL-23R and the trimerizing domain are encoded in a single contiguous polynucleotide sequence (a genetic fusion). In other embodiments, polypeptide that specifically binds IL-23R and the trimerizing domain are encoded by non-contiguous polynucleotide sequences. Accordingly, in some embodiments the at least one polypeptide that specifically binds IL-23R and the trimerizing domain are expressed, isolated, and purified as separate polypeptides and fused together to form the polypeptide of the invention.

These recombinant DNA constructs may be inserted in-frame into any of a number of expression vectors appropriate to the chosen host. In certain embodiments, the expression vector comprises a strong promoter that controls expression of the recombinant polypeptide constructs. When recombinant expression strategies are used to generate the polypeptide of the invention, the resulting polypeptide can be isolated and purified using suitable standard procedures well known in the art, and optionally subjected to further processing such as e.g. lyophilization.

Standard techniques may be used for recombinant DNA molecule, protein, and polypeptide production, as well as for tissue culture and cell transformation. See, e.g., Sambrook, et al. (below) or Current Protocols in Molecular Biology (Ausubel et al., eds., Green Publishers Inc. and Wiley and Sons 1994). Purification techniques are typically performed according to the manufacturer's specifications or as commonly accomplished in the art using conventional procedures such as those set forth in Sambrook et al. (Molecular Cloning: A Laboratory Manual. Cold Spring Harbor Laboratory Press, Cold Spring Harbor, N.Y. (1989), or as described herein. Unless specific definitions are provided, the nomenclature utilized in connection with the laboratory procedures, and techniques relating to molecular biology, biochemistry, analytical chemistry, and pharmaceutical/formulation chemistry described herein are those well known and commonly used in the art. Standard techniques can be used for biochemical syntheses, biochemical analyses, pharmaceutical preparation, formulation, and delivery, and treatment of patients.

It will be appreciated that a flexible molecular linker optionally may be interposed between, and covalently join, the specific binding member and the trimerizing domain. In certain embodiments, the linker is a polypeptide sequence of about 1-20 amino acid residues. The linker may be less than 10 amino acids, most preferably, 5, 4, 3, 2, or 1. It may be in certain cases that 9, 8, 7 or 6 amino acids are suitable. In useful embodiments the linker is essentially non-immunogenic, not prone to proteolytic cleavage and does not comprise amino acid residues which are known to interact with other residues (e.g. cysteine residues).

The description below also relates to methods of producing polypeptides and trimeric complexes that are covalently attached (hereinafter “conjugated”) to one or more chemical groups. Chemical groups suitable for use in such conjugates are preferably not significantly toxic or immunogenic. The chemical group is optionally selected to produce a conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

A polyol, for example, can be conjugated to polypeptides of the invention at one or more amino acid residues, including lysine residues, as is disclosed in WO 93/00109, supra. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG.

The average molecular weight of the PEG employed in the pegylation of the Apo-2L can vary, and typically may range from about 500 to about 30,000 daltons (D). Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are nonhomogeneous preparations sold according to average molecular weight. For example, commercially available PEG(5000) preparations typically contain molecules that vary slightly in molecular weight, usually ±500 D. The polypeptide of the invention can be further modified using techniques known in the art, such as, conjugated to a small molecule compounds (e.g., a chemotherapeutic); conjugated to a signal molecule (e.g., a fluorophore); conjugated to a molecule of a specific binding pair (e.g., biotin/streptavidin, antibody/antigen); or stabilized by glycosylation, PEGylation, or further fusions to a stabilizing domain (e.g., Fc domains).

A variety of methods for pegylating proteins are known in the art. Specific methods of producing proteins conjugated to PEG include the methods described in U.S. Pat. Nos. 4,179,337, 4,935,465 and 5,849,535. Typically the protein is covalently bonded via one or more of the amino acid residues of the protein to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, polypropylene glycol) and polyethylene-polypropylene glycol copolymers, can be employed using the techniques for conjugation described herein for PEG. The polyols of the invention include those well known in the art and those publicly available, such as from commercially available sources.

Furthermore, other half-life extending molecules can be attached to the N- or C-terminus of the trimerization domain including serum albumin-binding peptides, IgG-binding peptides or peptides binding to FcRn.

It should be noted that the section headings are used herein for organizational purposes only, and are not to be construed as in any way limiting the subject matter described. All references cited herein are incorporated by reference in their entirety for all purposes.

The Examples that follow are merely illustrative of certain embodiments of the invention, and are not to be taken as limiting the invention, which is defined by the appended claims.

### EXAMPLES

The vectors discussed in the following Examples (pANA) are derived from vectors that have been previously described [See US 2007/0275393]. Certain vector sequences are provided in the Sequence Listing and one of skill will be able to derive vectors given the description provided herein. The pPhCPAB phage display vector (SEQ ID NO: 150) has the gIII signal peptide coding region has been fused with a linker to the hTN sequence encoding ALQT (etc.). The C-terminal end of the CTLD region is fused via a linker to the remaining gIII coding region. Within the CTLD region, nucleotide mutations were generated that did not alter the coding sequence but generated restriction sites suitable for cloning PCR fragments containing altered loop regions. A portion of the loop region was removed between these restriction sites so that all library phage could only express recombinants and not wild-type tetranectin. The murine TN CTLD phage display vectors are similarly designed. Another embodiment of these vectors is pANA27 (SEQ ID NO: 164) in which the gene III C-terminal region has been truncated and the suppressible stop codon at the end of the hTN coding sequence has been altered to encode glutamine. The murine vector pANA28 (SEQ ID NO: 165) was constructed in a similar fashion.

### Example 1

Library Construction

### Mutation and Extension of Loop 1

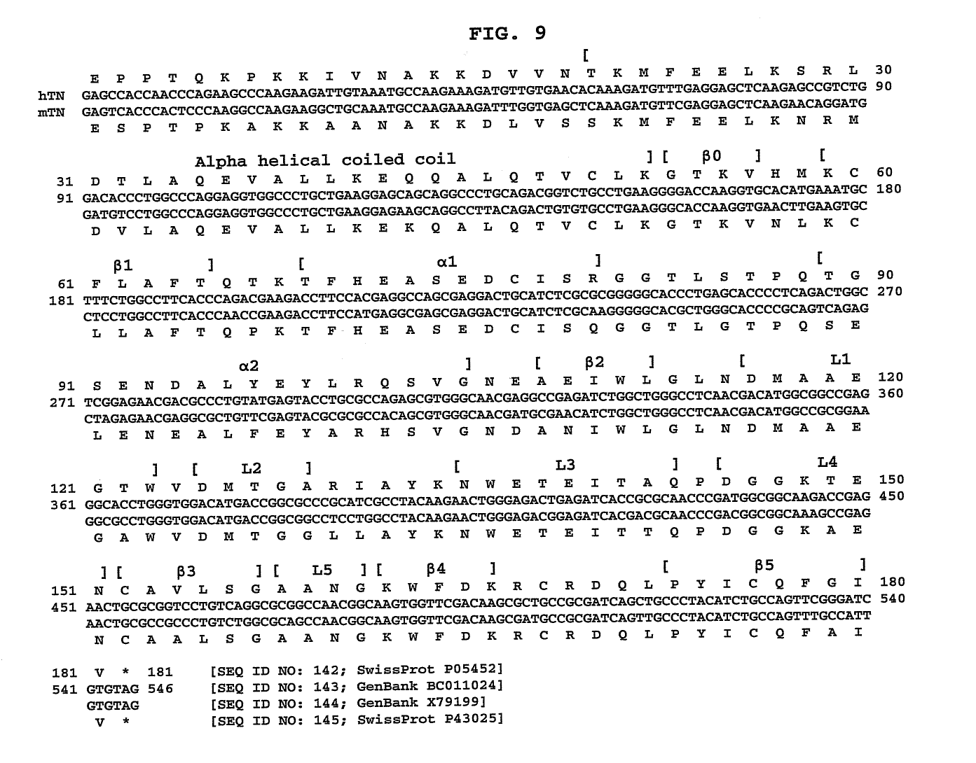

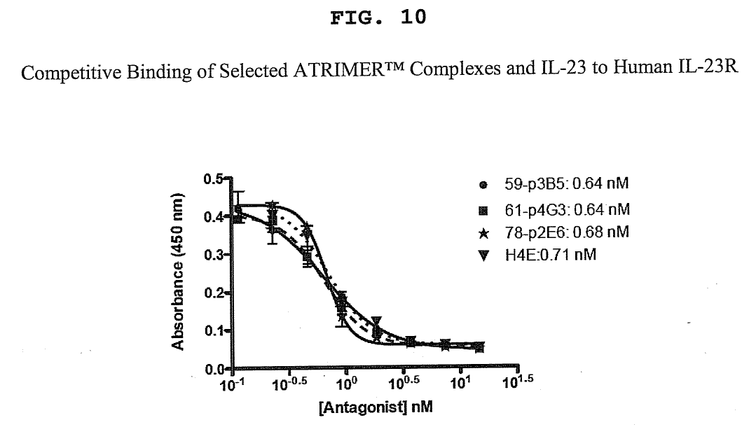

The nucleotide and amino acid sequences of human tetranectin, and the positions of loops 1, 2, 3, 4, and 5 (LSB) are shown in FIG. 9. For the 1-2 extended libraries of human tetranectin C-type lectin binding domains (“Human 1-2X”), the coding sequences for Loop 1 were modified to encode the sequences shown in Table 2, where the five amino acids AAEGT (SEQ ID NO: 469) were substituted with seven random amino acids encoded by the nucleotides NNK NNK NNK NNK NNK NNK NNK (SEQ ID NO: 470); N denotes A, C, G, or T; K denotes G or T. The amino acid arginine immediately following Loop 2 was also fully randomized by using the nucleotides NNK in the coding strand. This amino acid was randomized because the arginine contacts amino acids in Loop 1, and might constrain the configurations attainable by Loop 1 randomization. In addition, the coding sequence for Loop 4 was altered to encode an alanine (A) instead of the Lysine 148 (K) in order to abrogate plasminogen binding, which has been shown to be dependent on the Loop 4 lysine (Graversen et al., 1998).

TABLE 2
Amino acids of loop regions from human tetranectin (TN).
Parentheses indicate neighboring amino acids not
considered part of the loop.
X = any amino acid.

| | | Loop 2 | | | |
|---|---|---|---|---|---|
| | Loop 1 | [SEQ ID | Loop 3 | Loop 4 | Loop |
| Library | [SEQ ID NO] | NO] | [SEQ ID NO] | [SEQ ID NO] | 5 |
| Human | DMAAEGTW | DMTGA(R) | NWETEITAQ(P) | DGGKTEN | AAN |
| TN | [203] | [204] | [205] | [206] | |
| Human | DMXXXXXXXW | DMTGA(X) | NWETEITAQ(P) | DGGATEN | AAN |
| 1-2X | [207] | [208] | [205] | [209] | |
| Human | DMXXXXXW | DMXXX(X) | NWETEITAQ(P) | DGGATEN | AAN |
| 1-2 | [210] | [211] | [205] | [209] | |
| Human | XXXXXXXW | DMTGA(R) | NWETEITAQ(P) | DGGXXXXXEN | AAN |
| 1-4 | [212] | [204] | [205] | [213] | |
| Human | DMAAEGTW | DMTGA(R) | NWXXXXXXQ(P) | DGGATEN | AAN |
| 3X 6 | [203] | [204] | [214] | [209] | |
| Human | DMAAEGTW | DMTGA(R) | NWXXXXXXXQ(P) | DGGATEN | AAN |
| 3X 7 | [203] | [204] | [215] | [209] | |
| Human | DMAAEGTW | DMTGA(R) | NWXXXXXXXXQ(P) | DGGATEN | AAN |
| 3X 8 | [203] | [204] | [216] | [209] | |
| Human | DMAAEGTW | DMTGA(R) | NWETEXXXXXXXTAQ(P) | DGGATEN | AAN |
| 3X loop | [203] | [204] | [217] | [209] | |
| Human | DMAAEGTW | DMTGA(R) | NWETXXXXXXAQ(P) | DGGXXXXXXN | AAN |
| 3-4X | [203] | [204] | [218] | [219] | |
| Human | DMAAEGTW | DMTGA(R) | NWEXXXXXX(X) | XGGXXXN | AAN |
| 3-4 | [203] | [204] | [220] | [221] | |
| combo | | | | | |
| Human | DMAAEGTW | DMTGA(R) | NWEXXXXXQ(P) | DGGATEN | XXX |
| 3-5 | [203] | [204] | [222] | [209] | |
| Human | DMAAEGTW | DMTGA(R) | NWETEITAQ(P) | DGGXXXXXXXN | AAN |
| 4 | [203] | [204] | [205] | [223] | |

The human Loop 1 extended library was generated using overlap PCR in the following manner (primer sequences are shown in Table 3). Primers 1X for (SEQ ID NO: 224) and 1Xrev (SEQ ID NO: 225) were mixed and extended by PCR, and primers BstX1for (SEQ ID NO: 227) and PstBssRevC (SEQ ID NO: 228) were mixed and extended by PCR. The resulting fragments were purified from gels, and mixed and extended by PCR in the presence of the outer primers Bglfor12 (SEQ ID NO: 229) and PstRev (SEQ ID NO: 230). The resulting fragment was gel purified and cut with Bgl II and Pst I and cloned into a phage display vector pPhCPAB or pANA27. The phage display vector pPhCPAB was derived from pCANTAB (Pharmacia), and contained a portion of the human tetranectin CTLD fused to the M13 gene III protein. The CTLD region was modified to include BglII and PstI restriction enzyme sites flanking Loops 1-4, and the 1-4 region was altered to include stop codons, such that no functional gene III protein could be produced from the vector without ligation of an in-frame insert. pANA27 was derived from pPhCPAB by replacing the BamHI to ClaI regions with the BamHI to ClaI sequence of SEQ ID NO: 164 (pANA27). This replaces the amber suppressible stop codon with a glutamine codon and truncates the amino terminal region of gene III.

Ligated material was transformed into electrocompetent XL1-Blue E. coli (Stratagene) and four to eight liters of cells were grown overnight and DNA isolated to generate a master library DNA stock for panning A library size of 1.5×10⁸ was obtained, and clones examined showed diversified sequence in the targeted regions.

TABLE 3
Sequences used in the generation of phage displayed C-type
lectin domain libraries.
M = A or C; N = A, C, G, or T; K = G or T; S = G or C;
W = A or T.

| | | SEQ ID |
|---|---|---|
| Name | Sequence | NO |
| 1Xfor | GGCTGGGCCT GAACGACATG NNKNNKNNKN NKNNKNNKNN KTGGGTGGAT | 224 |
| | ATGACTGGCG CC | |
| 1Xrev | GGCGGTGATC TCAGTTTCCC AGTTCTTGTA GGCGATMNNG GCGCCAGTCA | 225 |
| | TATCCACCCA | |
| 1Xrev2 | GGC GGT GAT CTC AGT TTC CCA GTT CTT GTA GGC GAT GCG | 226 |
| | GGC GCC AGT CAT ATC CAC CCA | |
| BstX1for | ACTGGGAAAC TGAGATCACC GCCCAACCTG ATGGCGGCGC AACCGAGAAC | 227 |
| | TGCGCGGTCC TG | |
| PstBssRev | CCCTGCAGCG CTTGTCGAAC CACTTGCCGT TGGCGGCGCC AGACAGGACC | 228 |
| C | GCGCAGTTCT | |
| Bg1for12 | GCCGAGATCT GGCTGGGCCT GAACGACATG | 229 |
| PstRev | ATCCCTGCAG CGCTTGTCGA ACC | 230 |
| 1-2 for | GGCTGGGCCT GAACGACATG NNKNNKNNKN NKNNKTGGGT GGATATGNNK | 231 |
| | NNKNNKNNKA TCGCCTACAA GAACTGGGA | |
| 1-2 rev | GACAGGACGG CGCAGTTCTC GGTTGCGCCG CCATCAGGTT GGGCGGTGAT | 232 |
| | CTCAGTTTCC CAGTTCTTGT AGGCGAT | |
| PstRev12 | ATCCCTGCAG CGCTTGTCGA ACCACTTGCC GTTGGCGGCG CCAGACAGGA | 233 |
| | CGGCGCAGTT CTC | |
| Bg1Bssfor | GAGATCTGGC TGGGCCTCAA CNNSNNSNNS NNSNNSNNSN NSTGGGTGGA | 234 |
| | CATGACTGGC | |
| BssBg1rev | TTGCGCGGTG ATCTCAGTCT CCCAGTTCTT GTAGGCGATA CGCGCGCCAG | 235 |
| | TCATGTCCAC CCA | |
| BssPstfor | GACTGAGATC ACCGCGCAAC CCGATGGCGG CNNSNNSNNS NNSNNSGAGA | 236 |
| | ACTGCGCGGT CCTG | |
| PstBssRev | CCCTGCAGCG CTTGTCGAAC CACTTGCCGT TGGCCGCGCC TGACAGGACC | 237 |
| | GCGCAGTTCT | |
| Bg1for | GCCGAGATCT GGCTGGGCCT CA | 238 |
| H Loop 1- | ATCTGGCTGG GCCTGAACGA CATGGCCGCC GAGGGCACCT GGGTGGATAT | 239 |
| 2-F | GACCGGCGCG CGTATCGCCT ACAAGAAC | |
| H Loop 3- | CCGCCATCGG GTTGGGCMNN MNNMNNMNNM NNMNNAGTTT CCCAGTTCTT | 240 |
| 4 Ext R | GTAGGCGATA CG | |
| H Loop 3- | GCCCAACCCG ATGGCGGCNN KNNKNNKNNK NNKNNKAACT GCGCCGTCCT | 241 |
| 4 Ext-F | GTCTGGC | |
| H Loop 5- | CCTGCAGCGC TTGTCGAACC ACTTGCCGTT GGCGGCGCCA GACAGGACGG | 242 |
| R | CGCA | |
| H Loop 3- | GCCAGACAGG ACGGCGCAGT TMNNMNNMNN GCCGCCMNNM NNMNNMNNMN | 243 |
| 4 Combo R | NMNNMNNMNN TTCCCAGTTC TTGTAGGCGA TACG | |
| H Loop 3- | CCGCCATCGG GTTGGGCGGT GATCTCAGTT TCCCAGTTCT TGTAGGCGAT | 244 |
| R | ACG | |
| H Loop 4 | GCCCAACCCG ATGGCGGCNN KNNKNNKNNK NNKNNKNNKA ACTGCGCCGT | 245 |
| Ext-F | CCTGTCTGGC | |
| HLoop3F 6 | CTGGCGCGCG TATCGCCTAC AAGAACTGGN NKNNKNNKNN KNNKNNKCAA | 246 |
| | CCCGATGGCG GCGCCACCGA GAAC | |
| HLoop3F 7 | CTGGCGCGCG TATCGCCTAC AAGAACTGGN NKNNKNNKNN KNNKNNKNNK | 247 |
| | CAACCCGATG GCGGCGCCAC CGAGAAC | |
| HLoop3F 8 | CTGGCGCGCG TATCGCCTAC AAGAACTGGN NKNNKNNKNN KNNKNNKNNK | 248 |
| | CAACCCGATG GCGGCGCCAC CGAGAAC | |
| HLoop4R | CCTGCAGCGC TTGTCGAACC ACTTGCCGTT GGCGGCGCCA GACAGGACGG | 249 |
| | CGCAGTTCTC GGTGGCGCCG CCATCGGGTT G | |
| H1-3-4R | GACAGGACCG CGCAGTTCTC GCCSMAGWMC CCSAAGCCGC CMNNGGGTTG | 250 |
| | MNNMNNMNNM NNMNNCTCCC AGTTCTTGTA GGCGATACG | |
| PstLoop4 | ATCCCTGCAG CGCTTGTCGA ACCACTTGCC GTTGGCCGCG CCTGACAGGA | 251 |
| rev | CCGCGCAGTT CTCGCC | |
| Loop3AF2 | GAGCGTGGGCAACGAGGCCGAGATCTGGCTGGGCCTCAACGACATGGCCGCCGA | 252 |
| Loop3AR2 | CCAGTTCTTGTAGGCGATACGCGCGCCAGTCATATCCACCCAGGTGCCCTCGGC | 253 |
| | GGCCATGTCGTTGAGG | |
| Loop3BF | ATCGCCTACAAGAACTGGGAGACTGRGNNKNNKNNKNNKNNKNNKNNKACCGCG | 254 |
| | CAACCCGATGGCGGTGCAAC | |
| Loop3BR | CGCTTGTCGAACCACTTGCCGTTGGCGGCGCCAGACAGGACGGCGCAGTTCTCG | 255 |
| | GTTGCACCGCCATCGGGTTG | |
| M 3X OF | GACATGGCCGCGGAAGGC | 256 |
| M 3X OR | GCAGATGTAGGGCAACTGATCTCT | 257 |
| HuBg1for | GCCGAGATCTGGCTGGGCCTGA | 258 |
| GSXX | GCCGAGATCTGGCTGGGCCTCAACGGCAGCNNKNNKNNKNNKWCCTGGGTGGAC | 259 |
| | ATGACTGGC | |
| 090827 | TTGCGCGGTGATCTCAGTCTCCCAGTTCTTGTAGGCGATACGCGCGCCAGTCAT | 260 |
| BssBg1rev | GTCCACCCA | |
| FGVFGfor | GACTGAGATCACCGCGCAACCCGATGGCGGCTTCGGCGTGTTCGGCGAGAACTG | 261 |
| | CGCGGTCCTG | |
| WGVFGfor | GACTGAGATCACCGCGCAACCCGATGGCGGCTGGGGCGTGTTCGGCGAGAACTG | 262 |
| | CGCGGTCCTG | |
| FGYFGfor | GACTGAGATCACCGCGCAACCCGATGGCGGCTTCGGGTACTTCGGCGAGAACTG | 263 |
| | CGCGGTCCTG | |
| WGYFGfor | GACTGAGATCACCGCGCAACCCGATGGCGGCTGGGGGTACTTCGGCGAGAACTG | 264 |
| | CGCGGTCCTG | |
| WGVWGfor | GACTGAGATCACCGCGCAACCCGATGGCGGCTGGGGCGTGTGGGGCGAGAACTG | 265 |
| | CGCGGTCCTG | |
| h3-5AF | TGGGCCTGAACGACATGGCCGCCGAGGGCACCTGGGTGGATATGACTGGCGCGC | 266 |
| | GTATCGCCTACAAGAACTGGGAG | |
| h3-5AR | GTTGCGCCGCCATCGGGTTGMNNMNNMNNMNNMNNCTCCCAGTTCTTGTAGGCG | 267 |
| | ATACG | |
| h3-5BF | CAACCCGATGGCGGCGCAACCGAGAACTGCGCCGTCCTGTCTGG | 268 |
| h3-5BR | TGTAGGGCAATTGATCCCTGCAGCGCTTGTCGAACCACTTGCCMNNMNNMNNGC | 269 |
| | CAGACAGGACGGCGCAGTT | |
| h3-5 OF | GCCGAGATCTGGCTGGGCCTGAACGACATGG | 270 |

### Example 2

### Library Construction

### Mutation of Loops 1 and 2

For the Loop 1-2 libraries of human tetranectin C-type lectin binding domains (“Human 1-2”), the coding sequences for Loop 1 were modified to encode the sequences shown in Table 2, where the five amino acids AAEGT (SEQ ID NO: 469; human) were replaced with five random amino acids encoded by the nucleotides NNK NNK NNK NNK NNK ((SEQ ID NO: 471); N denotes A, C, G, or T; K denotes G or T). In Loop 2 (including the neighboring arginine), the four amino acids TGAR in human were replaced with four random amino acids encoded by the nucleotides NNK NNK NNK NNK (SEQ ID NO: 472). In addition, the coding sequence for Loop 4 was altered to encode an alanine (A) instead of the lysine (K) in the loop, in order to abrogate plasminogen binding, which has been shown to be dependent on the Loop 4 lysine (Graversen et al., 1998).

The human 1-2 library was generated using overlap PCR in the following manner (primer sequences are shown in Table 3). Primers 1-2 for (SEQ ID NO: 231) and 1-2 rev (SEQ ID NO: 232) were mixed and extended by PCR. The resulting fragment was purified from gels, mixed and extended by PCR in the presence of the outer primers Bglfor12 (SEQ ID NO: 229) and PstRev12 (SEQ ID NO: 233). The resulting fragment was gel purified and cut with Bgl II and Pst I and cloned into similarly digested phage display vector pPhCPAB or pANA27, as described above. A library size of 4.86×10⁸ was obtained, and clones examined showed diversified sequence in the targeted regions.

### Example 3

### Library Construction

### Mutation and Extension of Loops 1 and 4

For the Loop 1-4 library of human C-type lectin binding domains (“Human 1-4”), the coding sequences for Loop 1 were modified to encode the sequences shown in Table 2, where the seven amino acids DMAAEGT (SEQ ID NO: 473) for human were substituted with seven random amino acids encoded by the nucleotides NNS NNS NNS NNS NNS NNS NNS (SEQ ID NO: 474) (N denotes A, C, G, or T; S denotes G or C). In addition, the coding sequences for Loop 4 were modified and extended to encode the sequences shown in Table 1, where two amino acids of Loop 4, KT for human, were replaced with five random amino acids encoded by the nucleotides NNS NNS NNS NNS NNS (SEQ ID NO: 475) for human.

The human 1-4 library was generated using overlap PCR in the following manner (primer sequences are shown in Table 3). Primers BglBssfor (SEQ ID NO: 234) and BssBglrev (SEQ ID NO: 235) were mixed and extended by PCR, and primers BssPstfor (SEQ ID NO: 236) and PstBssRev (SEQ ID NO: 237) were mixed and extended by PCR. The resulting fragments were purified from gels, mixed and extended by PCR in the presence of the outer primers Bglfor (SEQ ID NO: 238) and PstRev (SEQ ID NO: 230). The resulting fragment was gel purified and cut with Bgl II and Pst I restriction enzymes, and cloned into similarly digested phage display vector pPhCPAB or pANA27, as described above. A library size of 2×10⁹ was obtained, and12 clones examined prior to panning showed diversified sequence in the targeted regions.

### Example 4

### Library Construction

### Mutation and Extension of Loops 3 and 4

For the Loop 3-4 extended libraries of human C-type lectin binding domains (“Human 3-4X”), the coding sequences for Loop 3 were modified to encode the sequences shown in Table 2, where the three amino acids EIT of human tetranectin were replaced with six random amino acids encoded by the nucleotides NNK NNK NNK NNK NNK NNK (SEQ ID NO: 476) in the coding strand (N denotes A, C, G, or T; K denotes G or T). In addition, in Loop 4, the three amino acids KTE in human were replaced with six random amino acids encoded by the nucleotides NNK NNK NNK NNK NNK NNK (SEQ ID NO: 476).

The human 3-4 extended library was generated using overlap PCR in the following manner (primer sequences are shown in Table 3). Primers H Loop 1-2-F (SEQ ID NO: 239) and H Loop 3-4 Ext-R (SEQ ID NO: 240) were mixed and extended by PCR, and primers H Loop 3-4 Ext-F (SEQ ID NO: 241 and H Loop 5-R (SEQ ID NO: 242) were mixed and extended by PCR. The resulting fragments were purified from gels, and mixed and extended by PCR in the presence of additional H Loop 1-2-F (SEQ ID NO: 239) and H Loop 5-R (SEQ ID NO: 242). The resulting fragment was gel purified and cut with Bgl II and Pst I restriction enzymes, and cloned into similarly digested phage display vector pPhCPAB or pANA27, as described above. A library size of 7.9×10⁸ was obtained, and clones examined showed diversified sequence in the targeted regions.

### Example 5

### Library Construction

### Mutation of Loops 3 and 4 and the Pro Between the Loops

For the Loop 3-4 combo library of human tetranectin C-type lectin binding domains (“Human 3-4 combo”), the coding sequences for loops 3 and 4 and the proline between these two loops were altered to encode the sequences shown in Table 2, where the human sequence TEITAQPDGGKTE (SEQ ID NO: 477) was replaced by the 13 amino acid sequence XXXGGXXX, (SEQ ID NO: 478) where X represents a random amino acid encoded by the sequence NNK (N denotes A, C, G, or T; K denotes G or T).

The human 3-4 combo library was generated using overlap PCR in the following manner (primer sequences are shown in Table 3). Primers H Loop 1-2-F (SEQ ID NO: 239) and H Loop 3-4 Combo-R (SEQ ID NO: 243) were mixed and extended by PCR and the resulting fragment was purified from gels and mixed and extended by PCR in the presence of additional H Loop 1-2-F (SEQ ID NO: 239) and H loop 5-R (SEQ ID NO: 242). The resulting fragment was gel purified and cut with Bgl II and Pst I restriction enzymes, and cloned into similarly digested phage display vector pPhCPAB or pANA27, as described above. A library size of 4.95×10⁹ was obtained, and clones examined showed diversified sequence in the targeted regions.

### Example 6

### Library Construction

### Mutation and Extension of Loop 4

For the Loop 4 extended libraries of human tetranectin C-type lectin binding domains (“Human 4”), the coding sequences for Loop 4 were modified to encode the sequences shown in Table 2, where the three amino acids KTE of human tetranectin were replaced with seven random amino acids encoded by the nucleotides NNK NNK NNK NNK NNK NNK NNK ((SEQ ID NO: 470); N denotes A, C, G, or T; K denotes G or T).

The human 4 extended library was generated using overlap PCR in the following manner (primer sequences are shown in Table 3). Primers H Loop 1-2-F (SEQ ID NO: 239) and H Loop 3-R (SEQ ID NO: 244) were mixed and extended by PCR, and primers H Loop 4 Ext-F (SEQ ID NO: 245) and H Loop 5-R (SEQ ID NO: 242) were mixed and extended by PCR. The resulting fragments were purified from gels, and mixed and extended by PCR in the presence of additional H Loop 1-2-F (SEQ ID NO: 239) and H Loop 5-R (SEQ ID NO: 242). The resulting fragment gel purified and was cut with Bgl II and Pst I restriction enzymes, and cloned into similarly digested phage display vector pPhCPAB or pANA27, as described above. A library size of 2.7×10⁹ was obtained, and clones examined showed diversified sequence in the targeted regions.

### Example 7

### Library Construction

### Mutation with and without Extension of Loop 3

For the Loop 3 altered libraries of human tetranectin C-type lectin binding domains, the coding sequences for Loop 3 were modified to encode the sequences shown in Table 2, where the six amino acids ETEITA (SEQ ID NO: 479) of human were replaced with six, seven, or eight random amino acids encoded by the nucleotides NNK NNK NNK NNK NNK NNK (SEQ ID NO: 476), NNK NNK NNK NNK NNK NNK NNK (SEQ ID NO: 470), and NNK NNK NNK NNK NNK NNK NNK NNK (SEQ ID NO: 480); N denotes A, C, G, or T; and K denotes G or T. In addition, in Loop 4, the three amino acids KTE in human were replaced with six random amino acids encoded by the nucleotides NNK NNK NNK NNK NNK NNK (SEQ ID NO: 476). In addition the coding sequence for loop 4 was altered to encode an alanine (A) instead of the lysine (K) in the loop, in order to abrogate plasminogen binding, which has been shown to be dependent on the loop 4 lysine (Graversen et al., 1998).

The human Loop 3 altered library was generated using overlap PCR in the following manner. Primers HLoop3F6, HLoop3F7, and HLoop3F8 (SEQ ID NOS: 246-248, respectively) were individually mixed with HLoop4R (SEQ ID NO: 249) and extended by PCR. The resulting fragments were purified from gels, and mixed and extended by PCR in the presence of oligos H Loop 1-2F (SEQ ID NO: 239), HuBglfor (SEQ ID NO: 258) and PstRev (SEQ ID NO: 230). The resulting fragments were gel purified, digested with BglI and PstI restriction enzymes, and cloned into similarly digested phage display vector pPhCPAB or pANA27, as above. After library generation, the three libraries were pooled for panning

Alternate Loop Extension of Loop 3

The human loop 3 loop library is generated using overlap PCR in the following manner. Primers Loop3AF2 (SEQ ID NO: 252) and Loop3AR2 (SEQ ID NO: 253) are mixed and extended by PCR, and primers Loop3BF (SEQ ID NO: 254) and Loop3BR (SEQ ID NO: 255) are mixed and extended by PCR. The resulting fragments are purified from gels, mixed, and subjected to PCR in the presence of primers Bgl for (SEQ ID NO: 238) and Loop3OR. Products are digested with Bgl II and Pst I restriction enzymes, and the purified fragments are cloned into similarly digested phage display vector pPhCPAB or pANA27, as above. In addition the coding sequence for loop 4 was altered to encode an alanine (A) instead of the lysine (K) in the loop, in order to abrogate plasminogen binding, which has been shown to be dependent on the loop 4 lysine (Graversen et al., 1998).

### Example 8

### Mutation of Loops 3 and 5

For the loop 3 and 5 altered libraries of human C-type lectin binding domains, the coding sequences for loops 3 and 5 were modified to encode the sequences shown in Table 2, where the five amino acids TEITA (SEQ ID NO: 481) of human were replaced with five amino acids encoded by the nucleotides NNK NNK NNK NNK NNK (SEQ ID NO: 471), and the three amino acids AAN of human were replaced with three amino acids encoded by the nucleotides NNK NNK NNK. In addition the coding sequence for loop 4 was altered to encode an alanine (A) instead of the lysine (K) in the loop, in order to abrogate plasminogen binding, which has been shown to be dependent on the loop 4 lysine (Graversen et al., 1998).

The human loop 3 and 5 altered library was generated using overlap PCR in the following manner. Primers h3-5AF (SEQ ID NO: 266) and h3-5AR (SEQ ID NO: 267) were mixed and extended by PCR, and primers h3-5BF (SEQ ID NO: 268) and h3-5 BR (SEQ ID NO: 269) were mixed and extended by PCR. The resulting fragments were purified from gels, and mixed and extended by PCR in the presence of h3-50F (SEQ ID NO: 270) and PstRev (SEQ ID NO: 230). The resulting fragment was gel purified, digested with Bgl I and Pst I restriction enzymes, and cloned into similarly digested phage display vector pPhCPAB or pANA27 as above.

### Example 9

### Panning & Screening of Human Library 1-4

Phage generated from human library 1-4 were panned on recombinant human IL-23R/Fc chimera (R&D Systems). Screening of these binding panels after three, four, and/or five rounds of panning using an ELISA plate assay identified receptor-specific binders in all cases.

To generate phage for panning, the master library DNA was transformed by electroporation into bacterial strain TG1 (Stratagene). Cells were allowed to recover for one hour with shaking at 37° C. in SOC (Super-Optimal broth with Catabolite repression) medium prior to increasing the volume 10-fold by adding super broth (SB) to a final concentration of 20% glucose and 20 μg/mL carbenicillin. After shaking at 37° C. For one hour, the carbenicillin concentration was increased to 50 μg/mL for another hour, after which 400 mL of SB with 2% glucose and 50 μg/mL carbenicillin were added, along with helper phage M13K07 to a final concentration of 5×10⁹ pfu/mL. Incubation was continued at 37° C. without shaking for 30 minutes, and then with shaking at 100-150 rpm for another 30 min. Cells were centrifuged at 3200 g at 4° C. For 20 minutes, then resuspended in 500 mL SB medium containing 50 μg/mL carbenicillin and 50 μg/mL kanamycin. Cells were grown overnight at room temperature (RT) with shaking at 150 rpm. Phage were isolated by pelleting the bacterial cells by centrifugation at 15,000 g and 4° C. For 20 min. The supernatant was incubated with one-fourth volume (usually 250 mL of supernatant/bottle +62.5 mL PEG solution) of 20% PEG/2.5 M NaCl on ice for 30 min. The phage is pelleted by centrifugation at 15,000 g and 4° C. For 20 min. The phage pellet was resuspended in 1% bovine serum albumin (BSA) in phosphate buffered saline (PBS) containing 0.1% sodium azide (BSA/PBS/azide) and complete mini-EDTA-free protease inhibitors (Roche), prepared according to the manufacturer's instructions. Alternatively, phage was resuspended in Buffer D, containing 0.05% boiled cassein, 0.025% Tween-20, and protease inhibitors. Material was filter-sterilized using Whatman Puradisc 25 mm diameter, 0.2 μm pore size filters.

Phage generated from human library 1-4 were panned on recombinant human IL-23R/Fc chimera (R&D Systems cat #1686-MR). Library panning was performed either using a plate or a bead format. For the plate format, six to eight wells of a 96-well Immulon HB2 ELISA plate were coated with 250-1000 ng/well of carrier-free human IL-23R/Fc in Dulbecco's PBS. Material was incubated on the plate overnight, after which wells were washed three times with PBS, blocking buffer (either 1% BSA/PBS/azide or Buffer C, containing 0.05% boiled casseing and 1% Tween-20) was added, and wells were then incubated for at least 1 hour at 37° C. Additional wells were also treated with blocking buffer at the same time for later absorption of phage binding to blocking buffer.

Three dilutions of the phage preparation were used: undiluted, 1:10, and 1:100 in blocking buffer plus protease inhibitors. In some rounds of panning, recombinant human IgG1 Fc was added to each of the dilutions to a final concentration of 10 μg/mL. Blocking buffer was removed from the “Block Only” (preabsorption to block) wells and the different phage mixtures were incubated in these wells for another hour at 37° C. Aliquots (50 μL) of each phage mixture were transferred to a washed and blocked target well and allowed to incubate for 2 h at 37° C. For the first round of panning, bound phage were washed once with either 1×PBS/0.05% Tween or with Buffer D, and were eluted using glycine buffer, pH 2.2, containing 1 mg/mL BSA. After neutralization with 2 M Tris base (pH 11.5) the eluted phage were incubated for 15 minutes at room temperature with two to four milliliters of TG1 (Stratagene), XL1-Blue (Stratagene), ER2738 (Lucigen or NEB), or SS320 (Lucigen) cells at an optical density of approximately 0.9 measured at 600 nm (0D₆₀₀) in yeast extract-tryptone (YT) medium. Phage were prepared from this infection using the protocol above, but scaled down by about 20% (volume). Phage prepared from eluted phage were subjected to additional rounds of panning. At each round, titers of input and output phage were determined by plating on agar with appropriate antibiotics, and colonies from these plates were used later for screening for binders by ELISA.

Additional rounds of panning were performed as described above, except that in the second round of panning, washes were increased to 5×, and in subsequent rounds, washes were increased to 10×. Three to six rounds of panning were performed. For the final round of panning, phage were not produced after infection; rather, infected bacteria were grown overnight and a maxiprep (Qiagen kit) was prepared from the DNA. Glycerol stocks (15%) of input phage were stored frozen (at −80° C.) from each round.

For the bead panning format, human IL-23R was biotinylated and purified using a Sulfo-NHS micro biotinylation kit (Thermo-Scientific) according to the manufacturer's instructions. Phage were generated for panning from the master library as per the protocol above, except that the phage pellet was resuspended in a casein buffer containing 0.5% boiled casein, 0.025% Tween 20 in PBS with added EDTA-free protease inhibitors (Roche). Using a magnet, streptavidin magnetic beads (2 tubes with 50 μL or 0.5 mg each of Myone T1 Dynabeads (Invitrogen)) were washed several times in 0.5% boiled casein, 1% Tween 20 to remove preservatives. A 150 μL aliquot of the phage prep was preincubated with one tube of beads for 30 min at 37° C. to remove streptavidin binders. The phage prep was then removed from the beads and 1 μg of biotinylated IL-23R was added along with 10 μL of human Fc at 100 μg/mL and incubated for 2 h at 37° C. with rotation. This material was then added to the remaining tube of washed beads and incubated at 37° C. For 30 min. Using the magnetic stand, beads were washed five times with PBS/0.05% Tween. Phage were eluted with glycine, pH 2.0, neutralized, and used to infect bacteria as described above. In subsequent rounds of panning, bead-bound phage were washed ten times prior to elution. Titers of input and output phage were determined as described above.

For ELISA screening, colonies from later rounds of panning were grown in YT medium with 2% glucose and antibiotics overnight, and an aliquot of each was then used to start fresh cultures that were grown to an OD₆₀₀ of 0.5. Helper phage were added to 5×10⁹ pfu/mL and allowed to infect for 30 min at 37° C., followed by growth at 37° C. with agitation. Bacteria were centrifuged and resuspended in YT medium with carbenicillin and kanamycin and grown overnight for phage production. Bacteria were then pelleted and the medium was removed and mixed with one-fifth volume (1:5 milk mixture:supernatant) of 6×PBS, 18% milk. ELISA plates were prepared by incubating overnight at 4° C. with 50-100 μL of PBS containing 75-100 ng/well of recombinant human IL-23R/Fc. A duplicate plate coated with human IgG Fc (R&D Systems) was used as a control. Plates were washed 3 times with PBS, blocked for 1 h at 37° C. with 3% milk in 1×PBS, and incubated for 1 hour with 100 uL/well of each milk-treated phage mixture. Plates were washed once with PBS/0.05% Tween 20 and twice with PBS, incubated for one hour with an HRP-conjugated anti-M13 antibody (GE Healthcare), washed three times each with PBS/Tween and PBS, and incubated with TMB substrate (VWR). Sulfuric acid was added to stop the color reaction and absorbance was read at 450 nm to identify positive binders.

Binders to human IL-23R were identified from the third and fourth rounds of panning Examples of the sequences from the randomized regions of Loops 1 and 4 from phage-displayed CTLD binders to human IL-23R/Fc chimera are given in Table 4. Examination of these data suggests that for 31/36 of the binders, a motif was evident in the randomized region of Loop 4: the second and fifth amino acids were always glycine, the fourth amino acid was always one of the cyclic amino acids tryptophan or phenylalanine, the first amino acid was hydrophobic, and usually a cyclic amino acid, such as phenylalanine, tyrosine, or tryptophan, and the third amino acid was hydrophobic, and was usually valine. The Loop 1 region had less of a consensus, though glycine and serine appeared predominantly in the first and second positions, and valine was often in the seventh position. Five additional binders did not appear to have this consensus, though two of these probably formed another small group, with MFGMG (SEQ ID NO: 318) or LFGRG (SEQ ID NO: 320) in the Loop 4 region. Many binders were each represented by multiple clones.

TABLE 4
Sequences of human Loop 1 and 4 binders
to human IL-23R/Fc chimera

| | | Loop 1 | | Loop 4 |
|---|---|---|---|---|
| | Loop 1 | SEQ ID | Loop 4 | SEQ ID |
| Clone ID | Sequence | NO | Sequence | NO |
| 001-91.A1A | GSNVTQT | 271 | FGAFG | 272 |
| 001-91.Al2C | GSSVSDV | 273 | FGMWG | 274 |
| 001-69.4H1 | AGRYSLI | 275 | FGVFG | 276 |
| 001-69.4G8 | GSRRSGV | 277 | FGVFG | 276 |
| 001-69.3E5 | RGATVKV | 278 | FGVFG | 276 |
| 001-87.A8E | ANPAQDL | 279 | FGVWG | 280 |
| 001-89.C3G | APGAMEF | 281 | FGVWG | 280 |
| 001-89.C10B | GSPDLGV | 282 | FGVWG | 280 |
| 001-87.A5F | GSVRSAT | 283 | FGYFG | 284 |
| 001-91.Al2E | GSPVGDM | 285 | IGVWG | 286 |
| 001-91.A7F | GSSKLGL | 287 | IGVWG | 286 |
| 001-69.4D4 | GSVRGRT | 288 | IGVWG | 286 |
| 001-69.3C2 | TNVTRTL | 289 | LGVWG | 290 |
| 001-87.A9E | GSALTNT | 291 | LGYWG | 290 |
| 001-89.C3C | ANRRRTM | 292 | MGVWG | 293 |
| 001-91.A7C | GSSVSGL | 294 | VGVFG | 295 |
| 001-69.4C6 | GSWLGDV | 296 | VGVFG | 295 |
| 001-89.C11E | SGKARDV | 297 | VGVFG | 295 |
| 001-91.A3D | GSRFGHL | 298 | WGVFG | 299 |
| 001-89.C3F | GSRISGV | 300 | WGVFG | 299 |
| 001-91.A6B | SGKRRTV | 301 | WGVFG | 299 |
| 001-89.C12C | SGSWART | 302 | WGVFG | 299 |
| 001-69.4C1 | AGARAEY | 303 | WGVWG | 304 |
| 001-69.4F2 | GPGQAGL | 305 | WGVWG | 304 |
| 001-91.A1B | GSTYTDL | 306 | WGVWG | 304 |
| 001-69.4G3 | GTRMTNT | 307 | WGYFG | 308 |
| 001-89.C7F | GSLLTGL | 309 | YGAWG | 310 |
| 001-69.3H4 | GSKAGKL | 311 | YGVFG | 312 |
| 001-69.4C12 | ASLRSRV | 313 | YGVWG | 314 |
| 001-69.4E5 | GNPSGSV | 315 | YGVWG | 314 |
| 001-87.A3B | TGALHQV | 316 | YGVWG | 314 |
| 001-89.C12E | WTKRTAL | 317 | MFGMG | 318 |
| 001-87.A4A | WTLAKNL | 319 | LFGRG | 320 |
| 001-69.4F5 | VLGWRRE | 321 | LVMPM | 322 |
| 001-69.3G5 | LATWLRW | 323 | QRMSY | 324 |
| 001-69.4F9 | QHLGSFW | 325 | VEFQG | 326 |

ELISA assays indicated that these binders did not cross-react with either human IgG1 Fc or with recombinant mouse IL-23R. ELISA and Biacore binding assays indicated that purified monomeric CTLD or full-length trimers from candidate clones 001-69.4G8 and other competed with IL-23 for binding to the human IL-23R. Competitive candidates have been identified that have nanomolar affinities.

### Example 10

### Affinity Maturation of Binders to Human IL-23R

Because the Loop 4 region of the human IL-23R appeared to be a relevant motif, a shuffling approach was developed preserving the diversity of Loop 4 regions already obtained by panning, but resorting them with all possible Loop 1 regions from the original naïve library. To this end, DNA from the round 4 panning of human IL-23R was digested with EcoRI and BssHII restriction enzymes, which cut between the Loop 1 and Loop 4 regions, and a fragment of about 1.4 kb, containing the Loop 4 region, was isolated. Separately, the original human 1-4 library DNA was digested with the same enzymes, and a fragment of about 3.5 kb, containing the Loop 1 region, was isolated. These fragments were ligated together and a new h1-4 shuffle library was generated as described above. The library was panned using the bead protocol (supra), except that at each round of panning the amount of biotinylated recombinant human IL-23R/Fc was decreased about 10-fold, from 200 ng, (to 20 ng, to 2 ng,) to 0.1 ng. Phage supernatants from colonies were screened by ELISA as described above and binders were identified and sequenced. Loop 1 and 4 sequences of the affinity-matured binders appear in Table 5.

TABLE 5
Loop 1 and 4 sequences from affinity-matured
human Loop 1-4 binders to human IL-23R

| | | Loop 1 | | Loop 4 |
|---|---|---|---|---|
| | Loop 1 | SEQ ID | Loop 4 | SEQ ID |
| Clone | Sequence | NO | Sequence | NO |
| 056-40.A3C | GSATTAT | 327 | FGYFG | 284 |
| 056-45.F7F | GSATTDT | 328 | FGYFG | 284 |
| 056-41.B5C | GSALTNT | 291 | FGYFG | 284 |
| 056-53.H7H | GSSVSDV | 273 | FGYFG | 284 |
| 056-53.H4E | GSALTNT | 291 | FGVFG | 276 |
| 056-53.H1G | SGHWRAV | 329 | FGVFG | 276 |
| 056-42.C7D | GSNVTQT | 271 | YGVFG | 312 |
| 056-41.B12F | GSVRSAT | 283 | YGVFG | 312 |
| 056-41.B9B | APPDLGL | 330 | WGVWG | 304 |
| 056-42.C7F | APKSRQY | 331 | FGVWG | 280 |
| 056-44.E4G | VMQLPRK | 332 | IGVWG | 286 |
| 056-53.H7B | AGRMGLV | 333 | WGVFG | 299 |

A separate affinity maturation library was generated in which the diversity of the Loop 1 regions obtained in the initial panning round 4 was maintained, a limited selection of Loop 4 options was utilized, and Loop 3 was randomized in six positions. This was achieved by generating primers to amplify the Loop 1 region using DNA from the original panning round 4 of the human Loop 1-4 library as template, along with primers Bglfor (SEQ ID NO: 238) and H1-3-4R (SEQ ID NO: 250). This primer encodes the following amino acid sequence for loops 3 and 4:

(SEQ ID NO: 482)
RIAYKNWEXXXXXQPXGG(F/L)G(F/Y/V/D)(F/W/L/C)GENCAVL
S.

This sequence incorporates the primary alternatives for Loop 4, as well as alterations of the Loop 3 region of the CTLD. Other primers similar to this but more specific for the Loop 4 region sequences were also generated and used for production of another library randomized in the Loop 3 region. The remainder of the region of interest was generated by overlap PCR using primers PstLoop4rev (SEQ ID NO: 251) and Pst Rev (SEQ ID NO: 230).

Affinity matured IL-23R binding sequences obtained from these libraries are provided in Table 6. Some of the binders obtained were altered by swapping more favorable loop 4 or loop 1 sequences for others to obtain additional affinity-matured binders, and these are included in Table 6.

TABLE 6

| | | SEQ | | SEQ | | SEQ |
|---|---|---|---|---|---|---|
| | | ID | | ID | | ID |
| Clone name | Loop 1 | NO | Loop 3 | NO | Loop 4 | NO |
| H4EP1E9 | GSALTNT | 291 | AGYTKQPS | 334 | FGVFG | 276 |
| H4EWP1E9 | GSALTNT | 291 | AGYTKQPS | 334 | WGVFG | 299 |
| H4EP1E1 | GSALTNT | 291 | LLLRNQPP | 335 | FGVFG | 276 |
| H4EP1D6 | GSALTNT | 291 | QEPAKQPT | 336 | FGVFG | 276 |
| 101-51-1A10 | GSALTNT | 291 | HPLPPQPS | 337 | FGYFG | 284 |
| 101-51-1A3 | GSALTNT | 291 | HQPVYQPG | 338 | WGVFG | 299 |
| 101-54-4B3 | GSALTNT | 291 | LPPPGHPQ | 339 | FGVFG | 276 |
| 101-51-1A5 | GSALTNT | 291 | NGHEPQPR | 340 | FGYFG | 284 |
| 101-51-1A6 | GSALTNT | 291 | NNLSAQPR | 341 | FGYFG | 284 |
| 101-51-1A9 | GSALTNT | 291 | PARQPQPG | 494 | FGYFG | 284 |
| 101-80-5E8 | GSALTNT | 291 | PPEPLHPM | 342 | FGVFG | 276 |
| 101-54-4B6 | GSALTNT | 291 | PPGPHHPM | 343 | FGVFG | 276 |
| 101-113-6C108 | GSALTNT | 291 | PPPPHHPM | 344 | FGVFG | 276 |
| 101-51-1A4 | GSALTNT | 291 | RPALVQPR | 345 | FGVFG | 276 |
| 101-54-4B10 | GSALTNT | 291 | RPPLYQPG | 346 | FGYFG | 284 |
| 101-51-1A7 | GSALTNT | 291 | RPPLYQPG | 346 | WGVFG | 299 |
| 121-26-1A7F | GSALTNT | 291 | RPPLYQPG | 346 | FGVFG | 276 |
| 101-51-1A8 | GSALTNT | 291 | RTPPWQPE | 347 | FGYFG | 284 |
| 101-113-6C102 | GSNVTQT | 271 | PPPPHHPQ | 348 | FGVFG | 276 |
| 101-54-4Al2 | GSRRSGV | 277 | PPGPAHPQ | 349 | FGVFG | 276 |
| 101-113-6A44 | LAGWGMS | 350 | TPPRTQPP | 351 | FGVFG | 276 |
| 101-80-5H3* | GSALTNT | 291 | PPAPYHPM | 352 | -GVFG | 353 |

*Clone 101-80-5H3 had an amino acid deleted from the planned loop 4 and two other amino acid changes (Gly 146, Gly 147 to Ala 146, Ala 147) in the loop 4 region just upstream of the altered region.

Table 7 shows some additional clones that were made with a primer similar to H1-3-4R (SEQ ID NO: 250), but having coding sequences resulting in the selection of the following loop modications.

TABLE 7

| | | SEQ | | SEQ | | SEQ |
|---|---|---|---|---|---|---|
| | | ID | | ID | | ID |
| Clone name | Loop 1 | NO | Loop 3 | NO | Loop 4 | NO |
| 079-86-P1D6h14 | GSTLTRI | 354 | QEPAKQPT | 336 | FGAFG | 272 |
| 079-71-P1E1 | GSALTNT | 291 | LLLRNQPP | 335 | FGAFG | 272 |
| 079-71-PlE9 | GSALTNT | 291 | AGYTKQPS | 334 | LGAFG | 355 |

Another affinity maturation library was generated by limiting loop 4 to five amino acid sequences: FGVFG (SEQ ID NO: 276), WGVFG, FGYFG, WGYFG, and WGVWG (SEQ ID NOS: 299, 284, 308, and 304, respectively), while maintaining the GlySer found at the beginning of loop 1 in IL-23R binders, and varying the subsequent five amino acids in loop 1 using an NNK strategy. Primers GSXX (SEQ ID NO: 259) and 090827 BssBglrev (SEQ ID NO: 260) were mixed and extended using PCR, and primers FGVFGfor, FGYFGfor, WGVFGfor, WGYFGfor, and WGVWGfor (SEQ ID NOS: 261-265) were mixed individually with primer Pst Loop 4 rev (SEQ ID NO: 251) and extended using PCR. The resulting fragments were gel purified and mixed and extended by PCR in the presence of primers Bgl for (SEQ ID NO: 238) and Pst rev (SEQ ID NO: 230). The resulting fragments were digested with Bgl II and Pst I and inserted into vector pANA27 for phage display. Bead panning with successive target dilution was used to select affinity-matured candidates from the library. Sequences of the candidates obtained from this library are provided in Table 8.

TABLE 8

| | | SEQ ID | | SEQ ID |
|---|---|---|---|---|
| Candidate | LOOP 1 | NO: | LOOP 4 | NO: |
| 105-20-1H7 | GSAGTNT | 356 | FGYFG | 284 |
| 105-57-2E8 | GSAHTDT | 357 | WGYFG | 308 |
| 105-08-2G2 | GSAITDT | 358 | WGYFG | 308 |
| 105-08-2B3 | GSAITNT | 359 | WGYFG | 308 |
| 105-20-2C4a | GSAKTDT | 360 | WGYFG | 308 |
| 105-20-1A6 | GSAKTGT | 361 | WGYFG | 308 |
| 105-59-3E5 | GSAKTNT | 362 | WGYFG | 308 |
| 105-08-1C6 | GSALTDT | 363 | FGYFG | 284 |
| 105-08-1D1 | GSALTDT | 363 | WGYFG | 308 |
| 105-20-1B3 | GSALTNT | 291 | FGYFG | 284 |
| 105-59-3H6 | GSALTRT | 364 | WGVFG | 299 |
| 105-59-3C8 | GSALTSL | 365 | WGVWG | 304 |
| 105-57-2D11 | GSARGRV | 366 | WGVWG | 304 |
| 105-20-2F10 | GSARTDT | 367 | FGYFG | 284 |
| 105-08-2D2 | GSARTGT | 368 | FGYFG | 284 |
| 105-08-1D10 | GSARTGT | 368 | WGYFG | 308 |
| 105-08-1A4 | GSAVTNT | 369 | FGYFG | 284 |
| 105-08-2F6 | GSAYTNT | 370 | FGYFG | 284 |
| 105-08-2E12 | GSGLTDT | 371 | WGYFG | 308 |
| 105-55-1A10 | GSGWTGL | 372 | WGVWG | 304 |
| 105-20-2F12 | GSKLTDT | 373 | FGYFG | 284 |
| 105-82-4A3 | GSKVSGL | 374 | WGVFG | 299 |
| 105-08-1D3 | GSKVTET | 375 | FGYFG | 284 |
| 105-61-4D8 | GSLKTDT | 376 | FGVFG | 276 |
| 105-08-2C11 | GSLKTQT | 377 | WGYFG | 308 |
| 105-08-2C10 | GSLLTDT | 378 | FGVFG | 276 |
| 105-08-2G6 | GSLLTDT | 378 | WGYFG | 308 |
| 105-59-3A5 | GSLLTNT | 379 | FGVFG | 276 |
| 105-08-2C4 | GSLLTNT | 379 | FGYFG | 284 |
| 105-61-4B2 | GSLRSDL | 380 | FGVFG | 276 |
| 105-61-4G3 | GSLRTDT | 381 | FGVFG | 276 |
| 105-08-1G12 | GSLRTGT | 382 | WGYFG | 308 |
| 105-78-2D1 | GSLRTHT | 383 | FGVFG | 276 |
| 105-78-2E6 | GSLRTNT | 384 | FGVFG | 276 |
| 105-59-3B9 | GSMLTDT | 385 | FGVFG | 276 |
| 105-08-2A1 | GSMRTDT | 386 | WGYFG | 308 |
| 105-08-2H10 | GSNHTDT | 387 | FGYFG | 284 |
| 105-59-3B5 | GSPITDT | 388 | FGVFG | 276 |
| 105-20-2A3 | GSPITNT | 389 | FGYFG | 284 |
| 105-08-1G9 | GSPKTDT | 390 | FGYFG | 284 |
| 105-08-2G7 | GSPKTGT | 391 | FGYFG | 284 |
| 105-08-2G1 | GSPKTHT | 392 | FGYFG | 284 |
| 105-08-2G10 | GSPLTDT | 393 | FGYFG | 284 |
| 105-61-4G5 | GSPLTNT | 394 | FGVFG | 276 |
| 105-20-1H1 | GSPLTNT | 394 | WGYFG | 308 |
| 105-08-1B7 | GSPRTDT | 395 | FGYFG | 284 |
| 105-08-1A3 | GSPRTDT | 395 | WGVFG | 299 |
| 104-101-1A3F | GSPRTDT | 395 | FGVFG | 276 |
| 105-08-2H11 | GSPRTDT | 395 | WGYFG | 308 |
| 105-08-2H12 | GSPRTET | 396 | FGYFG | 284 |
| 105-08-2G4 | GSPRTGT | 397 | FGYFG | 284 |
| 105-59-3D6 | GSPRTHT | 398 | FGYFG | 284 |
| 105-08-1A8 | GSPRTNT | 399 | FGVFG | 276 |
| 105-20-2G12 | GSPRTNT | 399 | FGYFG | 284 |
| 105-08-1B1 | GSPRTQT | 400 | FGYFG | 284 |
| 105-57-2E11 | GSPRTSV | 401 | FGYFG | 284 |
| 105-08-2H2 | GSPTTDT | 402 | WGYFG | 308 |
| 105-59-3C11 | GSPVNDV | 403 | FGYFG | 284 |
| 105-08-1D2 | GSPVTDT | 404 | FGYFG | 284 |
| 105-55-1F3 | GSPVTDT | 404 | WGYFG | 308 |
| 105-08-2H6 | GSPVTGT | 405 | FGYFG | 284 |
| 105-59-3F1 | GSPVTNT | 406 | FGYFG | 284 |
| 105-59-3H4 | GSQLTDT | 407 | FGYFG | 284 |
| 105-08-1C3 | GSQLTDT | 407 | WGYFG | 308 |
| 105-57-2E2 | GSQLTNT | 408 | FGYFG | 284 |
| 105-08-2C12 | GSQRTDT | 409 | FGYFG | 284 |
| 105-08-2C6 | GSQRTDT | 409 | WGYFG | 308 |
| 105-08-1C2 | GSRATDT | 410 | FGYFG | 284 |
| 105-08-1B10 | GSRHTDT | 411 | FGYFG | 284 |
| 105-76-1D11 | GSRLTDT | 412 | WGVFG | 299 |
| 105-59-3E3 | GSRLTNT | 413 | FGYFG | 284 |
| 105-55-1E3 | GSRRTDT | 414 | FGYFG | 284 |
| 105-20-2G5 | GSRRTDT | 414 | WGYFG | 308 |
| 105-08-1A10 | GSSITDT | 415 | WGYFG | 308 |
| 105-08-1G2 | GSSKTNT | 416 | WGYFG | 308 |
| 105-59-3F9 | GSSLTDT | 417 | FGYFG | 284 |
| 105-08-2C1 | GSSLTDT | 417 | WGYFG | 308 |
| 105-61-4H2 | GSSLTNT | 418 | FGYFG | 284 |
| 105-08-2H3 | GSSLTNT | 418 | WGYFG | 308 |
| 105-08-1C11 | GSSRTDT | 419 | FGYFG | 284 |
| 105-20-1B4 | GSSRTNT | 420 | WGYFG | 308 |
| 105-08-1C10 | GSSVTNT | 421 | WGYFG | 308 |
| 105-82-4A11 | GSSVTST | 422 | WGVFG | 299 |
| 105-08-1C9 | GSTLTDT | 423 | FGYFG | 284 |
| 105-08-1C4 | GSTLTDT | 423 | WGYFG | 308 |
| 105-59-3G12 | GSTLTNT | 424 | FGYFG | 284 |
| 105-08-2C9 | GSTLTNT | 424 | WGYFG | 308 |
| 105-55-1A11 | GSTMTQT | 425 | FGYFG | 284 |
| 105-59-3G9 | GSTRTDT | 426 | FGYFG | 284 |
| 105-59-3B11 | GSTRTNT | 427 | FGYFG | 284 |
| 105-61-4B12 | GSVITGT | 428 | FGYFG | 284 |
| 105-61-4E5 | GSPVTNT | 429 | FGYFG | 284 |
| 105-20-2C4b | GSVKTDT | 430 | WGYFG | 308 |
| 105-08-1D12 | GSVLTDT | 431 | FGYFG | 284 |
| 105-59-3A6 | GSVLTGT | 432 | FGYFG | 284 |
| 105-55-1B9 | GSVLTNT | 433 | FGYFG | 284 |
| 105-08-2H4 | GSVRTDT | 434 | FGYFG | 284 |
| 105-80-3G12 | GSVRTDT | 434 | WGVFG | 299 |
| 105-20-2Cl1 | GSVRTDT | 434 | WGYFG | 308 |
| 105-80-3D4 | GSVRTES | 435 | FGVFG | 276 |
| 105-59-3F11 | GSVRTGT | 436 | FGYFG | 284 |
| 105-08-1A7 | GSVRTNT | 437 | FGYFG | 284 |
| 105-20-2C7 | GSVTTDT | 438 | FGYFG | 284 |
| 105-57-2H2 | GSWGSGI | 439 | WGVWG | 304 |
| 105-08-2C8 | GSWLTDT | 440 | WGYFG | 308 |
| 105-55-1D12 | GSYLTNT | 441 | FGYFG | 284 |

Additional changes in the amino acid sequences of the loops and surrounding sequences were generated by alanine scanning, i.e. the replacement of specific amino acids with the amino acid alanine by means of gene site specific mutagenesis, known to those skilled in the art. Table 9 describes the alanine replacements made in the candidate 056-53.H4E sequence. Such replacements are not limited to the residues shown and can be made in any candidate backbone. Table 10 shows that many of these replacements were beneficial for affinity and/or protein production.

TABLE 9
Sequences of alanine scan candidates that bind IL-23R.

| | | SEQ |
|---|---|---|
| | | ID |
| Candidate | Sequence of AA 115 to 172* | NO. |
| 056-53.H4E | NGSALTNTWVDMTGARIAYKNWETEITAQPDGGFGVFGENCAVLSGAANGKWFDKRCR | 442 |
| H4E N115A | AGSALTNTWVDMTGARIAYKNWETEITAQPDGGFGVFGENCAVLSGAANGKWFDKRCR | 443 |
| H4E G116A | NASALTNTWVDMTGARIAYKNWETEITAQPDGGFGVFGENCAVLSGAANGKWFDKRCR | 444 |
| H4E S117A | NGAALTNTWVDMTGARIAYKNWETEITAQPDGGFGVFGENCAVLSGAANGKWFDKRCR | 445 |
| H4E L119A | NGSAATNTWVDMTGARIAYKNWETEITAQPDGGFGVFGENCAVLSGAANGKWFDKRCR | 446 |
| H4E T120A | NGSALANTWVDMTGARIAYKNWETEITAQPDGGFGVFGENCAVLSGAANGKWFDKRCR | 447 |
| H4E N121A | NGSALTATWVDMTGARIAYKNWETEITAQPDGGFGVFGENCAVLSGAANGKWFDKRCR | 448 |
| H4E T122A | NGSALTNAWVDMTGARIAYKNWETEITAQPDGGFGVFGENCAVLSGAANGKWFDKRCR | 449 |
| H4E W123A | NGSALTNTAVDMTGARIAYKNWETEITAQPDGGFGVFGENCAVLSGAANGKWFDKRCR | 450 |
| H4E R130A | NGSALTNTWVDMTGAAIAYKNWETEITAQPDGGFGVFGENCAVLSGAANGKWFDKRCR | 451 |
| H4E K134A | NGSALTNTWVDMTGARIAYANWETEITAQPDGGFGVFGENCAVLSGAANGKWFDKRCR | 452 |
| H4E N135A | NGSALTNTWVDMTGARIAYKAWETEITAQPDGGFGVFGENCAVLSGAANGKWFDKRCR | 453 |
| H4E W136A | NGSALTNTWVDMTGARIAYKNAETEITAQPDGGFGVFGENCAVLSGAANGKWFDKRCR | 454 |
| H4E E137A | NGSALTNTWVDMTGARIAYKNWATEITAQPDGGFGVFGENCAVLSGAANGKWFDKRCR | 455 |
| H4E T138A | NGSALTNTWVDMTGARIAYKNWEAEITAQPDGGFGVFGENCAVLSGAANGKWFDKRCR | 456 |
| H4E E139A | NGSALTNTWVDMTGARIAYKNWETAITAQPDGGFGVFGENCAVLSGAANGKWFDKRCR | 457 |
| H4E I140A | NGSALTNTWVDMTGARIAYKNWETEATAQPDGGFGVFGENCAVLSGAANGKWFDKRCR | 458 |
| H4E T141A | NGSALTNTWVDMTGARIAYKNWETEIAAQPDGGFGVFGENCAVLSGAANGKWFDKRCR | 459 |
| H4E Q143A | NGSALTNTWVDMTGARIAYKNWETEITAAPDGGFGVFGENCAVLSGAANGKWFDKRCR | 460 |
| H4E D145A | NGSALTNTWVDMTGARIAYKNWETEITAQPAGGFGVFGENCAVLSGAANGKWFDKRCR | 461 |
| H4E G146A | NGSALTNTWVDMTGARIAYKNWETEITAQPDAGFGVFGENCAVLSGAANGKWFDKRCR | 462 |
| H4E G147A | NGSALTNTWVDMTGARIAYKNWETEITAQPDGAFGVFGENCAVLSGAANGKWFDKRCR | 463 |
| H4E E153A* | NGSALTNTWVDMTGARIAYKNWETEITAQPDGGFGVFGANCAVLSGAANGKWFDKRCR | 464 |
| H4E N154A* | NGSALTNTWVDMTGARIAYKNWETEITAQPDGGFGVFGEACAVLSGAANGKWFDKRCR | 465 |
| H4E R170A* | NGSALTNTWVDMTGARIAYKNWETEITAQPDGGFGVFGENCAVLSGAANGKWFDKACR | 466 |
| H4E R172A* | NGSALTNTWVDMTGARIAYKNWETEITAQPDGGFGVFGENCAVLSGAANGKWFDKRCA | 467 |

*Note that the numbering of 056-53.H4E amino acids diverges from the TN sequence numbering in the last four candidates listed, because of the introduction in loop 4 of three additional amino acids. Thus E153 in 056-53.H4E corresponds to E150 in the human TN sequence [7, SEQ ID NO: 131], for example.

TABLE 10
Affinity and production level in E. coli periplasm of 056-53.H4E
ATRIMER ™ polypeptide complexes generated by alanine scanning

| Atrimer | K_{D} (nM) | mg/L |
|---|---|---|
| 056-53.H4E | 0.772 | 1.430 |
| H4E N115A | 7.560 | 0.923 |
| H4E G116A | 10.700 | 1.680 |
| H4E S117A | 2.230 | 1.314 |
| H4E L119A | 1.330 | 1.600 |
| H4E T120A | 1.210 | 1.500 |
| H4E N121A | 0.989 | 1.100 |
| H4E T122A | 6.690 | 1.000 |
| H4E W123A | 11.500 | 1.100 |
| H4E R130A | 1.570 | 1.940 |
| H4E K134A | 1.580 | 0.764 |
| H4E N135A | 1.170 | 0.546 |
| H4E W136A | 14.400 | 0.484 |
| H4E E137A | 0.597 | 1.850 |
| H4E T138A | 0.743 | 2.218 |
| H4E E139A | 0.640 | 1.194 |
| H4E I140A | 1.280 | 1.706 |
| H4E T141A | 0.651 | 1.378 |
| H4E Q143A | 0.689 | 0.444 |
| H4E D145A | 0.714 | 0.876 |
| H4E G146A | 0.960 | 1.092 |
| H4E G147A | 1.030 | 0.512 |
| H4E E153A* | 0.948 | 0.750 |
| H4E N154A* | 0.843 | 1.570 |
| H4E R170A* | 0.777 | 1.984 |
| H4E R172A* | 1.080 | 0.836 |

### Example 11

### Subcloning and Production of CTLD and ATRIMER™ Polypeptide Complex Binders to Human IL-23R

The DNA fragments encoding loop regions were obtained by restriction digestion with BglII and PstI (or MfeI) restriction enzymes, and ligated to the bacterial CTLD expression vectors pANA1, pANA3, or pANA12 that were pre-digested with BglII and PstI. pANA1 (SEQ ID NO: 151) is a T7 based expression vector designed to express C-terminal 6×His-tagged human monomeric CTLD. The pelB signal peptide directs the proteins to the periplasm or growth medium. pANA3 (SEQ ID NO: 153) is the C-terminal HA-His-tagged version of pANA1. pANA12 (SEQ ID NO: 162) is the C-terminal HA-StrepII-tagged version of pANA1. For expression of trimeric protein, the loop regions can be sub-cloned into ATRIMER™ polypeptide complexexpression vectors pANA4 or pANA10 to produce secreted ATRIMER™ polypeptide complexes in E. coli. pANA4 (SEQ ID NO: 154) is a pBAD based expression vector containing C-terminal His/Myc-tagged full length human TN with an ompA signal peptide to direct the proteins to periplasm or growth medium. pANA10 (SEQ ID NO: 160) is the C-terminal HA-StrepII-tagged version of pANA4.

The expression constructs were transformed into E. coli strains BL21(DE3). Star (for pANA1, pANA3 and pANA12; monomeric CTLD production) or BL21(DE3) (for pANA4 and pANA10; ATRIMER™ polypeptide copmlexproduction) were plated on LB/agar plates with appropriate antibiotics. A single colony on a fresh plate was inoculated into 1L of either SB with 1% glucose and kanamycin (for pANA1 and pANA12 vectors) or 2×YT (doubly concentrated yeast tryptone) medium with ampicillin (for pANA4 and pANA10 vectors). The cultures were incubated at 37° C. on a shaker at 200 rpm to an OD₆₀₀ of 0.5, then cooled to room temperature. IPTG was added to a final concentration of 0.05 mM for pANA1 and pANA12, while arabinosis was added to a final concentration of 0.002-0.02% for pANA4 and pANA10. The induction was performed overnight at room temperature with shaking at 120-150 rpm, after which the bacteria were collected by centrifugation. The periplasmic proteins were extracted by osmotic shock or gentle sonication.

The 6×His-tagged proteins were purified using Ni⁺-NTA affinity chromatography. Briefly, periplasmic proteins were reconstituted in a His-binding buffer (100 mM HEPES, pH 8.0, 500 mM NaCl, 10 mM imidazole) and loaded onto a Ni⁺-NTA column pre-equilibrated with His-binding buffer. The column was washed with 10× volume of binding buffer. The bound proteins were eluted with an elution buffer (100 mM HEPES, pH 8.0, 500 mM NaCl, 500 mM imidazole). The purified proteins were dialyzed into 1×PBS buffer and bacterial endotoxin was removed by anion exchange.

The strep II-tagged monomeric CTLDs and ATRIMER™ polypeptide complexes were purified by Strep-Tactin affinity chromatography. Briefly, periplasmic proteins were reconstituted in 1×PBS buffer and loaded onto a Strep-Tactin column pre-equivalent with 1×PBS buffer. The column was washed with 10× volume of PBS buffer. The proteins were eluted with elution buffer (1×PBS with 2.5 mM desthiobiotin). The purified proteins were dialyzed into 1×PBS buffer and bacterial endotoxin was removed by anion exchange.

For some cell assays, ATRIMER™ polypeptide complexes were produced by mammalian cells. DNA fragments encoding loop regions were sub-cloned into the mammalian expression vector pANA2 or pANA11 to produce ATRIMER™ polypeptide complexes in the HEK293 transient expression system. pANA2 (SEQ ID NO: 152) is a modified pCEP4 vector containing a C-terminal His tag. pANA11 (SEQ ID NO: 161) is the C-terminal HA-StrepII-tagged version of pANA2. The DNA fragments encoding loop region were obtained by double digestion with BglII and MfeI and ligated into the expression vectors pANA2 and pANA11 pre-digested with BglII and MfeI. The expression plasmids were purified from bacteria using a Qiagen HiSpeed Plasmid Maxi Kit (Qiagene). For HEK293 adhesion cells, transient transfection was performed using Qiagen SuperFect Reagent according to the manufacturer's protocol. The day after transfection, the medium was removed and changed to 293 Isopro serum-free medium (Irvine Scientific). Two days later, glucose in 0.5 M HEPES buffer was added into the media to a final concentration of 1%. The tissue culture supernatant was collected 4-7 days after transfection for purification. For HEK 293F suspension cells, the transient transfection was performed by Invitrogen's 293Fectin according to the manufacturer's protocol. The next day, 1× volume of fresh medium was added into the culture. The tissue culture supernatant was collected 4-7 days after transfection for purification.

The His or Strep II-tagged ATRIMER™ polypeptide complex purification from mammalian tissue culture supernatant was performed as described for E. coli produced ATRIMER™ polypeptide complexes.

### Example 12

### Characterization of Binders by ELISA and Competition ELISA

ELISA assays, performed as described in Example 9, demonstrated that none of the phage-displayed binders cross-reacted with either human IgG1 Fc or with recombinant mouse IL-23R/Fc (R&D Systems).

Competitive ELISA assays were performed using purified monomeric CTLDs or ATRIMER™ polypeptide complexes generated as described above from positive human IL-23R (IL-23R) binders to block binding of human IL-23 to human IL-23R. Assays were performed generally as follows. Individual wells in Immulon HB2 plates were incubated overnight at 4° C. with 100 μL PBS containing 100 ng of an anti-human IgG Fc (R&D MAB 110 clone 97924). Plates were washed five times with PBS/0.05% Tween 20, and wells were incubated for 1.5 h at RT with 100 μL each of PBS containing 50 ng of recombinant human IL-23R/Fc. Plates were washed as before and blocked for 1 h at RT with 150 μL of 3% bovine serum albumin (Sigma) in PBS, after which plates were washed as described, and wells were incubated for 1-2 hours at RT with 100 μL each of PBS containing IL-23 with or without competitor (ATRIMER™ polypeptide copmlexor CTLD). IL-23-containing solutions were prepared as follows. Human IL-23 (eBioscience) was added at a concentration of 100 ng/mL. Competitor was included at a final concentration of 1 μg/mL. After incubation, plates were washed as described and wells were incubated for 40 min at RT with 100 μL each of PBS containing a 1:5000 dilution of streptavidin-HRP conjugate (Pierce catalog no. 21130). After washing, wells were incubated with 100 μL each of TMB (BioFX Lab catalog no. TMBH-1000-0) for up to 30 min at RT. Reactions were stopped with an equal volume of 0.2 M sulfuric acid.

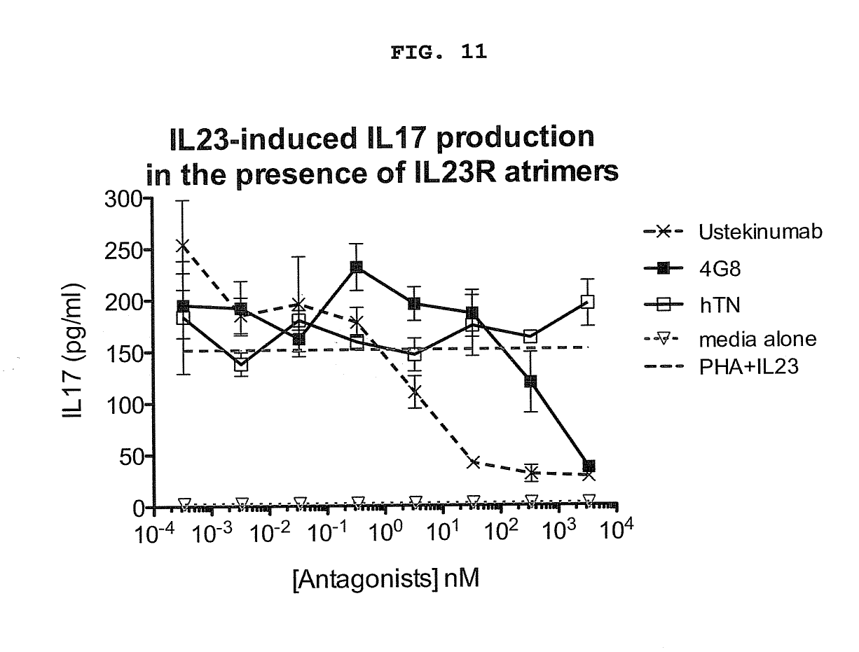

An example of the results of the competition assay (inhibiting IL-23/IL-23R interaction) using the ATRIMER™ polypeptide complexes from the initial panning is presented in FIG. 10. ATRIMER™ polypeptide complexes having the CTLD from clones 59-3B5, 61-p4G3, 78-2E6 and 056-53.H4E from the affinity-matured panning procedure were used in a competition assay with IL-23 for binding to IL-23R.

A number of ATRIMER™ polypeptide complexes were tested in competition ELISA more extensively to determine IC50 values. As shown in Table 11, ATRIMER™ polypeptide complexes displayed low to subnanomolar IC50s.

TABLE 11
Ability of ATRIMER ™ polypeptide complexes to
compete with IL-23 for binding to IL-23R.

| | SEQ ID NOS of | Average IC50 |
|---|---|---|
| hIL-23R binder | Loops 1 & 4 | (nM) |
| H7H | 273, 284 | 0.53 |
| H7B | 333, 299 | 0.9 |
| 4G8 | 277, 276 | 1.4 |
| F7F | 328, 284 | 1.45 |
| B5C | 291, 284 | 1.65 |
| A3C | 327, 284 | 1.8 |
| 056-53.H4E | 291, 276 | 2.5 |
| A9E | 291, 290 | 2.6 |
| H1G | 329, 276 | 3.75 |

The ATRIMER™ polypeptide complex 056-53.H4E was chosen as a standard for comparison, and additional competition assays were performed with affinity-matured ATRIMER™ polypeptide complexes. Table 12 provides the ratio of the 1050 of tested ATRIMER™ polypeptide complexes to that of 056-53.H4E performed in the same assay, in order to better compare competition results among assays.

TABLE 12
Comparison of the ability of ATRIMER ™ polypeptide complexes
to compete with IL-23 for binding to IL-23R.

| | Ratio IC50 to |
|---|---|
| Atrimer | 056-53.H4E IC50 |
| 101-54-4B6 | 0.3 |
| 105-08 1D3 | 0.4 |
| 101-80-5E8 | 0.6 |
| H4E E137A | 0.8 |
| 105-59-3B5 | 0.8 |
| 105-61-4G3 | 0.8 |
| 105-08 2C10 | 0.9 |
| 101-113-6C108 | 0.9 |
| H4E T138A | 1.0 |
| 105-78-2E6 | 1.0 |
| 101-51-1A7 | 1.0 |
| 101-51-1A4 | 1.0 |
| 101-51-1A5 | 1.0 |
| 105-20 2G12 | 1.0 |
| 105-61-4G5 | 1.0 |
| 101-54-4B3 | 1.0 |
| 105-08 1A3 | 1.1 |
| 101-54-4A12 | 1.1 |
| 105-59-3A5 | 1.2 |
| H4E E139A | 1.2 |
| 105-20 2A3 | 1.2 |
| 105-20 1B3 | 1.2 |
| H4E D145A | 1.3 |
| 105-78-2D1 | 1.3 |
| H4E T141A | 1.4 |
| 101-54-4B10 | 1.4 |
| H4E R170A | 1.4 |
| 105-08 1A8 | 1.6 |
| 105-08 1A4 | 1.6 |
| 101-51-1A3 | 1.6 |
| H4E Q143A | 1.6 |
| 105-20 1H1 | 1.8 |
| 105-08 2G10 | 1.8 |
| H4E N154A | 1.9 |
| 101-113-6C102 | 2.0 |
| 105-08 1C6 | 2.0 |
| 105-20 1F3b | 2.0 |
| 105-08 2H6 | 2.0 |
| 105-20 1H7 | 2.1 |
| 101-51-1A9 | 2.2 |
| 105-08 2G1 | 2.2 |
| 105-08 2F6 | 2.4 |
| 105-08 1G9 | 2.4 |
| 105-20 1F3a | 2.5 |
| 105-08 2G7 | 2.5 |
| 105-08 2G4 | 2.5 |
| 101-51-1A6 | 2.6 |
| 105-08 1C11 | 2.8 |
| 105-20 2F12 | 2.8 |
| 105-20 2C4a | 2.9 |
| 105-08 1A7 | 2.9 |
| 105-08 2H3 | 2.9 |
| 105-08 2C4 | 2.9 |
| 105-20 1B4 | 3.0 |
| 105-08 1B1 | 3.3 |
| 105-08 2C12 | 3.3 |
| 105-08 2H12 | 3.3 |
| 105-08 1C4 | 3.3 |
| 105-08 2B3 | 3.4 |
| 105-20 2C7 | 3.5 |
| 105-08 1D1 | 3.6 |
| 105-08 2C1 | 3.6 |
| 105-08 1C3 | 3.6 |
| 105-08 2C6 | 3.6 |
| 101-51-1A8 | 3.7 |
| 105-08 2G2 | 3.8 |
| 105-08 2H2 | 4.0 |
| 105-08 1C2 | 4.1 |
| 105-08 1B7 | 4.1 |
| 105-08 2D2 | 4.1 |
| 105-20 2C4b | 4.2 |
| 105-20 2F10 | 4.2 |
| 105-08 1A10 | 4.3 |
| 105-08 1D2 | 4.3 |
| 105-08 2H11 | 4.3 |
| 105-08 1D12 | 4.6 |
| 105-08 1B10 | 4.7 |
| 105-20 2C11 | 4.8 |
| 105-08 1C10 | 5.0 |
| 105-08 2A1 | 5.0 |
| 105-08 2H4 | 5.0 |
| 105-08 2G6 | 5.2 |
| 105-08 2C9 | 5.3 |
| 105-20 2G5 | 5.3 |
| 105-08 1D10 | 5.5 |
| 105-08 1G2 | 5.5 |
| 105-08 2H10 | 6.5 |
| 105-20 1A6 | 6.6 |
| 105-08 1C9 | 7.4 |
| 105-08 2C8 | 8.4 |
| 101-51-1A10 | 8.7 |
| 105-08 2C11 | 9.1 |
| 105-08 2E12 | 9.1 |
| 101-80-5H3 | 11.3 |
| 105-08 1G12 | 13.2 |

### Example 13

### Characterization of the Affinity of Human IL-23R Binders by Biacore

Apparent affinities of the monomeric and trimeric binders from both the original library panning and the affinity matured library pannings are provided in Tables 13, 14 and 15. A Biacore 3000 biosensor (GE Healthcare) was used to evaluate the interaction of human IL-23R and receptor binders. Immobilization of an anti-human IgG Fc antibody (GE Healthcare) to the CM5 chip (GE Healthcare) was performed using standard amine coupling chemistry, and this modified surface was used to capture a recombinant human IL-23R/Fc fusion protein (R&D Systems). A low-density receptor surface, less than 200 RU, was used for all of the analyses. ATRIMER™ polypeptide complex dilutions (1-500 nM) were injected over the IL-23R surface at 30 μl/min and kinetic constants were derived from the sensorgram data using the Biaevaluation software (version 3.1, GE Healthcare). Data collection was 3 minutes for the association and 5 minutes for dissociation. The anti-human IgG surface was regenerated with a 30s pulse of 3M magnesium chloride. All sensorgrams were double-referenced against an activated and blocked flow-cell as well as buffer injections.

TABLE 13
Affinities of monomeric CTLD IL-23R binders from H Loop 1-4 library

| Analyte | Kₐ (1/M · s) | K_{d} (1/s) | K_{A} (1/M) | K_{D} (nM) |
|---|---|---|---|---|
| A5F | 1.70E+05 | 4.15E−03 | 4.11E+07 | 24.3 |
| 4G8 | 1.43E+05 | 7.83E−03 | 1.83E+07 | 54 |
| B1B | 1.15E+05 | 6.46E−03 | 1.77E+07 | 56.4 |
| A9E | 3.81E+04 | 4.10E−03 | 9.29E+06 | 108 |
| A8E | 5.37E+04 | 7.57E−03 | 7.09E+06 | 141 |
| 4D4 | 2.83E+04 | 4.19E−03 | 6.76E+06 | 148 |
| C7F | 3.58E+04 | 5.31E−03 | 6.75E+06 | 148 |
| C12E | 4.16E+04 | 7.40E−03 | 5.62E+06 | 178 |
| 3C2 | 3.99E+04 | 7.41E−03 | 5.39E+06 | 186 |
| C3C | 8.45E+04 | 1.58E−02 | 5.34E+06 | 187 |
| A4A | 1.18E+05 | 2.29E−02 | 5.18E+06 | 193 |
| 4F5 | 2.35E+04 | 5.71E−03 | 4.12E+06 | 243 |
| B1A | 2.18E+04 | 7.04E−03 | 3.09E+06 | 324 |
| 4E5 | 4.54E+04 | 1.61E−02 | 2.82E+06 | 355 |
| B12C | 1.26E+05 | 5.72E−02 | 2.20E+06 | 455 |
| B7C | 3.03E+04 | 1.99E−02 | 1.52E+06 | 656 |

TABLE 14
Affinities of full-length ATRIMER ™ polypeptide complex IL-23R
binders from the original and the first affinity-matured library.“4G8
TN m” refers to mammalian-cell produced material.
All other material was produced in E. coli.

| Analyte | Kₐ (1/M · s) | K_{d} (1/s) | K_{A} (1/M) | K_{D} (nM) |
|---|---|---|---|---|
| H7B | 4.31E+05 | 2.40E−04 | 1.80E+09 | 0.557 |
| B5C | 3.07E+05 | 3.14E−04 | 9.78E+08 | 1.02 |
| 056-53.H4E | 2.66E+05 | 3.14E−04 | 8.47E+08 | 1.18 |
| F7F | 2.98E+05 | 3.76E−04 | 7.92E+08 | 1.26 |
| H7H | 2.56E+05 | 3.85E−04 | 6.65E+08 | 1.5 |
| A3C | 2.13E+05 | 3.73E−04 | 5.70E+08 | 1.75 |
| A9E | 1.72E+05 | 3.30E−04 | 5.21E+08 | 1.92 |
| B12F | 2.44E+05 | 5.45E−04 | 4.47E+08 | 2.24 |
| A5F | 1.53E+05 | 7.00E−04 | 2.19E+08 | 4.57 |
| 4G8 m | 1.58E+05 | 7.51E−04 | 2.10E+08 | 4.76 |
| H1G | 9.52E+04 | 4.89E−04 | 1.95E+08 | 5.13 |
| B9B | 9.28E+04 | 4.78E−04 | 1.94E+08 | 5.15 |
| C7F | 7.22E+04 | 4.65E−04 | 1.55E+08 | 6.44 |
| 4G8 | 1.09E+05 | 8.05E−04 | 1.35E+08 | 7.42 |
| A4A | 5.06E+04 | 4.09E−04 | 1.24E+08 | 8.08 |
| C3C | 5.79E+04 | 4.83E−04 | 1.20E+08 | 8.34 |
| C6H | 4.95E+04 | 8.45E−04 | 5.85E+07 | 17.1 |

TABLE 15
Affinities of ATRIMER ™ polypeptide complex IL-23R binders from
additional affinity-matured libraries and alanine-scan candidates.
All material was produced in E. coli.

| Analyte | Kₐ (1/M · s) | K_{d} (1/s) | K_{A} (1/M) | K_{D} (nM) |
|---|---|---|---|---|
| 101-113-6C102 | 2.71E+05 | 2.83E−04 | 9.62E+08 | 1.04 |
| 101-113-6C108 | 6.23E+05 | 3.82E−04 | 1.63E+09 | 0.613 |
| 101-51-1A10 | 1.67E+05 | 3.45E−04 | 4.85E+08 | 2.06 |
| 101-51-1A3 | 4.63E+05 | 2.62E−04 | 1.77E+09 | 0.565 |
| 101-51-1A4 | 1.02E+06 | 3.95E−04 | 2.58E+09 | 0.388 |
| 101-51-1A5 | 4.95E+05 | 2.89E−04 | 1.71E+09 | 0.584 |
| 101-51-1A6 | 5.57E+05 | 4.15E−04 | 1.34E+09 | 0.746 |
| 101-51-1A7 | 4.19E+05 | 1.87E−04 | 2.24E+09 | 0.447 |
| 101-51-1A8 | 2.62E+05 | 3.96E−04 | 6.62E+08 | 1.51 |
| 101-51-1A9 | 3.45E+05 | 3.29E−04 | 1.05E+09 | 0.955 |
| 101-54-4A12 | 1.24E+06 | 5.73E−04 | 2.16E+09 | 0.463 |
| 101-54-4B10 | 4.79E+05 | 4.29E−04 | 1.11E+09 | 0.897 |
| 101-54-4B3 | 1.13E+06 | 3.64E−04 | 3.12E+09 | 0.321 |
| 101-54-4B6 | 6.87E+05 | 3.90E−04 | 1.76E+09 | 0.569 |
| 101-80-5E8 | 1.13E+06 | 3.91E−04 | 2.89E+09 | 0.346 |
| 101-80-5H3 | 5.05E+04 | 3.27E−04 | 1.55E+08 | 6.46 |
| 105-08 1A3 | 7.35E+05 | 3.48E−04 | 2.11E+09 | 0.473 |
| 105-08 1A4 | 2.50E+05 | 3.12E−04 | 8.00E+08 | 1.250 |
| 105-08 1A8 | 7.37E+05 | 3.44E−04 | 2.14E+09 | 0.467 |
| 105-08 1D3 | 2.28E+05 | 3.01E−04 | 7.58E+08 | 1.320 |
| 105-08 2C10 | 6.06E+05 | 3.71E−04 | 1.63E+09 | 0.612 |
| 105-08 2F6 | 5.50E+05 | 3.59E−04 | 1.53E+09 | 0.653 |
| 105-08 2G10 | 3.02E+05 | 3.97E−04 | 7.58E+08 | 1.320 |
| 105-08 2G7 | 2.51E+05 | 3.58E−04 | 6.99E+08 | 1.430 |
| 105-20 1B3 | 4.05E+05 | 3.10E−04 | 1.31E+09 | 0.764 |
| 105-20 1H1 | 3.74E+05 | 3.20E−04 | 1.17E+09 | 0.857 |
| 105-20 1H7 | 5.00E+05 | 3.72E−04 | 1.34E+09 | 0.744 |
| 105-20 2A3 | 4.12E+05 | 3.12E−04 | 1.32E+09 | 0.759 |
| 105-20 2F12 | 2.54E+05 | 4.71E−04 | 5.41E+08 | 1.850 |
| 105-20 2G12 | 3.98E+05 | 2.62E−04 | 1.52E+09 | 0.658 |
| H4E D145A | 4.01E+05 | 2.86E−04 | 1.40E+09 | 0.714 |
| H4E E137A | 4.37E+05 | 2.61E−04 | 1.68E+09 | 0.597 |
| H4E E139A | 4.19E+05 | 2.68E−04 | 1.56E+09 | 0.64 |
| H4E N154A | 1.68E+05 | 1.42E−04 | 1.19E+09 | 0.843 |
| H4E Q143A | 3.42E+05 | 2.36E−04 | 1.45E+09 | 0.689 |
| H4E R170A | 3.23E+05 | 2.51E−04 | 1.29E+09 | 0.777 |
| H4E T138A | 3.52E+05 | 2.61E−04 | 1.35E+09 | 0.743 |
| H4E T141A | 4.05E+05 | 2.64E−04 | 1.54E+09 | 0.651 |
| H4EW | 6.51E+05 | 3.64E−04 | 1.79E+09 | 0.560 |

### Example 14

### ATRIMER™ Complexes Binding to IL-23R do not Recognize IL-12Rβ1 or IL-12Rβ2

A Biacore 3000 biosensor (GE Healthcare) was used to evaluate the interaction of human IL-12Rβ1/Fc or IL-12Rβ2/Fc with IL-23R binding ATRIMER™ complexes. Immobilization of an anti-human IgG Fc antibody (GE Healthcare) to the CM5 chip (GE Healthcare) was performed using standard amine coupling chemistry, and this modified surface was used to capture recombinant human IL-12Rβ1/Fc or IL-12Rβ2/Fc fusion protein (R&D Systems). A low-density receptor surface, less than 200 RU, was used for all of the analyses. ATRIMER™ complex dilutions (100 nM) were injected over the IL-12R surface at 30 μl/min. Data collection was 3 minutes for the association and 5 minutes for dissociation. The anti-human IgG surface was regenerated with a 30s pulse of 3M magnesium chloride. All sensorgrams were double-referenced against an anti-human IgG Fc antibody surface as well as buffer injections. As shown in Table 16, ATRIMER™ complexes did not show any measureable binding to human IL-12Rβ1/Fc or IL-12Rβ2/Fc.

TABLE 16

| ATRIMER ™ | | |
|---|---|---|
| (100 nM) | Il12Rb1 | Il12Rb2 |
| 105-08-1A8 | negative | negative |
| H4E-E137A | negative | negative |
| 101-54-4B6 | negative | negative |
| 101-113-6C108 | negative | negative |
| 101-51-1A4 | negative | negative |
| 101-51-1A7 | negative | negative |
| 101-51-1A7F | negative | negative |
| 105-08-1A8 | negative | negative |

### Example 15

### Competitive Assays of Human IL-23 Binding to IL-23R in the Presence of IL-23R Binders USING Biacore

IL-23R binding ATRIMER™ polypeptide complexes were amine-coupled to CM5 chips (GE Healthcare) then IL-23R (IL-23R) was injected over the chip surface. Following binding stabilization, the ability of human IL-23 (eBioscience) to interact with IL-23R was monitored. Additional competition assays were done by pre-forming a complex between IL-23R and IL-23 or IL-23R and ATRIMER™ polypeptide complexes for 30 minutes at room temperature. The complex was then injected over the surface with the amine-coupled ATRIMER™ complexes. Remaining binding of IL-23R Atrimer, as shown in Table 17 for Atrimer A5F was determined and expressed as percent of binding in the absence of competitor (IL-23 or different Atrimer).[

TABLE 17
A5F competes with binding of IL-23 to the IL-23R

| Analyte | Percent binding to A5F |
|---|---|
| rhIL23RFc | 100 |
| rhIL23RFc + rhIL23 | 19 |
| rhIL23RFc + A9E | 25 |

### Example 16

### Testing Activity of Selected ATRIMER™ Polypeptide Complex in Cell Based Assay

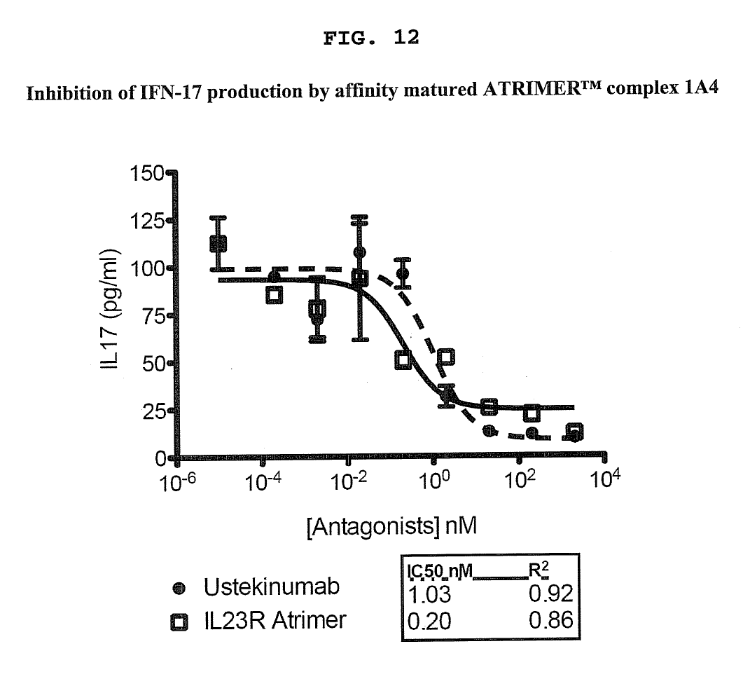

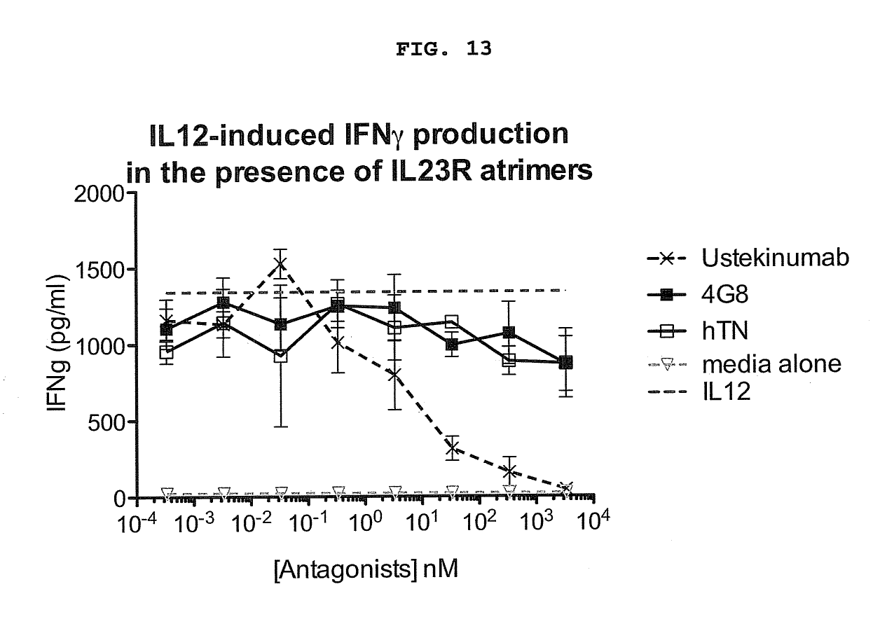

Human peripheral blood mononuclear cells (PBMC) from healthy donors (AllCells) were stimulated at 1×10⁶ cells/mL with human recombinant IL-23 (1 ng/mL, eBioscience) and PHA (1 μg/mL, Sigma) in the presence of IL-23R ATRIMER™ polypeptide complexes or Ustekinumab in 10% FBS/Advanced RPMI media (Invitrogen). After 4 days in culture, cell supernatants were collected and assayed by ELISA using IL-17 Quantikine kits (R&D Systems). In parallel cultures, PBMC were treated with human recombinant IL-12 (1 ng/mL, R&D Systems) in the presence of IL-23R ATRIMER™ polypeptide complexes or Ustekinumab for 4 days. Cell supernatants were assayed for IFNγ and IL-17 by Luminex (Procarta, Panomics) and analyzed on the Bioplex system (BioRad). All treatments were performed in triplicate, and the mean and standard error were plotted using GraphPad Prism software. As shown in FIGS. 11, 12, and 13, IL-23 ATRIMER™ polypeptide complexes blocked IL-23-induced IL-17 production, but did not inhibit IL-12-induced IFNγ production. As expected, Ustekinumab inhibited both IL-23 and IL-12 responses.

Table 18 shows the results for affinity-matured ATRIMER™ polypeptide complexes tested in the PBMC assay. The ability of the ATRIMER™ polypeptide complexes to block IL-23-induced IL-17, IL-17F, and IL-22 production was measured for ATRIMER™ polypeptide complexes as indicated. The results are shown as a ratio with the numerator being the IC50 for the ATRIMER™ polypeptide complexes compared to the IC50 for ustekinumab. Results of more than one assay are shown for some ATRIMER™ polypeptide complexes.

TABLE 18
Production levels of the indicated cytokines in the presence of each
ATRIMER ™ polypeptide complex compared
to ustekinumab in the same experiment.
Atrimer/Ustekinumab

| ATRIMER ™ complex | IL17 | IL-17F | IL22 |
|---|---|---|---|
| 101-113-6C108 | 0.013/1.03 | 0.41/0.77 | |
| 105-08 1A8 | 0.14/0.16 | 0.42/0.1 | |
| 101-51-1A4 | 0.2/1.03 | 4.9/1.05 | 0.27/0.09 |
| | 0.12/0.47 | 0.09/0.25 | |
| 101-54-4B6 | 0.1/0.47 | 0.18/0.25 | 0.12/0.09 |
| | 8.8/0.56 | 5.2/0.55 | |
| | 0.15/0.16 | 0.11/0.1 | |
| H4E E137A | 1.4/0.73 | 2.1/0.34 | |
| | | 16/0.55 | |
| 101-51-1A7 | 1.8/0.58 | 4.4/0.44 | |
| 101-54-4B3 | 3.6/0.16 | 0.16/0.1 | |
| 105-08 2C10 | 3.1/0.47 | 5.2/0.25 | 1.8/0.09 |
| 101-54-4B10 | 4.4/0.93 | 6.6/2.3 | |
| 101-80-5E8 | 7.9/1.03 | 12.9/0.77 | |
| 105-20 1H7 | 16/0.33 | 4.2/0.43 | |
| H4E T138A | 8.8/0.73 | 13/0.34 | |
| 056-53 H4E | 17/0.73 | 45/0.34 | |
| 101-51-1A5 | 34/0.58 | 18/0.44 | |
| 105-08 1B7 | 19/0.93 | 225/2.3 | |
| 105-08 1D3 | 109/0.58 | 31/0.44 | |
| 105-20 2G12 | 158/0.93 | 601/2.3 | |
| 105-08 1A3 | 233/3.0 | 201/3.3 | |

### Example 17

### NKL Agonist Assay

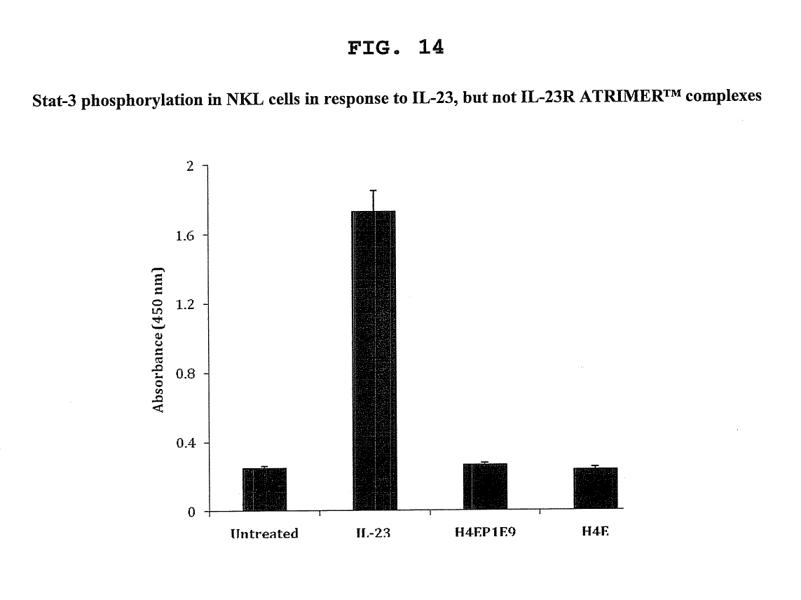

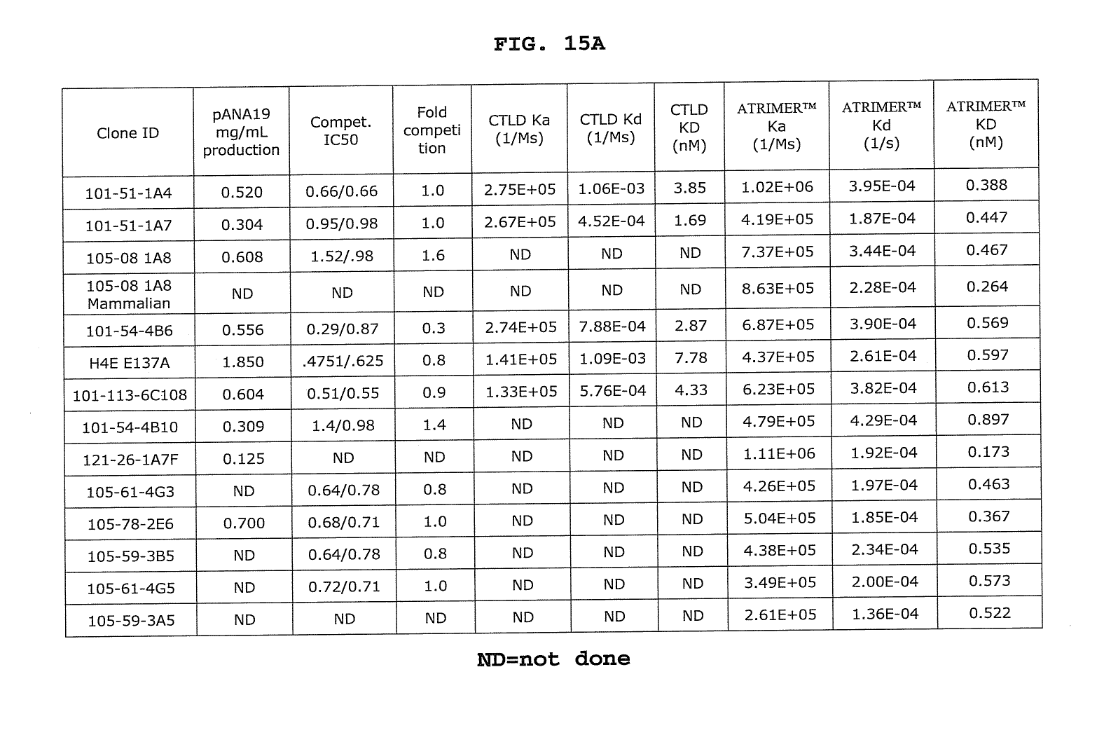

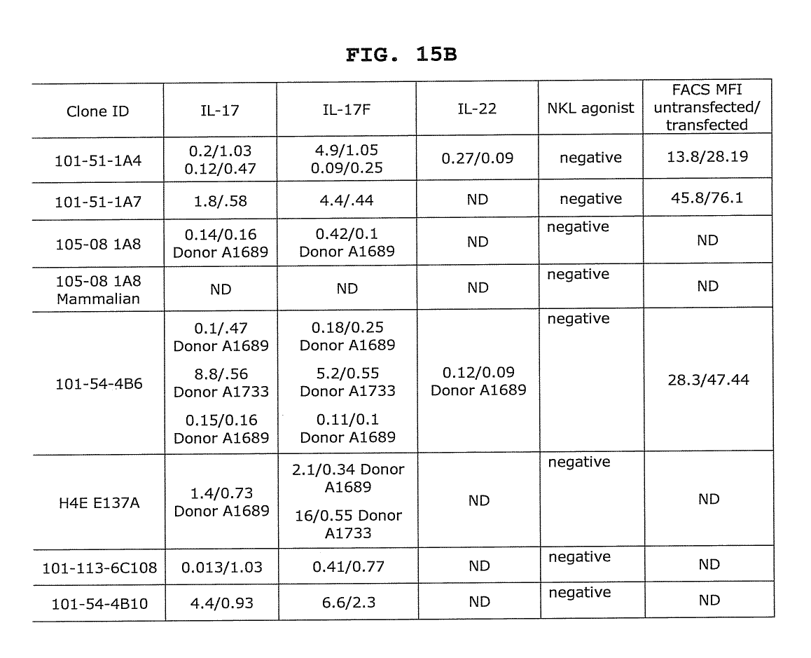

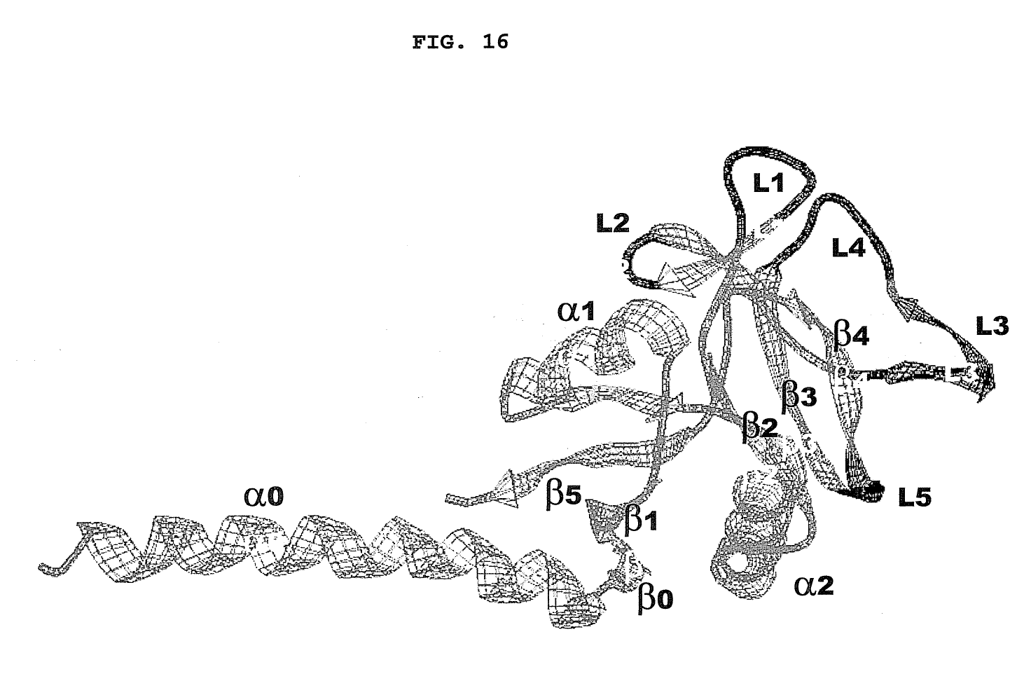

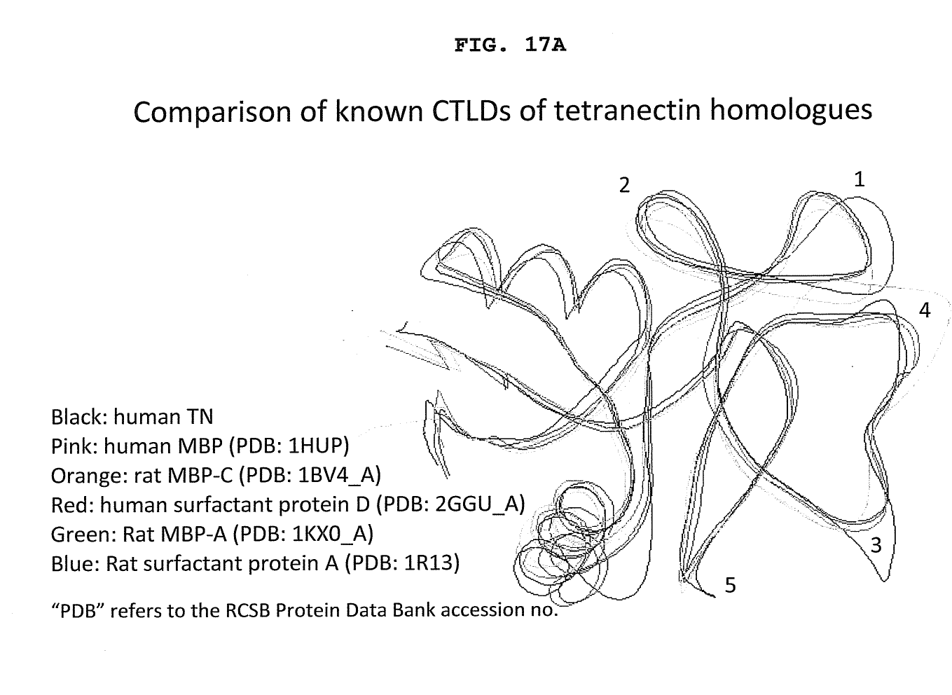

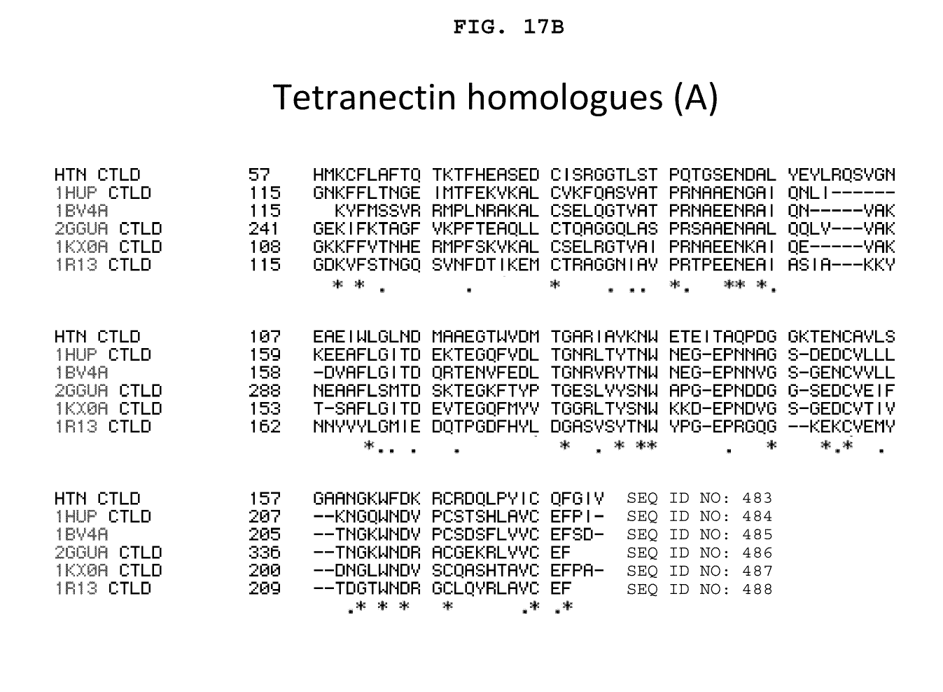

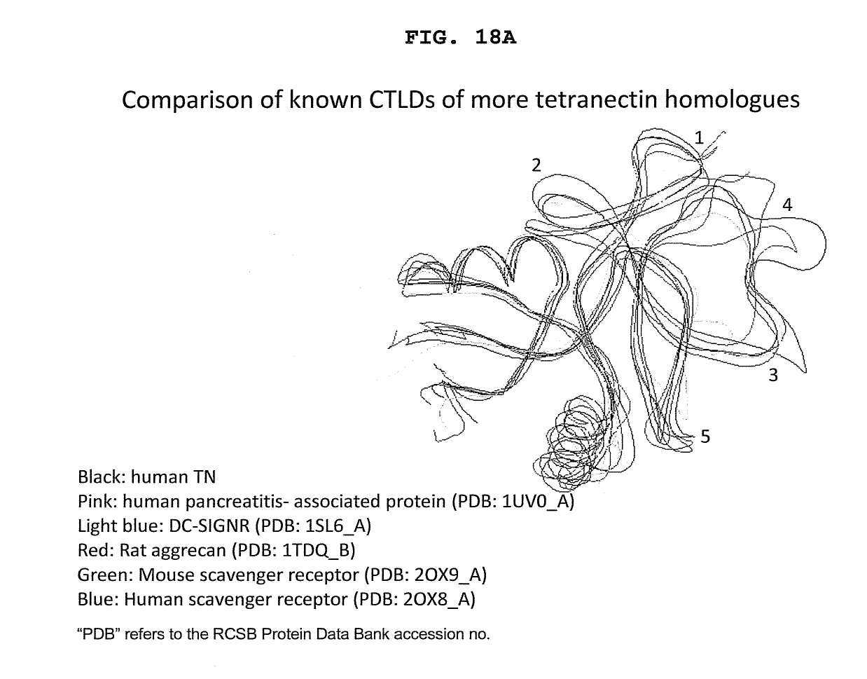

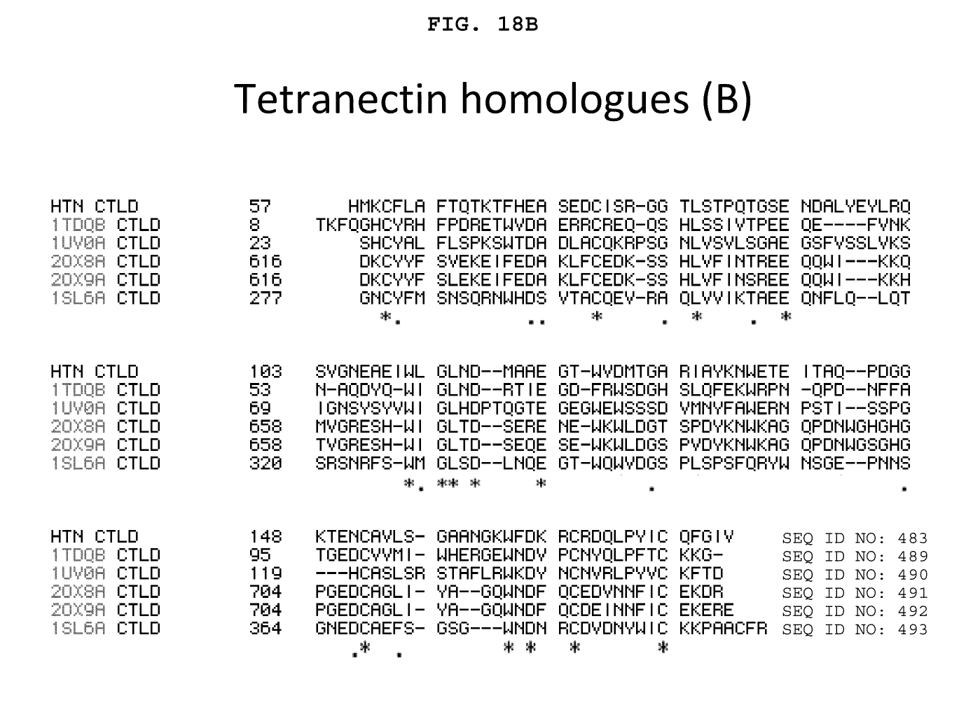

To show the lack of agonist activity of IL-23R ATRIMER™ polypeptide complexes on IL-23R, STAT-3 phosphorylation upon binding of selected IL-23R ATRIMER™ complexes to the natural killer cell line NKL expressing the heterodimeric IL-23 receptor was determined. ATRIMER™ complexes at a concentration of 150 μg/mL or IL-23 at 50 ng/mL as positive control were incubated at 37° C. with 140,000 NKL cells/well in a 96-well plate. After 10 min, cells were centrifuged at 1200 rpm for 5 min, and washed with PBS twice. Then, cells were lysed and treated according to the protocol provided in the Stat3 phosphorylation kit that was obtained from Cell Signaling Technology (PATH SCAN® Phospho Stat3 Sandwich ELISA kit, Cat #7300, Cell Signaling Technology, Inc., Danvers, Mass.). Stat-3 phopshorylation was measured by absorbance at 450 nM using a Molecular Devices ELISA plate reader. As shown in FIG. 14 exemplary for complexes of 056-53.H4E and H4EP1E9, no activation of IL-23R receptor by the ATRIMER™ complexes was observed, while IL-23 resulted in STAT-3 phosphorylation as expected. Similar results were obtained for all other atrimers tested such as 101-51-1A4, 101-51-1A7, 105-08-1A8, 101-54-4B6, H4E E137A, 101-113-6C108 and 101-54-4B10 as summarized in FIGS. 15A and 15B.

The above examples do not limit the scope of variation that can be generated in these libraries. Other libraries can be generated in which varying numbers of random or more targeted amino acids are used to replace existing amino acids, and different combinations of loops can be utilized. In addition, other mutations and methods of generating mutations, such as random PCR mutagenesis, can be utilized to provide diverse libraries that can be subjected to panning

TABLE 19
TAS and TAA sequence information:

| Protein | References |
|---|---|
| AFP | Genbank NM_001134 [Homo sapiens alpha-fetoprotein |
| alfafetoprotein | (AFP), mRNA] |
| alphafetoprotein | Williams et al. (1977), “Tumor-associated antigen levels |
| alpha-fetoprotein | (carcinoembryonic antigen, human chorionic gonadotropin, |
| | and alpha-fetoprotein) antedating the diagnosis of cancer in |
| | the Framingham study.” J. Natl. Cancer Inst. 58(6): 1547-51. |
| CEA | Genbank M29540 [Human carcinoembryonic antigen |
| carcinoembryonic antigen | mRNA (CEA), complete cds] |
| | Williams et al. (1977), “Tumor-associated antigen levels |
| | (carcinoembryonic antigen, human chorionic gonadotropin, |
| | and alpha-fetoprotein) antedating the diagnosis of cancer in |
| | the Framingham study.” J. Natl. Cancer Inst. 58(6): 1547-51. |
| CA-125 | Genbank NM_024690 [Homo sapiens mucin 16, cell |
| cancer antigen 125 | surface associated (MUC16), mRNA] |
| carbohydrate antigen 125 | Boivin et al. (2009), “CA125 (MUC16) tumor antigen |
| also known as | selectively modulates the sensitivity of ovarian cancer cells |
| MUC16 | to genotoxic drug-induced apoptosis.” Gynecol. Oncol., |
| mucin 16 | Sep. 9, Epub ahead of print. |
| MUC1 | Genbank BC120974 [Homo sapiens mucin 1, cell surface |
| mucin 1 | associated, mRNA (cDNA clone MGC: 149467 |
| also known as | IMAGE: 40115473), complete cds] |
| epithelial tumor antigen | Acres and Limacher (2005), “MUC1 as a target antigen for |
| | cancer immunotherapy.” Expert Rev. Vaccines 4(4): 493-502. |
| glypican 3 | Genbank BC035972 [Homo sapiens glypican 3, mRNA |
| | (cDNA clone MGC: 32604 IMAGE: 4603748), complete |
| | cds] |
| | Nakatsura and Nishimura (2005), “Usefulness of the novel |
| | oncofetal antigen glypican-3 for diagnosis of |
| | hepatocellular carcinoma and melanoma.” BioDrugs 19(2): |
| | 71-7. |
| TAG-72 | Lottich et al. (1985), “Tumor-associated antigen TAG-72: |
| tumor-associated glycoprotein | correlation of expression in primary and metastatic breast |
| 72 | carcinoma lesions.” Breast Cancer Res. Treat. 6(1): 49-56. |
| tyrosinase | Genbank BC027179 [Homo sapiens tyrosinase |
| | (oculocutaneous albinism IA), mRNA (cDNA clone |
| | MGC: 9191 IMAGE: 3923096), complete cds] |
| MAA | Genbank BC144138 [Homo sapiens melanoma associated |
| melanoma-associated antigen | antigen (mutated) 1, mRNA (cDNA clone MGC: 177675 |
| | IMAGE: 9052658), complete cds] |
| | Chee et al. (1976), “Production of melanoma-associated |
| | antigen(s) by a defined malignant melanoma cell strain |
| | grown in chemically defined medium.” Cancer Res. 36(4): |
| | 1503-9. |
| MART-1 | Genbank BC014423 [Homo sapiens melan-A, mRNA |
| melanoma antigen recognized by | (cDNA clone MGC: 20165 IMAGE: 4639927), complete |
| T-cells 1 | cds] |
| also known as | Du et al. (2003), “MLANA/MART1 and |
| MLANA | SILV/PMEL17/GP100 are transcriptionally regulated by |
| melan-A | MITF in melanocytes and melanoma.” Am. J. Pathol. |
| | 163(1): 333-43. |
| gp100 | Adema et al. (1994), “Molecular characterization of the |
| | melanocyte lineage-specific antigen gp100.” J. Biol. Chem. |
| | 269(31): 20126-33. |
| | Zhai et al. (1996), “Antigen-specific tumor vaccines. |
| | Development and characterization of recombinant |
| | adenoviruses encoding MART1 or gp100 for cancer |
| | therapy.” J. Immunol. 156(2): 700-10. |
| TRP1 | Genbank AF001295 [Homo sapiens tyrosinase related |
| tyrosinase-related protein 1 | protein 1 (TYRP1) gene, complete cds] |
| | Wang and Rosenberg (1996), “Human tumor antigens |
| | recognized by T lymphocytes: implications for cancer |
| | therapy.” J. Leukoc. Biol. 60(3): 296-309. |
| TRP2 | Genbank L18967 [Homo sapiens TRP-2/dopachrome |
| tyrosinase-related protein 2 | tautomerase (Tyrp-2) mRNA, complete cds] |
| dopachrome tautomerase | Wang et al. (1996), “Identification of TRP-2 as a human |
| | tumor antigen recognized by cytotoxic T lymphocytes.” J. |
| | Exp. Med. 184(6): 2207-16. |
| MSH1 | Genbank NP_011988 [DNA-binding protein of the |
| Note: in yeast only-this protein is | mitochondria involved in repair of mitochondrial DNA, |
| not present in humans. | has ATPase activity and binds to DNA mismatches; has |
| | homology to E. coli MutS; transcription is induced during |
| | meiosis; Msh1p [Saccharomyces cerevisiae]] |
| | Foury et al. (2004), “Mitochondrial DNA mutators.” Cell. |
| | Mol. Life Sci. 61(22): 2799-811. |
| MAGE-1 | Genbank NP_004979 [melanoma antigen family A, 1 |
| MAGEA1 | [Homo sapiens]] |
| melanoma antigen family A 1 | Zakut et al. (1993), “Differential expression of MAGE-1, -2, |
| melanoma-associated antigen 1 | and -3 messenger RNA in transformed and normal |
| | human cell lines.” Cancer Res. 53(1): 5-8. |
| | Eichmuller et al. (2002), “mRNA expression of tumor- |
| | associated antigens in melanoma tissues and cell lines.” |
| | Exp. Dermatol. 11(4): 292-301. |
| MAGE-2 | Genbank L18920 [Human MAGE-2 gene exons 1-4, |
| MAGEA2 | complete cds] |
| melanoma antigen family A 2 | Zakut et al. (1993), “Differential expression of MAGE-1, -2, |
| melanoma-associated antigen 2 | and -3 messenger RNA in transformed and normal |
| | human cell lines.” Cancer Res. 53(1): 5-8. |
| MAGE-3 | Genbank U03735 [Human MAGE-3 antigen (MAGE-3) |
| MAGEA3 | gene, complete cds] |
| melanoma antigen family A 3 | Zakut et al. (1993), “Differential expression of MAGE-1, -2, |
| melanoma-associated antigen 3 | and -3 messenger RNA in transformed and normal |
| | human cell lines.” Cancer Res. 53(1): 5-8. |
| MAGE-12 | Genbank NP_005358 [melanoma antigen family A, 12 |
| MAGEA12 | [Homo sapiens]] |
| melanoma antigen family A 12 | Gibbs et al. (2000), “MAGE-12 and MAGE-6 are |
| melanoma-associated antigen 12 | frequently expressed in malignant melanoma.” Melanoma |
| | Res. 10(3): 259-64. |
| RAGE-1 | Genbank BC053536 [Homo sapiens renal tumor antigen, |
| renal tumor antigen 1 | mRNA (cDNA clone MGC: 61453 IMAGE: 5175851), |
| | complete cds] |
| | Eichmuller et al. (2002), “mRNA expression of tumor- |
| | associated antigens in melanoma tissues and cell lines.” |
| | Exp. Dermatol. 11(4): 292-301. |
| GAGE-1 | Genbank U19141 [Human GAGE-1 protein mRNA, |
| G antigen 1 | complete cds] |
| | Eichmuller et al. (2002), “mRNA expression of tumor- |
| | associated antigens in melanoma tissues and cell lines.” |
| | Exp. Dermatol. 11(4): 292-301. |
| | De Backer et al. (1999), “Characterization of the GAGE |
| | genes that are expressed in various human cancers and in |
| | normal testis.” Cancer Res. 59(13): 3157-65. |
| GAGE-2 | Genbank U19143 [Human GAGE-2 protein mRNA, |
| G antigen 2 | complete cds] |
| | De Backer et al. (1999), “Characterization of the GAGE |
| | genes that are expressed in various human cancers and in |
| | normal testis.” Cancer Res. 59(13): 3157-65. |
| BAGE | Genbank BC107038 [Homo sapiens B melanoma antigen, |
| B melanoma antigen | mRNA (cDNA clone MGC: 129548 IMAGE: 40002186), |
| | complete cds] |
| | Boel et al. (1995), “BAGE: a new gene encoding an |
| | antigen recognized on human melanomas by cytolytic T |
| | lymphocytes.” Immunity 2(2): 167-75. |
| NY-ESO-1 | Genbank BC130362 [Homo sapiens cancer/testis antigen |
| also known as | 1B, mRNA (cDNA clone MGC: 163234 |
| cancer/testis antigen 1B | IMAGE: 40146393), complete cds] |
| | Schultz-Thater et al. (2000), “NY-ESO-1 tumour |
| | associated antigen is a cytoplasmic protein detectable by |
| | specific monoclonal antibodies in cell lines and clinical |
| | specimens.” Br. J. Cancer 8(2): 204-8. |
| beta-catenin | Genbank NM_001098209 [Homo sapiens catenin |
| | (cadherin-associated protein), beta 1, 88 kDa (CTNNB1), |
| | mRNA] |
| CDCP-1 | Genbank BC021099 [Homo sapiens CUB domain |
| CUB domain containing protein 1 | containing protein 1, mRNA (cDNA clone |
| | IMAGE: 4590554), complete cds] |
| | Wortmann et al. (2009), “The cell surface glycoprotein |
| | CDCP1 in cancer--insights, opportunities, and challenges.” |
| | IUBMB Life 61(7): 723-30. |
| CDC-27 | Genbank BC011656 [Homo sapiens cell division cycle 27 |
| cell division cycle 27 homolog | homolog (S. cerevisiae), mRNA (cDNA clone MGC: 12709 |
| | IMAGE: 4301175), complete cds] |
| | Wang et al. (1999), “Cloning genes encoding MHC class |
| | II-restricted antigens: mutated CDC27 as a tumor antigen.” |
| | Science 284: 1351-4. |
| SART-1 | Genbank BC001058 [Homo sapiens squamous cell |
| squamous cell carcinoma | carcinoma antigen recognized by T cells, mRNA (cDNA |
| antigen recognized by T-cells | clone MGC: 2038 IMAGE: 3504745), complete cds] |
| | Hosokawa et al. (2005), “Cell cycle arrest and apoptosis |
| | induced by SART-1 gene transduction.” Anticancer Res. |
| | 25(3B): 1983-90. |
| EpCAM | Genbank BC014785 [Homo sapiens epithelial cell |
| epithelial cell adhesion molecule | adhesion molecule, mRNA (cDNA clone MGC: 9040 |
| | IMAGE: 3861826), complete cds] |
| | Munz et al. (2009), “The emerging role of EpCAM in |
| | cancer and stem cell signaling.” Cancer Res. 69(14): 5627-9. |
| CD20 | Genbank BC002807 [Homo sapiens membrane-spanning |
| also known as | 4-domains, subfamily A, member 1, mRNA (cDNA clone |
| membrane-spanning 4-domains, | MGC: 3969 IMAGE: 3634040), complete cds.] |
| subfamily A, member 1 | Tedder et al. (1988), “Isolation and structure of a cDNA |
| | encoding the B1 (CD20) cell-surface antigen of human B |
| | lymphocytes.” Proc. Natl. Acad. Sci. USA 85(1): 208-12. |
| CD23 | Genbank BC062591 [Homo sapiens Fc fragment of IgE, |
| also known as | low affinity II, receptor for (CD23), mRNA (cDNA clone |
| receptor for Fc fragment of IgE, | MGC: 74689 IMAGE: 5216918), complete cds] |
| low affinity II | Bund et al. (2007), “CD23 is recognized as tumor- |
| | associated antigen (TAA) in B-CLL by CD8+ autologous |
| | T lymphocytes.” Exp. Hematol. 35(6): 920-30. |
| CD33 | Genbank BC028152 [Homo sapiens CD33 molecule, |
| | mRNA (cDNA clone MGC: 40026 IMAGE: 5217182), |
| | complete cds] |
| | Peiper et al. (1988), “Molecular cloning, expression, and |
| | chromosomal localization of a human gene encoding the |
| | CD33 myeloid differentiation antigen.” Blood 72(1): 314-21. |
| EGFR | Genbank NM_005228 [Homo sapiens epidermal growth |
| epidermal growth factor | factor receptor (erythroblastic leukemia viral (v-erb-b) |
| receptor | oncogene homolog, avian) (EGFR), transcript variant 1, |
| | mRNA] |
| | Kordek et al. (1994), “Expression of a p53-protein, |
| | epidermal growth factor receptor (EGFR) and proliferating |
| | cell antigens in human gliomas.” Folia Neuropathol. 32(4): |
| | 227-8. |
| HER-2 | Genbank NM_001005862 [Homo sapiens v-erb-b2 |
| also known as | erythroblastic leukemia viral oncogene homolog 2, |
| v-erb-b2 erythroblastic leukemia | neuro/glioblastoma derived oncogene homolog (avian) |
| viral oncogene homolog 2, | (ERBB2), transcript variant 2, mRNA] |
| neuro/glioblastoma derived | Neubauer et al. (2008), “Changes in tumour biological |
| oncogene homolog (avian) | markers during primary systemic chemotherapy (PST).” |
| | Anticancer Res. 38(3B): 1797-804. |
| BTA-1 | [unable to locate a protein with this name] |
| breast tumor-associated antigen 1 | |
| BTA-2 | [unable to locate a protein with this name] |
| breast tumor-associated antigen 2 | |
| RCAS1 | Genbank BC022506 [Homo sapiens estrogen receptor |
| receptor-binding cancer antigen | binding site associated, antigen, 9, mRNA (cDNA clone |
| expressed on SiSo cells | MGC: 26497 IMAGE: 4815654), complete cds] |
| also known as | Giaginis et al. (2009), “Receptor-binding cancer antigen |
| estrogen receptor binding side | expressed on SiSo cells (RCAS1): a novel biomarker in the |
| associated antigen 9 | diagnosis and prognosis of human neoplasia.” Histol. |
| | Histopathol. 24(6): 761-76. |
| PLAC1 | Genbank BC022335 [Homo sapiens placenta-specific 1, |
| placenta-specific 1 | mRNA (cDNA clone MGC: 22788 IMAGE: 4769552), |
| | complete cds] |
| | Dong et al. (2008), “Plac1 is a tumor-specific antigen |
| | capable of eliciting spontaneous antibody responses in |
| | human cancer patients.” Int. J. Cancer 122(9): 2038-43. |
| syndecan | Genbank BC008765 [Homo sapiens syndecan 1, mRNA |
| | (cDNA clone MGC: 1622 IMAGE: 3347793), complete |
| | cds] |
| | Sun et al. (1997), “Large scale and clinical grade |
| | purification of syndecan-1 + malignant plasma cells.” J. |
| | Immunol. Methods 205(1): 73-9. |
| gp250 | Genbank BC137171 [Homo sapiens sortilin-related |
| also known as | receptor, L(DLR class) A repeats-containing, mRNA |
| sortilin-related receptor, L(DLR | (cDNA clone MGC: 168791 IMAGE: 9021168), complete |
| class) A repeats-containing | cds] |

Although various specific embodiments of the present invention have been described herein, it is to be understood that the invention is not limited to those precise embodiments and that various changes or modifications can be affected therein by one skilled in the art without departing from the scope and spirit of the invention.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention.

The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

The disclosures of all references and publications cited herein are expressly incorporated by reference in their entireties to the same extent as if each were incorporated by reference individually.

### REFERENCES

Aspberg, A., Miura, R., Bourdoulous, S., Shimonaka, M., Heinegard, D., Schachner, M., Ruoslahti, E., and Yamaguchi, Y. (1997). “The C-type lectin domains of lecticans, a family of aggregating chondroitin sulfate proteoglycans, bind tenascin-R by protein-protein interactions independent of carbohydrate moiety”. Proc. Natl. Acad. Sci. (USA) 94: 10116-10121
Bass, S., Greene, R., and Wells, J. A. (1990). “Hormone phage: an enrichment method for variant proteins with altered binding properties”. Proteins 8: 309-314
Benhar, I., Azriel, R., Nahary, L., Shaky, S., Berdichevsky, Y., Tamarkin, A., and Wels, W. (2000). “Highly efficient selection of phage antibodies mediated by display of antigen as Lpp-OmpA' fusions on live bacteria”. J. Mol. Biol. 301: 893-904
Berglund, L. and Petersen, T. E. (1992). “The gene structure of tetranectin, a plasminogen binding protein”. FEBS Letters 309: 15-19
Bertrand, J. A., Pignol, D., Bernard, J-P., Verdier, J-M., Dagorn, J-C., and Fontecilla-Camps, J. C. (1996). “Crystal structure of human lithostathine, the pancreatic inhibitor of stone formation”. EMBO J. 15: 2678-2684
Bettler, B., Texido, G., Raggini, S., Ruegg, D., and Hofstetter, H. (1992). “Immunoglobulin E-binding site in Fc epsilon receptor (Fc epsilon R11/CD23) identified by homolog-scanning mutagenesis”. J. Biol. Chem. 267: 185-191
Blanck, O., Iobst, S. T., Gabel, C., and Drickamer, K. (1996). “Introduction of selectin-like binding specificity into a homologous mannose-binding protein”. J. Biol. Chem. 271: 7289-7292
Boder, E. T. and Wittrup, K. D. (1997). “Yeast surface display for screening combinatorial polypeptide libraries”. Nature Biotech. 15: 553-557

Burrows L, Iobst S T, Drickamer K. (1997) “Selective binding of N-acetylglucosamine to the chicken hepatic lectin”. Bio-chem J. 324:673-680
Chiba, H., Sano, H., Saitoh, M., Sohma, H., Voelker, D. R., Akino, T., and Kuroki, Y. (1999). “Introduction of mannose binding protein-type phosphatidylinositol recognition into pulmonary surfactant protein A”. Biochemistry 38: 7321-7331
Christensen, J. H., Hansen, P. K., Lillelund, O., and Thogersen, H. C. (1991). “Sequence-specific binding of the N-terminal three-finger fragment of Xenopus transcription factor IIIA to the internal control region of a 5S RNA gene”. FEBS Letters 281: 181-184
Cyr, J. L. and Hudspeth, A. J. (2000). “A library of bacteriophage-displayed antibody fragments directed against proteins of the inner ear”. Proc. Natl. Acad. Sci. (USA) 97: 2276-2281
Drickamer, K. (1992). “Engineering galactose-binding activity into a C-type mannose-binding protein”. Nature 360: 183-186
Drickamer, K. and Taylor, M. E. (1993). “Biology of animal lectins”. Annu Rev. Cell Biol. 9: 237-264
Drickamer, K. (1999). “C-type lectin-like domains”. Curr. Opinion Struc. Biol. 9: 585-590
Dunn, I. S. (1996). “Phage display of proteins”. Curr. Opinion Biotech. 7: 547-553
Erbe, D. V., Lasky, L. A., and Presta, L. G. “Selectin variants”. U.S. Pat. No. 5,593,882
Ernst, W. J., Spenger, A., Toellner, L., Katinger, H., Grabherr, R. M. (2000). “Expanding baculovirus surface display. Modification of the native coat protein gp64 of Autographa californica NPV”. Eur. J. Biochem. 267: 4033-4039
Ewart, K. V., Li, Z., Yang, D. S.C., Fletcher, G. L., and Hew, C. L. (1998). “The ice-binding site of Atlantic herring antifreeze protein corresponds to the carbohydrate-binding site of C-type lectins”. Biochemistry 37: 4080-4085
Feinberg, H., Park-Snyder, S., Kolatkar, A. R., Heise, C. T., Taylor, M. E., and Weis, W. I. (2000). “Structure of a C-type carbohydrate recognition domain from the macrophage mannose receptor”. J. Biol. Chem. 275: 21539-21548
Fujii, I., Fukuyama, S., Iwabuchi, Y., and Tanimura, R. (1998). “Evolving catalytic antibodies in a phage-displayed combinatorial library”. Nature Biotech. 16: 463-467
Gates, C. M., Stemmer, W. P. C., Kaptein, R., and Schatz, P. J. (1996). “Affinity selective isolation of ligands from peptide libraries through display on a lac repressor “headpiece dimer”. J. Mol. Biol. 255: 373-386
Graversen, J. H., Lorentsen, R. H., Jacobsen, C., Moestrup, S. K., Sigurskjold, B. W., Thogersen, H. C., and Etzerodt, M. (1998). “The plasminogen binding site of the C-type lectin tetranectin is located in the carbohydrate recognition domain, and binding is sensitive to both calcium and lysine”. J. Biol. Chem. 273:29241-29246
Graversen, J. H., Jacobsen, C., Sigurskjold, B. W., Lorentsen, R. H., Moestrup, S. K., Thogersen, H. C., and Etzerodt, M. (2000). “Mutational Analysis of Affinity and Selectivity of Kringle-Tetranectin Interaction. Grafting novel kringle affinity onto the tetranectin lectin scaffold”. J. Biol. Chem. 275: 37390-37396
Griffiths, A. D. and Duncan, A. R. (1998). “Strategies for selection of antibodies by phage display”. Curr. Opinion Biotech. 9: 102-108
Holtet, T. L., Graversen, J. H., Clemmensen, I., Thogersen, H. C., and Etzerodt, M. (1997). “Tetranectin, a trimeric plasminogen-binding C-type lectin”. Prot. Sci. 6: 1511-1515
Honma, T., Kuroki, Y., Tzunezawa, W., Ogasawara, Y., Sohma, H., Voelker, D. R., and Akino, T. (1997). “The mannose-binding protein A region of glutamic acid185-alanine-221 can functionally replace the surfactant protein A region of glutamic acid195-phenylalanine-228 without loss of interaction with lipids and alveolar type II cells”. Biochemistry 36: 7176-7184
Huang, W., Zhang, Z., and Palzkill, T. (2000). “Design of potent beta-lactamase inhibitors by phage display of beta-lactamase inhibitory protein”. J. Biol. Chem. 275: 14964-14968
Hufton, S. E., van Neer, N., van den Beuken, T., Desmet, J., Sablon, E., and Hoogenboom, H. R. (2000). “Development and application of cytotoxic T lymphocyte-associated antigen 4 as a protein scaffold for the generation of novel binding ligands”. FEBS Letters 475: 225-231
Hakansson, K., Lim, N. K., Hoppe, H-J., and Reid, K. B. M. (1999). “Crystal structure of the trimeric alpha-helical coiled-coil and the three lectin domains of human lung surfactant protein D”. Structure Folding and Design 7: 255-264
Iobst, S. T., Wormald, M. R., Weis, W. I., Dwek, R. A., and Drickamer, K. (1994). “Binding of sugar ligands to Ca(2+)-dependent animal lectins. I. Analysis of mannose binding by site-directed mutagenesis and NMR”. J. Biol. Chem. 269: 15505-15511
Iobst, S. T. and Drickamer, K. (1994). “Binding of sugar ligands to Ca(2+)-dependent animal lectins. II. Generation of high-affinity galactose binding by site-directed mutagenesis”. J. Biol. Chem. 269: 15512-15519
Iobst, S. T. and Drickamer, K. (1996). “Selective sugar binding to the carbohydrate recognition domains of the rat hepatic and macrophage asialoglycoprotein receptors”. J. Biol. Chem. 271: 6686-6693
Jaquinod, M., Holtet, T. L., Etzerodt, M., Clemmensen, I., Thogersen, H. C., and Roepstorff, P. (1999). “Mass Spectrometric Characterisation of Post-Translational Modification and Genetic Variation in Human Tetranectin”. Biol. Chem. 380: 1307-1314
Kastrup, J. S., Nielsen, B. B., Rasmussen, H., Holtet, T. L., Graversen, J. H., Etzerodt, M., Thogersen, H. C., and Larsen, I. K. (1998). “Structure of the C-type lectin carbohydrate recognition domain of human tetranectin”. Acta. Cryst. D 54: 757-766
Kogan, T. P., Revelle, B. M., Tapp, S., Scott, D., and Beck, P. J. (1995). “A single amino acid residue can determine the ligand specificity of E-selectin”. J. Biol. Chem. 270: 14047-14055
Kolatkar, A. R., Leung, A. K., Isecke, R., Brossmer, R., Drickamer, K., and Weis, W. I. (1998). “Mechanism of N-acetylgalactosamine binding to a C-type animal lectin carbohydrate-recognition domain”. J. Biol. Chem. 273: 19502-19508
Lorentsen, R. H., Graversen, J. H., Caterer, N. R., Thogersen, H. C., and Etzerodt, M. (2000). “The heparin-binding site in tetranectin is located in the N-terminal region and binding does not involve the carbohydrate recognition domain”. Biochem. J. 347: 83-87
Marks, J. D., Hoogenboom, H. R., Griffiths, A. D., and Winter, G. (1992). “Molecular evolution of proteins on filamentous phage. Mimicking the strategy of the immune system”. J. Biol. Chem. 267: 16007-16010
Mann K, Weiss I M, Andre S, Gabius H J, Fritz M. (2000). “The amino-acid sequence of the abalone (Haliotis laevigata) nacre protein perlucin. Detection of a functional C-type lectin domain with galactose/mannose specificity”. Eur. J. Biochem. 267: 5257-5264
McCafferty, J., Jackson, R. H., and Chiswell, D. J. (1991). “Phage-enzymes: expression and affinity chromatography of functional alkaline phosphatase on the surface of bacterio-phage”. Prot. Eng. 4: 955-961
McCormack, F. X., Kuroki, Y., Stewart, J. J., Mason, R. J., and Voelker, D. R. (1994). “Surfactant protein A amino acids Glu195 and Arg197 are essential for receptor binding, phospholipid aggregation, regulation of secretion, and the facilitated uptake of phospholipid by type II cells”. J. Biol. Chem. 269: 29801-29807
McCormack, F. X., Festa, A. L., Andrews, R. P., Linke, M., and Walzer, P. D. (1997). “The carbohydrate recognition domain of surfactant protein A mediates binding to the major surface glycoprotein of Pneumocystis carinii”. Biochemistry 36: 8092-8099
Meier, M., Bider, M. D., Malashkevich, V. N., Spiess, M., and Burkhard, P. (2000). “Crystal structure of the carbohydrate recognition domain of the Hi subunit of the asialoglycoprotein receptor”. J. Mol. Biol. 300: 857-865
Mikawa, Y. G., Maruyama, I. N., and Brenner, S. (1996). “Surface display of proteins on bacteriophage lambda heads”. J. Mol. Biol. 262: 21-30
Mio H, Kagami N, Yokokawa S, Kawai H, Nakagawa S, Takeuchi K, Sekine S, Hiraoka A. (1998). “Isolation and characterization of a cDNA for human mouse, and rat full-length stem cell growth factor, a new member of C-type lectin superfamily”. Biochem. Biophys. Res. Commun. 249: 124-130
Mizuno, H., Fujimoto, Z., Koizumi, M., Kano, H., Atoda, H., and Morita, T. (1997). “Structure of coagulation factors IX/X-binding protein, a heterodimer of C-type lectin domains”. Nat. Struc. Biol. 4: 438-441 [0287] Ng, K. K., Park-Snyder, S., and Weis, W. I. (1998a). “Ca.sup.2+-dependent structural changes in C-type mannose-binding proteins”. Biochemistry 37: 17965-17976
Ng, K. K. and Weis, W. I. (1998b). “Coupling of prolyl peptide bond isomerization and Ca2+binding in a C-type mannose-binding protein”. Biochemistry 37: 17977-17989
Nielsen, B. B., Kastrup, J. S., Rasmussen, H., Holtet, T. L., Graversen, J. H., Etzerodt, M., Thogersen, H. C., and Larsen, I. K. (1997). “Crystal structure of tetranectin, a trimeric plasminogen-binding protein with an alpha-helical coiled coil”. FEBS Letters 412: 388-396
Nissim A., Hoogenboom, H. R., Tomlinson, I. M., Flynn, G., Midgley, C., Lane, D., and Winter, G. (1994). “Antibody fragments from a ‘single pot’ phage display library as immunochemical reagents”. EMBO J. 13: 692-698
Ogasawara, Y. and Voelker, D. R. (1995). “Altered carbohydrate recognition specificity engineered into surfactant protein D reveals different binding mechanisms for phosphatidylinositol and glucosylceramide”. J. Biol. Chem. 270: 14725-14732
Ohtani, K., Suzuki, Y., Eda, S., Takao, K., Kase, T., Yamazaki, H., Shimada, T., Keshi, H., Sakai, Y., Fukuoh, A., Sakamoto, T., and Wakamiya, N. (1999). “Molecular cloning of a novel human collectin from liver (CL-L1)”. J. Biol. Chem. 274: 13681-13689
Pattanajitvilai, S., Kuroki, Y., Tsunezawa, W., McCormack, F. X., and Voelker, D. R. (1998). “Mutational analysis of Arg197 of rat surfactant protein A. His197 creates specific lipid uptake defects”. J. Biol. Chem. 273: 5702-5707
Poget, S. F., Legge, G. B., Proctor, M. R., Butler, P. J., Bycroft, M., and Williams, R. L. (1999). “The structure of a tunicate C-type lectin from Polyandrocarpa misakiensis complexed with D-galactose”. J. Mol. Biol. 290: 867-879
Revelle, B. M., Scott, D., Kogan, T. P., Zheng, J., and Beck, P. J. (1996). “Structure-function analysis of P-selectinsialyl LewisX binding interactions. Mutagenic alteration of ligand binding specificity”. J. Biol. Chem. 271: 4289-4297
Sano, H., Kuroki, Y., Honma, T., Ogasawara, Y., Sohma, H., Voelker, D. R., and Akino, T. (1998). “Analysis of chimeric proteins identifies the regions in the carbohydrate recognition domains of rat lung collections that are essential for interactions with phospholipids, glycolipids, and alveolar type II cells”. J. Biol. Chem. 273: 4783-4789
Schaffitzel, C., Hanes, J., Jermutus, L., and Plucktun, A. (1999). “Ribosome display: an in vitro method for selection and evolution of antibodies from libraries”. J. Immunol. Methods 231: 119-135
Sheriff, S., Chang, C. Y., and Ezekowitz, R. A. (1994). “Human mannose-binding protein carbohydrate recognition domain trimerizes through a triple alpha-helical coiled-coil”. Nat. Struc. Biol. 1: 789-794
Sorensen, C. B., Berglund, L., and Petersen, T. E. (1995). “Cloning of a cDNA encoding murine tetranectin”. Gene 152: 243-245
Torgersen, D., Mullin, N. P., and Drickamer, K. (1998). “Mechanism of ligand binding to E- and P-selectin analyzed using selectin/mannose-binding protein chimeras”. J. Biol. Chem. 273: 6254-6261
Tormo, J., Natarajan, K., Margulies, D. H., and Mariuzza, R. A. (1999). “Crystal structure of a lectin-like natural killer cell receptor bound to its MHC class I ligand”. Nature 402: 623-631
Tsunezawa, W., Sano, H., Sohma, H., McCormack, F. X., Voelker, D. R., and Kuroki, Y. (1998). “Site-directed mutagenesis of surfactant protein A reveals dissociation of lipid aggregation and lipid uptake by alveolar type II cells”. Biochim. Biophys. Acta 1387: 433-446
Weis, W. I., Kahn, R., Fourme, R., Drickamer, K., and Hendrickson, W. A. (1991). “Structure of the calcium-dependent lectin domain from a rat mannose-binding protein determined by MAD phasing”. Science 254: 1608-1615
Weis, W. I., and Drickamer, K. (1996). “Structural basis of lectin-carbohydrate recognition”. Annu Rev. Biochem. 65: 441-473
Whitehorn, E. A., Tate, E., Yanofsky, S. D., Kochersperger, L., Davis A., Mortensen, R. B., Yonkovic, S., Bell, K., Dower, W. J., and Barrett, R. W. (1995). “A generic method for expression and use of “tagged” soluble versions of cell surface receptors”. Bio/Technology 13: 1215-1219
Wragg, S, and Drickamer, K. (1999). “Identification of amino acid residues that determine pH dependence of ligand binding to the asialoglycoprotein receptor during endocytosis”. J. Biol. Chem. 274: 35400-35406
Zhang, H., Robison, B., Thorgaard, G. H., and Ristow, S. S. (2000). “Cloning, mapping and genomic organization of a fish C-type lectin gene from homozygous clones of rainbow trout (Oncorhynchos Mykiss)”. Biochim. et Biophys. Acta 1494: 14-22
Agnew, Chem. Intl. Ed. Engl., 33: 183-186 (1994)
Ashkenazi, et al. JClin Invest.; 104(2):155-62 (July 1999).
Chemotherapy Service Ed., M. C. Perry, Williams & Wilkins, Baltimore, Md. (1992)
Ausubel et al., Current Protocols in Molecular Biology (eds., Green Publishers Inc. and Wiley and Sons 1994
Degli-Esposti et al., Immunity, 7(6):813-820 (December 1997)
Degli-Esposti et al., J. Exp. Med., 186(7):1165-1170 (Oct. 6, 1997)
Janeway, Nature, 341(6242): 482-3 (Oct. 12, 1989)
Jin et al, Cancer Res., 15; 64(14):4900-5 (July 2004).
Langer et al., J. Biomed. Mater. Res., 15: 167-277 (1981)
Langer, Chem. Tech., 12: 98-105 (1982)
Marsters et al., Curr. Biol., 7:1003-1006 (1997)
McFarlane et al., J. Biol. Chem., 272:25417-25420 (1997)
Mongkolsapaya et al., J. Immunol., 160:3-6 (1998)
Mordenti et al., Pharmaceut. Res., 8:1351 (1991)
Neame, et al., Protein Sci., 1(1):161-8 (1992)
Neame, P. J. and Boynton, R. E., Protein Soc. Symposium, (Meeting date 1995; 9th Meeting: Tech. Prot. Chem. VII). Proceedings pp. 401-407 (Ed., Marshak, D. R.; Publisher: Academic, San Diego, Calif.) (1996).
Offner et al., Science, 251: 430-432 (1991)
Pan et al., FEBS Letters, 424:41-45 (1998)
Pan et al., Science, 276:111-113 (1997)
Pan et al., Science, 277:815-818 (1997)
Remington's Pharmaceutical Sciences, 16th edition, Osol, A. ed. (1980)
S. G. Hymowitz, et. al., Mol Cell. 1999 October; 4(4):563-71)
Sambrook, et al. Molecular Cloning: A Laboratory Manual. Cold Spring Harbor Laboratory Press, Cold Spring Harbor, N.Y. (1989)
Schneider et al., FEBS Letters, 416:329-334 (1997)
Screaton et al., Curr. Biol., 7:693-696 (1997)
Sheridan et al., Science, 277:818-821 (1997)
Sidman et al., Biopolymers, 22: 547-556 (1983)
Cha et. al., J Biol. Chem., 275(40):31171-7 (Oct. 6, 2000).
Murakami et al., The Molecular Basis of Cancer, Mendelsohn and Israel, eds., Chapter 1, entitled “Cell cycle regulation, oncogenes, and antineoplastic drugs” by (WB Saunders: Philadelphia, pg. 13 (1995).
Walczak et al., EMBO J., 16:5386-5387 (1997)
Wu et al., Nature Genetics, 17:141-143 (1997)

What is claimed is:

1. A polypeptide comprising a trimerizing domain and at least one polypeptide sequence that binds to human IL-23R without activating IL-23 heterodimeric receptor.

2. The polypeptide of claim 1, wherein the polypeptide does not bind to at least one of human IL-12Rβ1 or human IL-12Rβ2.

3. The polypeptide of claim 1, wherein the polypeptide competes with native human IL-23 for binding to human IL-23R.

4. The polypeptide of claim 1 wherein the trimerizing domain comprises a polypeptide of a human tetranectin trimerizing domain (SEQ ID NO: 99) having up to five amino acid substitutions at positions 26, 30, 33, 36, 37, 40, 31, 42, 45, 46, 47, 48, 49, 50 and 51 and wherein three trimerizing domains form a trimeric complex.

5. The polypeptide of claim 1 wherein the trimerizing domain comprises a trimerizing polypeptide selected from the group consisting of hTRAF3 [SEQ ID NO: 191], hMBP [SEQ ID NO: 192], hSPC300 [SEQ ID NO: 193], hNEMO [SEQ ID NO: 194], hcubilin [SEQ ID NO: 195], hThrombospondins [SEQ ID NO: 196], and neck region of human SP-D, [SEQ ID NO: 197], neck region of bovine SP-D [SEQ ID NO: 198], neck region of rat SP-D [SEQ ID NO: 199], neck region of bovine conglutinin: [SEQ ID NO: 200]; neck region of bovine collectin: [SEQ ID NO: 201]; and neck region of human SP-D: [SEQ ID NO: 202].

6. The polypeptide of claim 1 wherein the human IL-23R comprises SEQ ID NO: 5.

7. The polypeptide of claim 1, wherein the at least one polypeptide that binds IL-23R is linked to one of the N-terminus and the C-terminus of the trimerizing domain, and further comprising a modulator of inflammation positioned at the other of the N-terminus and the C-terminus.

8. The polypeptide of claim 1, wherein the at least one polypeptide that binds to IL-23R comprises a C-Type Lectin Like Domain (CLTD) and wherein one of loops 1, 2, 3 or 4 of loop segment A or loop segment B of the CTLD comprises a polypeptide sequence that binds IL-23.

9. The polypeptide of claim 7, wherein the polypeptide sequence of the CTLD is selected from the group consisting of SEQ ID NO: 133, 134, 135, 167, 137, 138, 139, 140, and 141.

10. The polypeptide of claim 1, wherein the polypeptide that binds IL-23 is linked to one of the N-terminus and the C-terminus of the trimerizing domain, and further comprising a modulator of inflammation positioned at the other of the N-terminus and the C-terminus.

11. The polypeptide of claim 1 having a polypeptide that binds IL-23 linked to each of the N-terminus and the C-terminus, wherein the polypeptide at the N-terminus is the same or different than the polypeptide at the C-terminus.

12. The polypeptide of claim 1 wherein the polypeptide is a fusion protein.

13. The polypeptide of claim 1 wherein the polypeptide that binds IL-23R is positioned at one of the N-terminus and the C-terminus of the trimerizing domain, and further comprising a polypeptide sequence that binds a tumor-associated antigen (TAA) or tumor-specific antigen (TSA) at the other of the N-terminus and the C-terminus.

14. The polypeptide of claim 1 further comprising a therapeutic agent covalently attached to the polypeptide.

15. A trimeric complex comprising three polypeptides of claim 1.

16. The trimeric complex of claim 15 wherein the trimerizing domain is a tetranectin trimerizing structural element.

17. A method of preventing activation of IL-23R by IL-23 in cells that express IL-23R, the method comprising contacting the cell with the trimeric complex of claim 15.

18. A pharmaceutical composition comprising the trimeric complex of claim 16 and at least one pharmaceutically acceptable excipient.

19. A method for treating an immune disorder in a subject comprising administering to the animal the pharmaceutical composition of claim 18.

20. The method of claim 19, further comprising administering to the subject, either simultaneously or sequentially, a modulator of inflammation.

21. A method for treating cancer in an animal comprising administering to a subject in need therefore the pharmaceutical composition of claim 18.

22. The method of claim 21, further comprising administering to the animal, either simultaneously or sequentially, at least one of chemotherapeutic agent or a cytotoxic agent.

23. A method for preparing the polypeptide of claim 1 comprising:
a) selecting a first polypeptide that binds to IL-23R; and
b) fusing the first polypeptide with one of the N-terminus or the C-terminus of a multimerizing domain.

24. The method of claim 23 further comprising:
a) selecting a second polypeptide sequence that is a modulator of inflammation; and
b) fusing the second polypeptide with the other of the N-terminus or the C-terminus of the multimerizing domain.

25. The method of claim 21 wherein step (a) the polypeptide is selected so that it does not bind to at least one of IL-12Rβ1 or IL-12Rβ2.

26. A method for preparing a polypeptide complex that prevents activation of a IL-23R in a cell expressing IL-23R comprising trimerizing three polypeptides prepared according to claim 23.

27. A method for preparing a polypeptide that mediates an immune related disorder comprising:
a) creating a library of polypeptides comprising a CTLD comprising at least one randomized loop region;
b) selecting a first polypeptide from the library that binds IL-23R but does not bind to at least one of IL-12Rβ1 or IL-12Rβ2.

28. The method of claim 27, further comprising: (c) attaching the selected polypeptide to the N-terminus or the C-terminus of a multimerizing domain.

29. A polypeptide that competes with native human IL-23 for binding to native IL-23R, wherein the polypeptide does not activate human IL-23R and does not bind to at least one of IL-12Rβ1 or IL-12Rβ2.

30. The polypeptide of claim 30 wherein, the polypeptide is a CTLD that has been modified in one of loops 1, 2, 3 or 4 of loop segment A or in loop segment B for binding to IL-23R.

31. The polypeptide of claim 30 comprising a polypeptide selected from the group consisting of SEQ ID NO: 133, 134, 135, 167, 137, 138, 139, 140, and 141.

32. An isolated polynucleotide encoding a polypeptide comprising the polypeptide of claim 1.

33. A vector comprising the polynucleotide of claim 32.

34. A host cell comprising the vector of claim 34.
